(19) United States
(12) Patent Application Publication
(10) Pub. No.: US 2021/0010065 A1
(43) Pub. Date: Jan. 14, 2021

(54) METHODS AND REAGENTS FOR ENRICHMENT OF NUCLEIC ACID MATERIAL FOR SEQUENCING APPLICATIONS AND OTHER NUCLEIC ACID MATERIAL INTERROGATIONS

(71) Applicant: TwinStrand Biosciences, Inc., Seattle, WA (US)

(72) Inventors: Jesse J. SALK, Seattle, WA (US); Lindsey Nicole WILLIAMS, Seattle, WA (US); Tan LI, Seattle, WA (US)

(21) Appl. No.: 16/980,706

(22) PCT Filed: Mar. 15, 2019
(86) PCT No.: PCT/US2019/022640

§ 371 Date: Sep. 14, 2020

Related U.S. Application Data
(60) Provisional application No. 62/643,738, filed on Mar. 15, 2018.

(51) Int. Cl. C12Q 1/6806; C12N 9/22; C12Q 1/686; C12N 9/12; C12Q 1/6818

(52) U.S. Cl. CPC C12Q 1/6806; C12N 9/22; C12Q 1/686; C12Q 2531/113; C12Q 1/6818; C12N 2310/531; C12N 2310/20; C12N 9/1276

(57) ABSTRACT

The present technology relates generally to methods and compositions for targeted nucleic acid sequence enrichment, as well as uses of such enrichment for error-corrected nucleic acid sequencing applications and other nucleic acid sequence interrogations. In some embodiments, provided methods provide non-amplification based targeted enrichment strategies compatible with the use of molecular barcodes for error correction. Other embodiments provide methods for non-amplification based targeted enrichment strategies compatible with direct digital sequencing (DDS) and other sequencing strategies (e.g., single molecule sequencing modalities and interrogations) that do not use molecular barcoding.

### CROSS-REFERENCE TO RELATED APPLICATIONS

This application claims priority to and the benefit of U.S. Provisional Patent Application No. 62/643,738, filed Mar. 15, 2018, the disclosure of which are hereby incorporated by reference in their entirety.

### BACKGROUND

A variety of approaches at the level of protocol development, chemistry/biochemistry and data processing have been developed to mitigate the impact of PCR-based errors in massively parallel sequencing (MPS, also sometimes known as next generation DNA sequencing, NGS) applications. In addition, techniques whereby PCR duplicates arising from individual DNA fragments can be resolved on the basis of unique random shear points or via exogenous tagging (i.e. using molecular bar codes, also known as molecular tags, unique molecular identifiers [UMIs] and single molecule identifiers [SMIs]), before or during amplification are in common use. This approach has been used to improve counting accuracy of DNA and RNA templates. Because all amplicons derived from a single starting molecule can be explicitly identified, any variation in the sequence of identically tagged sequencing reads can be used to correct base errors arising during PCR or sequencing. For instance, Kinde, et al. (Proc Natl Acad Sci USA 108, 9530-9535, 2011) introduced SafeSeqS, which uses single-stranded molecular barcoding to reduce the error rate of sequencing by grouping PCR copies sharing the barcode sequencing and forming a consensus. However, the incorporation of a single-stranded molecular barcode cannot fully eliminate PCR artifacts arising in the first round of amplification that get carried onto derivative copies as a “jackpot” event.

Methods for higher accuracy genotyping of single nucleotide polymorphism (SNP) loci, short tandem repeat (STR) loci, and many other forms of mutations and genetic variants are desirable in a variety of applications in medicine, forensics, genotoxicology, and other science industry applications. A challenge, however, is how to most efficiently generate sequence information from as many relevant copies of genetic material being sequenced as possible with the highest confidence but at a reasonable cost. Various consensus sequencing methods (both molecular barcode-based and not) have been used successfully for error correction to help better identify variants in mixtures (see J. Salk et al, Enhancing the accuracy of next-generation sequencing for detecting rare and subclonal mutations, Nature Reviews Genetics, 2018, for detailed discussion), but with various tradeoffs in performance. We have previously described Duplex Sequencing, an ultra-high accuracy sequencing method that relies on genotyping and comparing the independent strand sequenced of double stranded nucleic acid molecules for the purpose of error correction. Aspects of the technology articulated herein describes methods for improving cost efficiency, recovery efficiency, and other performance metrics as well as overall process speed for Duplex Sequencing and other sequencing applications for achieving high accuracy sequencing reads.

### SUMMARY

The present technology relates generally to methods for targeted nucleic acid sequence enrichment and uses of such enrichment for error-corrected nucleic acid sequencing applications and other nucleic acid material interrogations. In some embodiments, highly accurate, error-corrected and massively parallel sequencing of nucleic acid material is possible using target nucleic acid material that has been enriched from a sample. In some aspects, the target enriched nucleic acid material is double-stranded and one or more methods of uniquely labeling strands of double-stranded nucleic acid complexes can be used in such a way that each strand can be informatically related to its complementary strand, but also distinguished from it following sequencing of each strand or an amplified product derived therefrom, and this information can be further used for the purpose of error correction of the determined sequence. Some aspects of the present technology provide methods and compositions for improving the cost, conversion of molecules sequenced and the time efficiency of generating labeled molecules for targeted ultra-high accuracy sequencing. In some embodiments, provided methods and compositions allow for the accurate analysis of very small amounts of nucleic acid material (e.g., from a small clinical sample or DNA floating freely in blood or a sample taken from a crime scene). In some embodiments, provided methods and compositions allow for the detection of mutations in a sample of a nucleic acid material that are present at a frequency less than one in one hundred cells or molecules (e.g., less than one in one thousand cells or molecules, less than one in ten thousand cells or molecules, less than one in one hundred thousand cells or molecules).

Aspects of the present technology are directed methods for enriching target nucleic acid material that include, providing a nucleic acid material, and cutting the nucleic acid material with one or more targeted endonucleases so that a target region of predetermined length is separated from the rest of the nucleic acid material. The methods can further include enzymatically destroying non-targeted nucleic acid material, releasing the target region of predetermined length from the targeted endonuclease; and analyzing the cut target region.

Additional aspects of the present technology are directed to methods for enriching target nucleic acid material that include providing a nucleic acid material, cutting the nucleic acid material with one or more targeted endonucleases so that a target region of predetermined length is separated from the rest of the nucleic acid material, wherein at least one targeted endonuclease comprises a capture label; capturing the target region of predetermined length with an extraction moiety configured to bind the capture label; releasing the target region of predetermined length from the targeted endonuclease; and analyzing the cut target region.

Further aspects of the present technology are directed methods for enriching target nucleic acid material, comprising providing a nucleic acid material; binding a catalytically inactive CRISPR-associated (Cas) enzymes to a target region of the nucleic acid material; enzymatically treating the nucleic acid material with one or more nucleic acid digesting enzymes such that non-targeted nucleic acid material is destroyed and the target region is protected from the digesting enzymes by the bound catalytically inactive Cas enzyme; releasing the target region from the catalytically inactive Cas enzyme; and analyzing the target region.

Another aspect of the present technology is directed to methods for enriching target nucleic acid material, comprising providing a nucleic acid material; providing a pair of catalytically active targeted endonucleases and at least one catalytically inactive targeted endonuclease comprising a capture label, wherein the catalytically inactive targeted endonuclease is directed to bind the target region of the nucleic acid material, and wherein the pair of catalytically active targeted endonucleases are directed to bind the target region on either side of the catalytically inactive targeted endonuclease; cutting the nucleic acid material with the pair of catalytically active targeted endonucleases so that the target region is separated from the rest of the nucleic acid material; capturing the target region with an extraction moiety configured to bind the capture label; releasing the target region from the targeted endonucleases; and analyzing the cut target region.

Further aspects include methods for enriching target nucleic acid material from a sample comprising a plurality of nucleic acid fragments, comprising providing one or more catalytically inactive CRISPR-associated (Cas) enzymes having a capture label to the sample comprising target nucleic acid fragments and non-target nucleic acid fragments, wherein the one or more catalytically inactive Cas enzymes are configured to bind the target nucleic acid fragments; providing a surface comprising an extraction moiety configured to bind the capture label; and separating the target nucleic acid fragments from the non-target nucleic acid fragments by capturing the target nucleic acid fragments via binding the capture label by the extraction moiety.

Various embodiments provide methods for enriching target double-stranded nucleic acid material, comprising providing a nucleic acid material; cutting the nucleic acid material with one or more targeted endonucleases to generate a double-stranded target nucleic acid fragment comprising 5′ sticky end having a 5′ predetermined nucleotide sequence and/or a 3′ sticky end having a 3′ predetermined nucleotide sequence; and separating the double-stranded target nucleic acid molecule from the rest of the nucleic acid material via at least one of the 5′ sticky end and the 3′ sticky end.

Additional embodiments provide kits for enriching target nucleic acid material, comprising nucleic acid library, comprising nucleic acid material, and a plurality of catalytically inactive Cas enzymes, wherein the Cas enzymes comprise a tag having a sequence code, and wherein the plurality of Cas enzymes are bound to a plurality of site-specific target regions along the nucleic acid material. The kits further comprise a plurality of probes, wherein each probe comprises an oligonucleotide sequence comprising a complement to a corresponding sequence code, and a capture label. Kits may also include a look-up table cataloguing the relationship between the site-specific target regions, the sequence code associated with the site-specific target region, and the probe comprising the complement to a corresponding sequence code.

In some embodiments, an error-corrected sequence read is used to identify or characterize a cancer, a cancer risk, a cancer mutation, a cancer metabolic state, a mutator phenotype, a carcinogen exposure, a toxin exposure, a chronic inflammation exposure, an age, a neurodegenerative disease, a pathogen, a drug resistant variant, a fetal molecule, a forensically relevant molecule, an immunologically relevant molecule, a mutated T-cell receptor, a mutated B-cell receptor, a mutated immunoglobulin locus, a kategis site in a genome, a hypermutable site in a genome, a low frequency variant, a subclonal variant, a minority population of molecules, a source of contamination, a nucleic acid synthesis error, an enzymatic modification error, a chemical modification error, a gene editing error, a gene therapy error, a piece of nucleic acid information storage, a microbial quasispecies, a viral quasispecies, an organ transplant, an organ transplant rejection, a cancer relapse, residual cancer after treatment, a preneoplastic state, a dysplastic state, a microchimerism state, a stem cell transplant state, a cellular therapy state, a nucleic acid label affixed to another molecule, or a combination thereof in an organism or subject from which the double-stranded target nucleic acid molecule is derived. In some embodiments, an error-corrected sequence read is used to identify a carcinogenic compound or exposure. In some embodiments, an error-corrected sequence read is used to identify a mutagenic compound or exposure. In some embodiments, a nucleic acid material is derived from a forensics sample, and the error-corrected sequence read is used in a forensic analysis.

In some embodiments, a single molecule identifier sequence comprises an endogenous shear point or an endogenous sequence that can be positionally related to the shear point. In some embodiments, a single molecule identifier sequence is at least of one of a degenerate or semi-degenerate barcode sequence, one or more nucleic acid fragment ends of the nucleic acid material, or a combination thereof that uniquely labels the double-stranded nucleic acid molecule. In some embodiments, the adapter and/or an adapter sequence comprises at least one nucleotide position that is at least partially non-complimentary or comprises at least one non-standard base. In some embodiments, an adapter comprises a single “U-shaped” oligonucleotide sequence formed by about 5 or more self-complementary nucleotides.

In accordance with various embodiments, any of a variety of nucleic acid material may be used. In some embodiments, nucleic acid material may comprise at least one modification to a polynucleotide within the canonical sugar-phosphate backbone. In some embodiments, nucleic acid material may comprise at least one modification within any base in the nucleic acid material. For example, by way of non-limiting example, in some embodiments, the nucleic acid material is or comprises at least one of double-stranded DNA, double-stranded RNA, peptide nucleic acids (PNAs), locked nucleic acids (LNAs).

In some embodiments, provided methods further comprise ligating adapter molecules to a double stranded nucleic acid molecule. In some embodiments a ligating step includes ligating a double-stranded nucleic acid material to at least one double-stranded degenerate barcode sequence to form a double-stranded nucleic acid molecule barcode complex, wherein the double-stranded degenerate barcode sequence comprises the single molecule identifier sequence in each strand. In some embodiments, the double stranded nucleic acid molecule is a double stranded DNA molecule or a double stranded RNA molecule. In some embodiments, the double stranded nucleic acid molecule comprises at least one modified nucleotide or non-nucleotide molecule.

In some embodiments, ligating comprises activity of at least one ligase. In some embodiments, the at least one ligase is selected from a DNA ligase and a RNA ligase. In some embodiments, ligating comprises ligase activity at a ligation domain associated with an adapter molecule. In some embodiments, ligating comprises ligase activity at a ligation domain associated with an adapter molecule and a ligatable end of a nucleic acid molecule. In some embodiments, the ligation domain and the ligatable end of a double-stranded nucleic acid molecule are compatible (e.g., have single-stranded regions that are complementary to each other). In some embodiments, the ligation domain is a nucleotide sequence from or in association with one or more degenerate or semi-degenerate nucleotides. In some embodiments, the ligation domain is a nucleotide sequence from one or more non-degenerate nucleotides. In some embodiments, the ligation domain contains one or more modified nucleotides. In some embodiments, the ligation domain and/or the ligatable end comprises a T-overhang, an A-overhang, a CG-overhang, a blunt end, a recombination sequence, an endonuclease cut site overhang, a restriction digest overhang, or another ligateable region. In some embodiments, at least one strand of the ligation domain is phosphorylated. In some embodiments, the ligation domain comprises an endonuclease cleavage sequence or a portion thereof.

In some embodiments, the endonuclease cleavage sequence is cleaved by an endonuclease (e.g., a tunable endonuclease, a restriction endonuclease) to yield a blunt end, or overhang with a ligateable region. In some embodiments, the ligatable end of a double-stranded nucleic acid molecule comprises an endonuclease cleavage sequence or a portion thereof. In some embodiments, an endonuclease (e.g., a programmable/targeted endonuclease, restriction endonuclease) yields an overhang comprising a “sticky end” or single-stranded overhang region with known nucleotide length (e.g., 1, 2, 3, 4, 5, 6, 7, 8, 9, 10, 11, 12, 13, 14, 15, 16, 17, 18, 19, 20 or more nucleotides) and sequence.

In some embodiments, an identifier sequence is or comprises a single molecule identifier (SMI) sequence. In some embodiments, a SMI sequence is an endogenous SMI sequence. In some embodiments, the endogenous SMI sequence is related to shear point. In some embodiments, the SMI sequence comprises at least one degenerate or semi-degenerate nucleic acid. In some embodiments, the SMI sequence is non-degenerate. In some embodiments, the SMI sequence is a nucleotide sequence of one or more degenerate or semi-degenerate nucleotides. In some embodiments, the SMI sequence is a nucleotide sequence of one or more non-degenerate nucleotides. In some embodiments, the SMI sequence comprises at least one modified nucleotide or non-nucleotide molecule. In some embodiments, the SMI sequence comprises a primer binding domain

In some embodiments, a modified nucleotide or non-nucleotide molecule is selected from 2-Aminopurine, 2,6-Diaminopurine (2-Amino-dA), 5-Bromo dU, deoxyUridine, Inverted dT, Inverted Dideoxy-T, Dideoxy-C, 5-Methyl dC, deoxyInosine, Super T®, Super G®, Locked Nucleic Acids, 5-Nitroindole, 2′-O-Methyl RNA Bases, Hydroxymethyl dC, Iso-dG, Iso-dC, Fluoro C, Fluoro U, Fluoro A, Fluoro G, 2-MethoxyEthoxy A, 2-MethoxyEthoxy MeC, 2-MethoxyEthoxy G, 2-MethoxyEthoxy T, 8-oxo-A, 8-oxoG, 5-hydroxymethyl-2′-deoxycytidine, 5′-methylisocytosine, tetrahydrofuran, iso-cytosine, iso-guanosine, uracil, methylated nucleotide, RNA nucleotide, ribose nucleotide, 8-oxo-G, BrdU, Loto dU, Furan, fluorescent dye, azide nucleotide, abasic nucleotide, 5-nitroindole nucleotide, and digoxenin nucleotide.

In some embodiments, a cut site is or comprises a restriction endonuclease recognition sequence. In some embodiments, a cut site is or comprises a user-directed recognition sequence for a targeted endonuclease (e.g., a CRISPR or CRISPR-like endonuclease) or other tunable endonuclease. In some embodiments, cutting nucleic acid material may comprise at least one of enzymatic digestion, enzymatic cleavage, enzymatic cleavage of one strand, enzymatic cleavage of both strands, incorporation of a modified nucleic acid followed by enzymatic treatment that leads to cleavage or one or both strands, incorporation of a replication blocking nucleotide, incorporation of a chain terminator, incorporation of a photocleavable linker, incorporation of a uracil, incorporation of a ribose base, incorporation of an 8-oxo-guanine adduct, use of a restriction endonuclease, use of a ribonucleoprotein endonuclease (e.g., a Cas-enzyme, such as Cas9 or CPF1), or other programmable endonuclease (e.g., a homing endonuclease, a zinc-fingered nuclease, a TALEN, a meganuclease (e.g., megaTAL nuclease), an argonaute nuclease, etc.), and any combination thereof.

In some embodiments, a capture label is or comprises at least one of Acrydite, azide, azide (NHS ester), digoxigenin (NHS ester), I-Linker, Amino modifier C6, Amino modifier C12, Amino modifier C6 dT, Unilink amino modifier, hexynyl, 5-octadiynyl dU, biotin, biotin (azide), biotin dT, biotin TEG, dual biotin, PC biotin, desthiobiotin TEG, thiol modifier C3, dithiol, thiol modifier C6 S—S, and succinyl groups.

In some embodiments, an extraction moiety is or comprises at least one of amino silane, epoxy silane, isothiocyanate, aminophenyl silane, aminpropyl silane, mercapto silane, aldehyde, epoxide, phosphonate, streptavidin, avidin, a hapten recognizing an antibody, a particular nucleic acid sequence, magnetically attractable particles (Dynabeads), and photolabile resins.

In some embodiments, provided methods further comprise amplifying nucleic acid material through use of a primer specific an adapter sequence and/or through use of a primer specific to a non-adapter portion of a nucleic acid product. It is contemplated that any of a variety of methods for amplifying nucleic acid material may be used in accordance with various embodiments. For example, in some embodiments, at least one amplifying step comprises a polymerase chain reaction (PCR), rolling circle amplification (RCA), multiple displacement amplification (MDA), isothermal amplification, polony amplification within an emulsion, bridge amplification on a surface, the surface of a bead or within a hydrogel, and any combination thereof. In some embodiments, amplifying a nucleic acid material includes use of single-stranded oligonucleotides at least partially complementary to regions of a first adapter sequence and a second adapter sequence (e.g., at least partially complementary to an adapter sequence on the 5′ and/or 3′ ends of each strand of the nucleic acid material). In some embodiments, amplifying a nucleic acid material includes use of a single-stranded oligonucleotide at least partially complementary to a region of a genomic sequence of interest and a single-stranded oligonucleotide at least partially complementary to a region of the adapter sequence.

In some embodiments, amplifying the nucleic acid material includes generating a plurality of amplicons derived from the first strand and a plurality of amplicons derived from the second strand.

In some embodiments, provided methods further comprise the steps of cutting the nucleic acid material with one or more targeted endonucleases such that a target nucleic acid fragment of a substantially known length is formed, and isolating the target nucleic acid fragment based on the substantially known length. In some embodiments, provided methods further comprise ligating an adapter (e.g., an adapter sequence) to a target nucleic acid (e.g., a target nucleic acid fragment) of substantially known length (e.g., following a size-enrichment step).

In some embodiments, a nucleic acid material may be or comprise one or more target nucleic acid fragments. In some embodiments, one or more target nucleic acid fragments each comprise a genomic sequence of interest from one or more locations in a genome. In some embodiments, one or more target nucleic acid fragments comprise a targeted sequence from a substantially known region within a nucleic acid material. In some embodiments, isolating a target nucleic acid fragment based on a substantially known length includes enriching for the target nucleic acid fragment by gel electrophoresis, gel purification, liquid chromatography, size exclusion purification, filtration or SPRI bead purification.

In some embodiments, provided methods further comprise the steps of cutting the double-stranded nucleic acid material with one or more targeted endonucleases such that a double-stranded target nucleic acid fragment comprising one or both ends having a substantially known length and/or sequence of single-strand overhang is formed. In some embodiments, provided methods further comprises the steps of isolating the double-stranded target nucleic acid fragment based on the substantially known length and/or sequence of single-strand overhang. In some embodiments, provided methods further comprise ligating an adapter (e.g., an adapter sequence) to a double-stranded target nucleic acid (e.g., a target nucleic acid fragment) having a substantially known length and/or sequence of single-stranded overhang. In some embodiments, a double-stranded target nucleic acid can have a ligatable end substantially uniquely compatible (e.g., complimentary) with a ligation domain of a ligation-selected adapter molecule such that one or more target nucleic acid fragments comprising a targeted sequence from a substantially known region within a nucleic acid material can be selectively enriched by way of amplification with primers specific to an adapter sequence that is associated with the ligation-selected adapter(s).

In accordance with various embodiments, some provided methods may be useful in sequencing any of a variety of suboptimal (e.g., damaged or degraded) samples of nucleic acid material. For example, in some embodiments at least some of the nucleic acid material is damaged. In some embodiments, the damage is or comprises at least one of oxidation, alkylation, deamination, methylation, hydrolysis, hydroxylation, nicking, intra-strand crosslinks, inter-strand cross links, blunt end strand breakage, staggered end double strand breakage, phosphorylation, dephosphorylation, sumoylation, glycosylation, deglycosylation, putrescinylation, carboxylation, halogenation, formylation, single-stranded gaps, damage from heat, damage from desiccation, damage from UV exposure, damage from gamma radiation damage from X-radiation, damage from ionizing radiation, damage from non-ionizing radiation, damage from heavy particle radiation, damage from nuclear decay, damage from beta-radiation, damage from alpha radiation, damage from neutron radiation, damage from proton radiation, damage from cosmic radiation, damage from high pH, damage from low pH, damage from reactive oxidative species, damage from free radicals, damage from peroxide, damage from hypochlorite, damage from tissue fixation such formalin or formaldehyde, damage from reactive iron, damage from low ionic conditions, damage from high ionic conditions, damage from unbuffered conditions, damage from nucleases, damage from environmental exposure, damage from fire, damage from mechanical stress, damage from enzymatic degradation, damage from microorganisms, damage from preparative mechanical shearing, damage from preparative enzymatic fragmentation, damage having naturally occurred in vivo, damage having occurred during nucleic acid extraction, damage having occurred during sequencing library preparation, damage having been introduced by a polymerase, damage having been introduced during nucleic acid repair, damage having occurred during nucleic acid end-tailing, damage having occurred during nucleic acid ligation, damage having occurred during sequencing, damage having occurred from mechanical handling of DNA, damage having occurred during passage through a nanopore, damage having occurred as part of aging in an organism, damage having occurred as a result if chemical exposure of an individual, damage having occurred by a mutagen, damage having occurred by a carcinogen, damage having occurred by a clastogen, damage having occurred from in vivo inflammation damage due to oxygen exposure, damage due to one or more strand breaks, and any combination thereof.

It is contemplated that nucleic acid material may come from a variety of sources. For example, in some embodiments, nucleic acid material (e.g., comprising one or more double-stranded nucleic acid molecules) is provided from a sample from a human subject, an animal, a plant, a fungi, a virus, a bacterium, a protozoan or any other life form. In other embodiments, the sample comprises nucleic acid material that has been at least partially artificially synthesized. In some embodiments, a sample is or comprises a body tissue, a biopsy, a skin sample, blood, serum, plasma, sweat, saliva, cerebrospinal fluid, mucus, uterine lavage fluid, a vaginal swab, a pap smear, a nasal swab, an oral swab, a tissue scraping, hair, a finger print, urine, stool, vitreous humor, peritoneal wash, sputum, bronchial lavage, oral lavage, pleural lavage, gastric lavage, gastric juice, bile, pancreatic duct lavage, bile duct lavage, common bile duct lavage, gall bladder fluid, synovial fluid, an infected wound, a non-infected wound, an archaeological sample, a forensic sample, a water sample, a tissue sample, a food sample, a bioreactor sample, a plant sample, a bacterial sample, a protozoan sample, a fungal sample, an animal sample, a viral sample, a multi-organism sample, a fingernail scraping, semen, prostatic fluid, vaginal fluid, a vaginal swab, a fallopian tube lavage, a cell free nucleic acid, a nucleic acid within a cell, a metagenomics sample, a lavage or a swab of an implanted foreign body, a nasal lavage, intestinal fluid, epithelial brushing, epithelial lavage, tissue biopsy, an autopsy sample, a necropsy sample, an organ sample, a human identification sample, a non-human identification sample, an artificially produced nucleic acid sample, a synthetic gene sample, a banked or stored nucleic acid sample, tumor tissue, a fetal sample, an organ transplant sample, a microbial culture sample, a nuclear DNA sample, a mitochondrial DNA sample, a chloroplast DNA sample, an apicoplast DNA sample, an organelle sample, and any combination thereof. In some embodiments, the nucleic acid material is derived from more than one source.

As described herein, in some embodiments, it is advantageous to process nucleic acid material so as to improve the efficiency, accuracy, and/or speed of a sequencing process. In some embodiments, the nucleic acid material comprises nucleic acid molecules of a substantially uniform length and/or a substantially known length. In some embodiments, a substantially uniform length and/or a substantially known length is between about 1 and about 1,000,000 bases). For example, in some embodiments, a substantially uniform length and/or a substantially known length may be at least 1; 2; 3; 4; 5; 6; 7; 8; 9; 10; 15; 20; 25; 30; 35; 40; 50; 60; 70; 80; 90; 100; 120; 150; 200; 300; 400; 500; 600; 700; 800; 900; 1000; 1200; 1500; 2000; 3000; 4000; 5000; 6000; 7000; 8000; 9000; 10,000; 15,000; 20,000; 30,000; 40,000; or 50,000 bases in length. In some embodiments, a substantially uniform length and/or a substantially known length may be at most 60,000; 70,000; 80,000; 90,000; 100,000; 120,000; 150,000; 200,000; 300,000; 400,000; 500,000; 600,000; 700,000; 800,000; 900,000; or 1,000,000 bases. By way of specific, non-limiting example, in some embodiments, a substantially uniform length and/or a substantially known length is between about 100 to about 500 bases. In some embodiments, methods described herein comprise steps that target enrich nucleic acid material thereby providing nucleic acid molecules having one or more than one length and/or substantially known lengths. In some embodiments, a nucleic acid material is cut into nucleic acid molecules of a substantially uniform length and/or a substantially known length via one or more targeted endonucleases. In some embodiments, a targeted endonuclease comprises at least one modification.

In some embodiments, a nucleic acid material comprises nucleic acid molecules having a length within one or more substantially known size ranges. In some embodiments, the nucleic acid molecules may be between 1 and about 1,000,000 bases, between about 10 and about 10,000 bases, between about 100 and about 1000 bases, between about 100 and about 600 bases, between about 100 and about 500 bases, or some combination thereof.

In some embodiments, a targeted endonuclease is or comprises at least one of a restriction endonuclease (i.e., restriction enzyme) that cleaves DNA at or near recognition sites (e.g., EcoRI, BamHI, XbaI, HindIII, AluI, AvaII, BsaJI, BstNI, DsaV, Fnu4HI, HaeIII, MaeIII, N1aIV, NSiI, MspJI, FspEI, NaeI, Bsu36I, NotI, HinF1, Sau3AI, PvuIII, SmaI, HgaI, AluI, EcoRV, etc.). Listings of several restriction endonucleases are available both in printed and computer readable forms, and are provided by many commercial suppliers (e.g., New England Biolabs, Ipswich, Mass.). It will be appreciated by one of ordinary skill in the art that any restriction endonuclease may be used in accordance with various embodiments of the present technology. In other embodiments, a targeted endonuclease is or comprises at least one of a ribonucleoprotein complex, such as, for example, a CRISPR-associated (Cas) enzyme/guideRNA complex (e.g., Cas9 or Cpf1) or a Cas9-like enzyme. In other embodiments, a targeted endonuclease is or comprises a homing endonuclease, a zinc-fingered nuclease, a TALEN, and/or a meganuclease (e.g., megaTAL nuclease, etc.), an argonaute nuclease or a combination thereof. In some embodiments, a targeted endonuclease comprises Cas9 or CPF1 or a derivative thereof. In some embodiments, more than one targeted endonuclease may be used (e.g., 2, 3, 4, 5, 6, 7, 8, 9, 10 or more). In some embodiments, a targeted endonuclease may be used to cut at more than one potential target region of a nucleic acid material (e.g., 2, 3, 4, 5, 6, 7, 8, 9, 10 or more). In some embodiments, where there is more than one target region of a nucleic acid material, each target region may be of the same (or substantially the same) length. In some embodiments, where there is more than one target region of a nucleic acid material, at least two of the target regions of known length differ in length (e.g., a first target region with a length of 100 bp and a second target region with a length of 1,000 bp).

In some embodiments, at least one amplifying step includes at least one primer and/or adapter sequence that is or comprises at least one non-standard nucleotide. By way of additional example, in some embodiments, at least one adapter sequence is or comprises at least one non-standard nucleotide. In some embodiments, a non-standard nucleotide is selected from a uracil, a methylated nucleotide, an RNA nucleotide, a ribose nucleotide, an 8-oxo-guanine, a biotinylated nucleotide, a desthiobiotin nucleotide, a thiol modified nucleotide, an acrydite modified nucleotide an iso-dC, an iso dG, a 2′-O-methyl nucleotide, an inosine nucleotide Locked Nucleic Acid, a peptide nucleic acid, a 5 methyl dC, a 5-bromo deoxyuridine, a 2,6-Diaminopurine, 2-Aminopurine nucleotide, an abasic nucleotide, a 5-Nitroindole nucleotide, an adenylated nucleotide, an azide nucleotide, a digoxigenin nucleotide, an I-linker, a 5′ Hexynyl modified nucleotide, an 5-Octadiynyl dU, photocleavable spacer, a non-photocleavable spacer, a click chemistry compatible modified nucleotide, a fluorescent dye, biotin, furan, BrdU, Fluoro-dU, Ioto-dU, and any combination thereof.

In accordance with several embodiments, any of a variety of analytical steps may be used in order to increase one or more of accuracy, speed, and efficiency of a provided process. For example, in some embodiments, sequencing each of the first nucleic acid strand and second nucleic acid strand of a double-stranded nucleic acid molecule includes comparing the sequence of a plurality of strands derived from the first nucleic acid strand to determine a first strand consensus sequence, and comparing the sequence of a plurality of strands derived from the second nucleic acid strand to determine a second strand consensus sequence. In some embodiments, comparing the sequence of the first nucleic acid strand to the sequence of the second nucleic acid strand comprises comparing the first strand consensus sequence and the second strand consensus sequence to provide an error-corrected consensus sequence. In other embodiments, an error-corrected sequence of a double-stranded target nucleic acid molecule can be determined by comparing a single sequence read from a first nucleic acid strand to a single sequence read from a second nucleic acid strand.

One aspect provided by some embodiments, is the ability to generate high quality sequencing information from very small amounts of nucleic acid material. In some embodiments, provided methods and compositions may be used with an amount of starting nucleic acid material of at most about: 1 picogram (pg); 10 pg; 100 pg; 1 nanogram (ng); 10 ng; 100 ng; 200 ng, 300 ng, 400 ng, 500 ng, 600 ng, 700 ng, 800 ng, 900 ng, or 1000 ng. In some embodiments, provided methods and compositions may be used with an input amount of nucleic acid material of at most 1 molecular copy or genome-equivalent, 10 molecular copies or the genome-equivalent thereof, 100 molecular copies or the genome-equivalent thereof, 1,000 molecular copies or the genome-equivalent thereof, 10,000 molecular copies or the genome-equivalent thereof, 100,000 molecular copies or the genome-equivalent thereof, or 1,000,000 molecular copies or the genome-equivalent thereof, For example, in some embodiments, at most 1,000 ng of nucleic acid material is initially provided for a particular sequencing process. For example, in some embodiments, at most 100 ng of nucleic acid material is initially provided for a particular sequencing process. For example, in some embodiments, at most 10 ng of nucleic acid material is initially provided for a particular sequencing process. For example, in some embodiments, at most 1 ng of nucleic acid material is initially provided for a particular sequencing process. For example, in some embodiments, at most 100 pg of nucleic acid material is initially provided for a particular sequencing process. For example, in some embodiments, at most 1 pg of nucleic acid material is initially provided for a particular sequencing process.

As used in this application, the terms “about” and “approximately” are used as equivalents. Any citations to publications, patents, or patent applications herein are incorporated by reference in their entirety. Any numerals used in this application with or without about/approximately are meant to cover any normal fluctuations appreciated by one of ordinary skill in the relevant art.

In various embodiments, enrichment of nucleic acid material, including enrichment of nucleic acid material to region(s) of interest, is provided at a faster rate (e.g., with fewer steps) and with less cost (e.g., utilizing fewer reagents), and resulting in increased desirable data. Various aspects of the present technology have many applications in both pre-clinical and clinical testing and diagnostics as well as other applications.

Specific details of several embodiments of the technology are described below and with reference to the FIGS. 1-22C. Although many of the embodiments are described herein with respect to Duplex Sequencing, other sequencing modalities capable of generating error-corrected sequencing reads, other sequencing modalities for providing sequence information in addition to those described herein are within the scope of the present technology. Additionally, other nucleic acid interrogations are contemplated to benefit from the nucleic acid enrichment methods and reagents described herein. Further, other embodiments of the present technology can have different configurations, components, or procedures than those described herein. A person of ordinary skill in the art, therefore, will accordingly understand that the technology can have other embodiments with additional elements and that the technology can have other embodiments without several of the features shown and described below with reference to the FIGS. 1-22C.

### BRIEF DESCRIPTION OF THE DRAWING

Many aspects of the present disclosure can be better understood with reference to the following drawings. The components in the drawings are not necessarily to scale. Instead, emphasis is placed on illustrating clearly the principles of the present disclosure.

FIG. 1 is a graph plotting a relationship between nucleic acid insert size and resulting family size following amplification in accordance with an embodiment of the present technology.

FIGS. 2A and 2B are schematic illustrating sequencing data generated for different nucleic acid insert sizes in accordance with aspects of the present technology.

FIG. 3 is a schematic illustrating steps of a method for generating targeted fragment sizing with CRISPR/Cas9 in accordance with an embodiment of the present technology. Panel A illustrates gRNA-facilitated binding of Cas9 at targeted DNA sites. Cas9 directed cleavage releases a blunt-ended double-stranded target DNA fragment of known length as shown in Panel B. Panel C depicts a further processing step for positive enrichment/selection of the target DNA fragments via size selection. Optionally, as depicted in Panel D, the enriched DNA fragments can be ligated to adapters for nucleic acid interrogation, such as sequencing.

FIG. 4 is a schematic illustrating steps of a method for generating targeted nucleic acid fragment with known/selected length with a CRISPR/Cas9 variant in accordance with an embodiment of the present technology. Using a CRISPR/Cas9 ribonucleoprotein complex engineered to remain bound to DNA in suitable condition, Panel A illustrates gRNA-facilitated binding of the variant Cas9 to targeted DNA sites. Following cleavage and while Cas9 remains bound to the cleaved 5′ and 3 ends of the target DNA fragment, Panel B illustrates treating the sample with an exonuclease to hydrolyze exposed phosphodiester bonds at exposed 3′ or 5′ ends of DNA. Following negative/enrichment selection of the target DNA fragment via exonuclease destruction of all non-targeted DNA, Cas9 is disassociated from the DNA and releases a blunt-ended double-stranded target DNA fragment of known length as shown in Panel C. Panel D depicts an optional further processing step for positive enrichment/selection of the target DNA fragments via size selection. Optionally, as depicted in Panel E, the enriched DNA fragments can be ligated to adapters for nucleic acid interrogation, such sequencing.

FIG. 5 is a schematic illustrating steps of a method for generating targeted nucleic acid fragment with known/selected length with a CRISPR/Cas9 variant in accordance with another embodiment of the present technology. Panel A illustrates using a CRISPR/Cas9 ribonucleoprotein complex engineered to remain bound to DNA in suitable condition, wherein the ribonucleoprotein complex comprises a capture label. Guide RNA (gRNA)-facilitated binding of the variant Cas9 ribonucleoprotein complex with capture label is followed by cleavage of the double-stranded target DNA. Following cleavage and while Cas9 remains bound to the cleaved 5′ and 3 ends of the target DNA fragment, Panel B illustrates treating the sample with an exonuclease to hydrolyze exposed phosphodiester bonds at exposed 3′ or 5′ ends of DNA. Following negative/enrichment selection of the target DNA fragment via exonuclease destruction of all non-targeted DNA, and while Cas9 remains bound, Panel C illustrates a positive enrichment/selection process of target nucleic acid capture involving the step-wise addition of functionalized surfaces that are capable of binding the capture label associated with the ribonucleoprotein complex as it remains bound to the target nucleic acid. After the affinity-based enrichment step, and as depicted in Panel D, Cas9 is disassociated from the DNA and releases a blunt-ended double-stranded target DNA fragment of known length. Panel E depicts an optional further processing step for positive enrichment/selection of the target DNA fragments via size selection. Optionally, as depicted in Panel F, the enriched DNA fragments can be ligated to adapters for nucleic acid interrogation, such sequencing.

FIG. 6 is a schematic illustrating steps of a method for generating targeted nucleic acid fragment with known/selected length with a catalytically inactive variant of Cas9 in accordance with an embodiment of the present technology. Using a catalytically inactive Cas9 ribonucleoprotein complex engineered to target and bind double-stranded DNA, Panel A illustrates gRNA-facilitated binding of the variant Cas9 to targeted DNA sites. Following binding, Panel B illustrates treating the sample with an exonuclease to hydrolyze exposed phosphodiester bonds at exposed 3′ or 5′ ends of DNA. The catalytically inactive variant of Cas9 does not cut the target DNA but provides exonuclease resistance such that exonuclease activity cleaves each nucleotide base until blocked by the bound Cas9 complex. Following negative/enrichment selection of the target DNA fragment via exonuclease destruction of all non-targeted DNA, catalytically inactive Cas9 is disassociated from the DNA and releases a double-stranded target DNA fragment of known length as shown in Panel C. Panel D depicts an optional further processing step for positive enrichment/selection of the target DNA fragments via size selection. Optionally, as depicted in Panel E, the enriched DNA fragments can be ligated to adapters for nucleic acid interrogation, such sequencing.

FIG. 7 is a schematic illustrating steps of a method for generating targeted fragment sizing with a catalytically inactive variant of Cas9 in accordance with another embodiment of the present technology. Panel A illustrates using a catalytically inactive variant of Cas9 in a ribonucleoprotein complex engineered to remain bound to DNA in suitable condition, and wherein the ribonucleoprotein complex comprises a capture label. Guide RNA (gRNA)-facilitated binding of the catalytically inactive variant Cas9 ribonucleoprotein complex with capture label is followed by addition of an exonuclease to the sample to hydrolyze exposed phosphodiester bonds at exposed 3′ or 5′ ends of DNA. The catalytically inactive variant of Cas9 does not cut the target DNA but provides exonuclease resistance such that exonuclease activity cleaves each nucleotide base until blocked by the bound Cas9 complex. Following negative/enrichment selection of the target DNA fragment via exonuclease destruction of all non-targeted DNA, and while catalytically inactive Cas9 remains bound, Panel C illustrates a positive enrichment/selection process of target nucleic acid capture involving the step-wise addition of functionalized surfaces that are capable of binding the capture label associated with the ribonucleoprotein complex as it remains bound to the target nucleic acid. After the affinity-based enrichment step, and as depicted in Panel D, Cas9 is disassociated from the DNA and releases a double-stranded target DNA fragment of known length. Panel E depicts an optional further processing step for positive enrichment/selection of the target DNA fragments via size selection. Optionally, as depicted in Panel F, the enriched DNA fragments can be ligated to adapters for nucleic acid interrogation, such sequencing.

FIG. 8 is a schematic illustrating a target nucleic acid enrichment scheme using both catalytically active and catalytically inactive Cas9 in accordance with another embodiment of the technology. Both catalytically active and catalytically inactive Cas9 ribonucleoprotein complexes can be targeted to desired sequences in a sample. Catalytically active Cas 9 ribonucleoprotein complexes are directed to regions flanking a target DNA region and are used to cleave target double-stranded DNA to release a blunt-ended double-stranded target DNA fragment of known length. One or more catalytically inactive ribonucleoprotein complexes bearing a capture label are directed to target sequence regions between the two site selected cleavage sites. Following cleavage of target DNA to release the DNA fragment, addition of functionalized surfaces that are capable of binding a capture label associated with the catalytically inactive ribonucleoprotein complex can facilitate positive enrichment/selection of the target fragment.

FIGS. 9A and 9B are conceptual illustrations of methods steps for positive enrichment/selection of target nucleic acid fragments using a catalytically inactive variant of Cas 9 ribonucleoprotein complex bearing a capture label in accordance with an embodiment of the present technology. Fragmented double-stranded DNA fragments in a sample (e.g., mechanically sheared, acoustically fragmented, cell free DNA, etc.) can be positively enriched/selected via target directed binding by a catalytically inactive Cas9 ribonucleoprotein complex in solution (FIG. 9A). Step-wise addition of functionalized surfaces that are capable of binding the capture label associated with the ribonucleoprotein complex as it remains bound to the target nucleic acid facilitate pull-down (e.g., affinity purification) of the desired double-stranded DNA fragment while discarding non targeted fragments (FIG. 9B).

FIG. 10 is a schematic illustrating methods steps for positive enrichment/selection of target nucleic acid fragments using a catalytically inactive variant of Cas 9 ribonucleoprotein complex bearing a capture label in accordance with an embodiment of the present technology. Panel A illustrates a plurality of fragmented double-stranded DNA fragments of varying size in a sample, including Molecule 2 which is too small to reliably enrich via size selection or affinity-based methods. Panel B illustrates ligating adapters to the 5′ and 3′ ends of the molecules in the sample, thereby making such DNA fragments longer in length. Panel C illustrates a positive enrichment/selection step of molecule 2 via target directed binding by a catalytically inactive Cas9 ribonucleoprotein complex bearing a capture label in solution followed by affinity purification by pull-down method.

FIG. 11 is a schematic illustrating steps of a method for enriching targeted nucleic acid material using a negative enrichment scheme (Panel A) and a positive enrichment scheme (Panel B) in accordance with an embodiment of the present technology. Panel A shows ligation of hairpin adapters to the 5′ and 3′ ends of a double-stranded target DNA molecule to generate adapter-nucleic acid complexes with no exposed ends. The adapter-nucleic acid complexes are treated with exonuclease in a negative enrichment/selection scheme to eliminate nucleic acid material fragments and adapters with unprotected 5′ and 3′ ends (e.g., adapter-nucleic acid complexes without 4 ligated phosphodiester bonds, unligated DNA, single stranded nucleic acid material, free adapters, etc.) as illustrated on the right side of Panel B. Exonuclease resistant adapter-nucleic acid complexes can be further enriched via size selection or via target sequence (e.g., CRISPR/Cas9 pull-down) (Panel B, left side). Desired adapter-target nucleic acid complexes can be further processed via amplification and/or sequencing.

FIG. 12 illustrates an embodiment in which hairpin adapters bearing a capture label are ligated to target double-stranded DNA for affinity-based enrichment, and in accordance with another embodiment of the present technology.

FIG. 13 is a schematic illustrating method steps for positive enrichment of an adapter-target nucleic acid complex using hairpin adapters (Panel A) followed by rolling circle amplification (Panels B and C) and amplicon-making steps for generating amplicons of a first and second strand of a double-stranded nucleic acid fragment in substantially the same ratio (Panel D) in accordance with an embodiment of the present technology.

FIG. 14 is a schematic illustrating steps of a method for generating targeted nucleic acid fragments with known/selected length with different 5′ and 3′ ligatable ends comprising single-stranded overhang regions with known nucleotide length and sequence with CRISPR/Cpf1 in accordance with an embodiment of the present technology. Panel A illustrates gRNA-facilitated binding of Cpf1 at a targeted DNA site. Cpf1 directed cleavage generates a staggered cut providing a 4 (depicted) or 5 nucleotide overhang (e.g., “sticky end”). Site directed Cpf1 cleavage flanking a target DNA sequence, generates a double-stranded target DNA fragment of known length (e.g., which can be enriched via size selection) with sticky end 1 at the 5′ end and sticky end 2 at the 3′ end of the fragment (Panel B). Panel B further illustrates attaching adapter 1 at the 5′ end and adapter 2 at the 3′ end of the fragment, wherein adapters 1 and 2 comprise at least partially complementary overhang sequences to sticky ends 1 and 2 on the fragment, respectively.

FIG. 15 is a schematic illustrating steps of a method for affinity-based enrichment of a target DNA fragment comprising sticky end(s) (e.g., such as target DNA fragments generated in the method of FIG. 14) in accordance with an embodiment of the present technology. Panel A illustrates step-wise addition of a functionalized surface that is capable of binding a sticky end associated with the cut target DNA fragment in solution. Once bound to the functionalized surface, the affinity interaction facilitates pull-down (e.g., affinity purification) of the desired double-stranded DNA fragment while discarding non targeted fragments as shown in Panel B.

FIG. 16 is a schematic illustrating steps of a method for affinity-based enrichment of a target DNA fragment comprising sticky end(s) (e.g., such as target DNA fragments generated in the method of FIG. 14) in accordance with another embodiment of the present technology. Panel A illustrates step-wise addition of a capture label-bearing oligonucleotide having a nucleotide sequence at least partially complementary to at a portion of a sticky end associated with the cut target DNA fragment in solution. As shown in Panel B, further addition of a functionalized surface that is capable of binding the capture label facilitates pull-down (e.g., affinity purification) of the desired double-stranded DNA fragment while discarding non targeted fragments.

FIG. 17 is a schematic illustrating steps of a method for targeted fragment enrichment of nucleic acid material having a known length and having different 5′ and 3′ ligatable ends comprising long single-stranded overhang regions with known nucleotide length and sequence using Cas9 Nickase and in accordance with an embodiment of the present technology. Panel A illustrates gRNA targeted binding of paired Cas9 nickases in a targeted DNA region. Double-strand breaks can be introduced through the use of paired nickases to excise the target DNA region and when paired Cas9 nickases are used, long overhangs (sticky ends 1 and 2) are produced on each of the cleaved ends instead of blunt ends as illustrated in Panel B. Panel C illustrates step-wise addition of a functionalized surface that is capable of binding a long sticky end (e.g., sticky end 1) associated with the cut target DNA fragment in solution. Once bound to the functionalized surface, the affinity interaction facilitates pull-down (e.g., affinity purification) of the desired double-stranded DNA fragment while discarding non targeted fragments as shown in Panel D. Panel E illustrates a variation of a positive enrichment step comprising addition of a capture label-bearing oligonucleotide having a nucleotide sequence at least partially complementary to at a portion of a long sticky end (e.g., sticky end 1) associated with the cut target DNA fragment in solution. Panel F illustrates annealing of a second oligo strand at least partially complementary to a portion of the capture label-bearing oligonucleotide. Enzymatic extension of the second oligo strand and ligation to the template DNA fragment generates an adapter-target DNA complex. Further steps can include introduction of a functionalized surface (not shown) that is capable of binding the capture label to facilitate pull-down (e.g., affinity purification) of the desired adapter-double-stranded DNA complex while discarding non targeted fragments.

FIG. 18 is a schematic illustrating a target nucleic acid enrichment scheme using catalytically inactive Cas9 in accordance with another embodiment of the present technology. Catalytically inactive Cas9 ribonucleoprotein complexes can be targeted to desired sequences in a sample. One or more catalytically inactive ribonucleoprotein complexes bearing one or more capture labels directs other protein complex structures to the target DNA region. Where the protein complex structure covers the target DNA region, exonuclease resistance is provided. Following treatment with an exonuclease or a combination of endonucleases and exonucleases, affinity purification of the protein complex (e.g., via a capture label binding to a functionalized surface, antibody pull-down, etc.), the target nucleic acid fragment can be released from ribonucleotide complex binding.

FIGS. 19A and 19B are conceptual illustrations of a prepared DNA library and reagents that can be used as a tool to selectively interrogate DNA regions of interest in accordance with an embodiment of the present technology. Uniquely tagged catalytically inactive Cas9 is target directed to multiple (e.g., interspaced) regions of isolated/unfragmented genomic DNA (or other large fragments of DNA) (FIG. 19A). Each catalytically inactive Cas9 ribonucleoprotein comprises a known oligonucleotide tag with known sequence (e.g., a code sequence) and is bound to a pre-designed region of a genome. When using the DNA library, a user can step-wise add one or more probes comprising the compliment of the code sequence corresponding to the region of the genome of interest (e.g., an anticode sequence). A method of fragmentation can be used to fragment the genomic DNA in various sizes (e.g., restriction enzymatic digestion, mechanical shearing, etc.). The probes comprise a capture label affixed or incorporated thereto (FIG. 19B). Addition of a functionalized surface that is capable of binding the capture label can be added for affinity purification and positive enrichment of the desired genomic region for interrogation.

FIG. 20 illustrates a step of a method for affinity-based enrichment and sequencing of a target DNA fragment for use with a direct digital sequencing method in accordance with an embodiment of the present technology. Panel A shows selected adapter attachment to a target DNA fragment comprising sticky end(s) (e.g., such as target DNA fragments generated in the method of FIG. 14 or FIG. 17). Panel A further illustrates attaching adapter 1 at the 5′ end and adapter 2 at the 3′ end of the fragment, wherein adapters 1 and 2 comprise at least partially complementary overhang sequences to sticky ends 1 and 2 on the fragment, respectively. Adapter 1 has a Y-shape and comprises 5′ and 3′ single-stranded arms bearing different labels (A and B) comprising different properties. Adapter 2 is a hairpin-shaped adapter. Panel B illustrates a step in a direct digital sequencing method where label A is configured to be bound to a functional surface. Label B provides a physical property (e.g., electric charge, magnetic property, etc.) such that application of an electrical or magnetic field causes denaturation of the first and second strands of the double-stranded adapter-DNA complex followed by electro-stretching of the DNA fragment. The first and second strands remain tethered by the hairpin adapter such that sequence information from the enriched/targeted strand provides duplex sequence information for error-correction and other nucleic acid interrogation (e.g., assessment of DNA damage, etc.).

FIG. 21 illustrates a step of a method for affinity-based enrichment for sequencing of a target DNA fragment using a direct digital sequencing method in accordance with another embodiment of the present technology. Panel A shows affinity-based enrichment of a target DNA fragment comprising sticky end(s) (e.g., such as target DNA fragments generated in the method of FIG. 14 or FIG. 17). As illustrated, a hairpin adapter has been attached to a 3′ end of the double-stranded DNA fragment in a sequence-dependent manner. The target DNA molecule(s) can be flowed over a functionalized surface capable of binding a sticky end associated with the cut target DNA fragment (e.g., having bound oligonucleotides). Additionally, a second oligonucleotide strand comprising label B and at least partially complementary to a portion of the bound oligonucleotide is added into solution. Annealing and ligation of the adapter/DNA fragment components provides an adapter-target double-stranded DNA complex bound to a surface suitable for direct digital sequencing (Panel B). Application of an electrical or magnetic field and electro-stretching of the adapter-DNA complex for sequencing steps can occur as described, for example, in FIG. 20.

FIG. 22A illustrates a nucleic acid adapter molecule for use with some embodiments of the present technology and a double-stranded adapter-nucleic acid complex resulting from ligation of the adapter molecule to a double-stranded nucleic acid fragment in accordance with an embodiment of the present technology.

FIGS. 22B and 22C are conceptual illustrations of various Duplex Sequencing method steps in accordance with an embodiment of the present technology.

### DEFINITIONS

In order for the present disclosure to be more readily understood, certain terms are first defined below. Additional definitions for the following terms and other terms are set forth throughout the specification.

In this application, unless otherwise clear from context, the term “a” may be understood to mean “at least one.” As used in this application, the term “or” may be understood to mean “and/or.” In this application, the terms “comprising” and “including” may be understood to encompass itemized components or steps whether presented by themselves or together with one or more additional components or steps. Where ranges are provided herein, the endpoints are included. As used in this application, the term “comprise” and variations of the term, such as “comprising” and “comprises,” are not intended to exclude other additives, components, integers or steps.

About: The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Biological Sample: As used herein, the term “biological sample” or “sample” typically refers to a sample obtained or derived from a biological source (e.g., a tissue or organism or cell culture) of interest, as described herein. In some embodiments, a source of interest comprises an organism, such as an animal or human. In other embodiments, a source of interest comprises a microorganism, such as a bacterium, virus, protozoan, or fungus. In further embodiments, a source of interest may be a synthetic tissue, organism, cell culture, nucleic acid or other material. In yet further embodiments, a source of interest may be a plant-based organism. In yet another embodiment, a sample may be an environmental sample such as, for example, a water sample, soil sample, archeological sample, or other sample collected from a non-living source. In other embodiments, a sample may be a multi-organism sample (e.g., a mixed organism sample). In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; pap smear, oral swabs; nasal swabs; washings or lavages such as a ductal lavages or broncheoalveolar lavages; vaginal fluid, aspirates; scrapings; bone marrow specimens; tissue biopsy specimens; fetal tissue or fluids; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In a particular embodiment, a biological sample is a liquid biopsy obtained from a subject. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Capture label: As used herein, the term “capture label” “(which may also be referred to as a “capture tag”, “capture moiety”, “affinity label”, “affinity tag”, “epitope tag”, “tag”, “prey” moiety or chemical group, among other names) refers to a moiety that can be integrated into, or onto, a target molecule, or substrate, for the purposes of purification. In some embodiments, the capture label is selected from a group comprising a small molecule, a nucleic acid, a peptide, or any uniquely bindable moiety. In some embodiments, the capture label is affixed to the 5′ of a nucleic acid molecule. In some embodiments, the capture label is affixed to the 3′ of a nucleic acid molecule. In some embodiments, the capture label is conjugated to a nucleotide within the internal sequence of a nucleic acid molecule not at either end. In some embodiments, the capture label is a sequence of nucleotides within the nucleic acid molecule. In some embodiments, the capture label is selected from a group of biotin, biotin deoxythymidine dT, biotin NHS, biotin TEG, desthiobiotin NHS, digoxigenin NHS, DNP TEG, thiols, among others. In some embodiments, capture labels include, without limitation, biotin, avidin, streptavidin, a hapten recognized by an antibody, a particular nucleic acid sequence and magnetically attractable particles. In some embodiments, chemical modification (e.g., Acridite™-modified, adenylated, azide-modified, alkyne-modified, I-Linker™-modified etc.) of nucleic acid molecules can serve as a capture label.

Cut site: Also called “cleavage site” and “nick site”, is the bond, or pair of bonds between nucleotides in a nucleic acid molecule. In the case of double stranded nucleic acid molecules, such as double stranded DNA, the cut site can entail bonds (commonly phosphodiester bonds) which are immediately adjacent from each other in a double stranded molecule such that after cutting a “blunt” end is formed. The cut site can also entail two nucleotide bonds that are on each single strand of the pair that are not immediately opposite from each other such that when cleaved a “sticky end” is left, whereby regions of single stranded nucleotides remain at the terminal ends of the molecules. Cut sites can be defined by particular nucleotide sequence that is capable of being recognized by an enzyme, such as a restriction enzyme, or another endonuclease with sequence recognition capability such as CRISPER/Cas9. The cut site may be within the recognition sequence of such enzymes (i.e. type 1 restriction enzymes) or adjacent to them by some defined interval of nucleotides (i.e. type 2 restriction enzymes). Cut sites can also be defined by the position of modified nucleotides that are capable of being recognized by certain nucleases. For example, abasic sites can be recognized and cleaved by endonuclease VII as well as the enzyme FPG. Uracil based can be recognized and rendered into abasic sites by the enzyme UDG. Ribose-containing nucleotides in an otherwise DNA sequence can be recognized and cleaved by RNAseH2 when annealed to complementary DNA sequences.

Determine: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Extraction moiety: As used herein the term “extraction moiety” (which may also be referred to as a “binding partner”, an “affinity partner”, a “bait” moiety or chemical group among other names) refers to an isolatable moiety or any type of molecule that allows affinity separation of nucleic acids bearing the capture label from nucleic acids lacking the capture label. In some embodiments, the extraction moiety is selected from a group comprising a small molecule, a nucleic acid, a peptide, an antibody or any uniquely bindable moiety. The extraction moiety can be linked or linkable to a solid phase or other surface for forming a functionalized surface. In some embodiments, the extraction moiety is a sequence of nucleotides linked to a surface (e.g., a solid surface, bead, magnetic particle, etc.). In some embodiments, the extraction moiety is selected from a group of avidin, streptavidin, an antibody, a polyhistadine tag, a FLAG tag or any chemical modification of a surface for attachment chemistry. Non-limiting examples of these latter include azide and alkyne groups which can form 1,2,3-triazole bonds via “Click” methods, or thiol an azide and terminal alkyne, thiol-modified surfaces can covalently react with Acrydite-modified oligonucleotides and aldehyde and ketone modified surfaces which can react to affix I-Linker™ labeled oligonucleotides.

Functionalized surface: As used herein, the term “functionalized surface” refers to a solid surface, a bead, or another fixed structure that is capable of binding or immobilizing a capture label. In some embodiments, the functionalized surface comprises an extraction moiety capable of binding a capture label. In some embodiments, an extraction moiety is linked directly to a surface. In some embodiments, chemical modification of the surface functions as an extraction moiety. In some embodiments, a functionalized surface can comprise controlled pore glass (CPG), magnetic porous glass (MPG), among other glass or non-glass surfaces. Chemical functionalization can entail ketone modification, aldehyde modification, thiol modification, azide modification, and alkyne modifications, among others. In some embodiments, the functionalized surface and an oligonucleotide used for adapter synthesis are linked using one or more of a group of immobilization chemistries that form amide bonds, alkylamine bonds, thiourea bonds, diazo bonds, hydrazine bonds, among other surface chemistries. In some embodiments, the functionalized surface and an oligonucleotide used for adapter synthesis are linked using one or more of a group of reagents including EDAC, NHS, sodium periodate, glutaraldehyde, pyridyl disulfides, nitrous acid, biotin, among other linking reagents.

gRNA: As used herein, “gRNA” or “guide RNA”, refers to short RNA molecules which include a scaffold sequence suitable for a targeted endonuclease (e.g., a Cas enzyme such as Cas9 or Cpf1 or another ribonucleoprotein with similar properties, etc.) binding to a substantially target-specific sequence which facilitates cutting of a specific region of DNA or RNA.

Nucleic acid: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present technology. Alternatively, or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, hexose or Locked Nucleic acids) as compared with those in commonly occurring natural nucleic acids. In some embodiments, a nucleic acid has a nucleotide sequence that encodes a functional gene product such as an RNA or protein. In some embodiments, a nucleic acid includes one or more introns. In some embodiments, a nucleic acid may be a non-protein coding RNA product, such as a microRNA, a ribosomal RNA, or a CRISPER/Cas9 guide RNA. In some embodiments, a nucleic acid serves a regulatory purpose in a genome. In some embodiments, a nucleic acid does not arise from a genome. In some embodiments, a nucleic acid includes intergenic sequences. In some embodiments, a nucleic acid derives from an extrachromosomal element or a non-nuclear genome (mitochondrial, chloroplast etc.), In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 2, 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 200, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double-stranded. In some embodiments a nucleic acid has a nucleotide sequence comprising at least one element that encodes, or is the complement of a sequence that encodes, a polypeptide. In some embodiments, a nucleic acid has enzymatic activity. In some embodiments the nucleic acid serves a mechanical function, for example in a ribonucleoprotein complex or a transfer RNA. In some embodiments a nucleic acid function as an aptamer. In some embodiments a nucleic acid may be used for data storage. In some embodiments a nucleic acid may be chemically synthesized in vitro.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Single Molecule Identifer (SMI): As used herein, the term “single molecule identifier” or “SMI”, (which may be referred to as a “tag” a “barcode”, a “Molecular bar code”, a “Unique Molecular Identifier”, or “UMI”, among other names) refers to any material (e.g., a nucleotide sequence, a nucleic acid molecule feature) that is capable of distinguishing an individual molecule in a large heterogeneous population of molecules. In some embodiments, a SMI can be or comprise an exogenously applied SMI. In some embodiments, an exogenously applied SMI may be or comprise a degenerate or semi-degenerate sequence. In some embodiments substantially degenerate SMIs may be known as Random Unique Molecular Identifiers (R-UMIs). In some embodiments an SMI may comprise a code (for example a nucleic acid sequence) from within a pool of known codes. In some embodiments pre-defined SMI codes are known as Defined Unique Molecular Identifiers (D-UMIs). In some embodiments, a SMI can be or comprise an endogenous SMI. In some embodiments, an endogenous SMI may be or comprise information related to specific shear-points of a target sequence, or features relating to the terminal ends of individual molecules comprising a target sequence. In some embodiments an SMI may relate to a sequence variation in a nucleic acid molecule cause by random or semi-random damage, chemical modification, enzymatic modification or other modification to the nucleic acid molecule. In some embodiments the modification may be deamination of methylcytosine. In some embodiments the modification may entail sites of nucleic acid nicks. In some embodiments, an SMI may comprise both exogenous and endogenous elements. In some embodiments an SMI may comprise physically adjacent SMI elements. In some embodiments SMI elements may be spatially distinct in a molecule. In some embodiments an SMI may be a non-nucleic acid. In some embodiments an SMI may comprise two or more different types of SMI information. Various embodiments of SMIs are further disclosed in International Patent Publication No. WO2017/100441, which is incorporated by reference herein in its entirety.

Strand Defining Element (SDE): As used herein, the term “Strand Defining Element” or “SDE”, refers to any material which allows for the identification of a specific strand of a double-stranded nucleic acid material and thus differentiation from the other/complementary strand (e.g., any material that renders the amplification products of each of the two single stranded nucleic acids resulting from a target double-stranded nucleic acid substantially distinguishable from each other after sequencing or other nucleic acid interrogation). In some embodiments, a SDE may be or comprise one or more segments of substantially non-complementary sequence within an adapter sequence. In particular embodiments, a segment of substantially non-complementary sequence within an adapter sequence can be provided by an adapter molecule comprising a Y-shape or a “loop” shape. In other embodiments, a segment of substantially non-complementary sequence within an adapter sequence may form an unpaired “bubble” in the middle of adjacent complementary sequences within an adapter sequence. In other embodiments an SDE may encompass a nucleic acid modification. In some embodiments an SDE may comprise physical separation of paired strands into physically separated reaction compartments. In some embodiments an SDE may comprise a chemical modification. In some embodiments an SDE may comprise a modified nucleic acid. In some embodiments an SDE may relate to a sequence variation in a nucleic acid molecule caused by random or semi-random damage, chemical modification, enzymatic modification or other modification to the nucleic acid molecule. In some embodiments the modification may be deamination of methylcytosine. In some embodiments the modification may entail sites of nucleic acid nicks. Various embodiments of SDEs are further disclosed in International Patent Publication No. WO2017/100441, which is incorporated by reference herein in its entirety.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

### DETAILED DESCRIPTION

The present technology relates generally to methods for enrichment of nucleic acid material for sequencing applications and other nucleic acid material interrogations and associated reagents for use in such methods. Some embodiments of the technology are directed to enriching one or more regions of interest within the nucleic acid material for sequencing applications such as Duplex Sequencing applications and other sequencing applications for achieving high accuracy sequencing reads. For example, various embodiments of the present technology include selectively enriching nucleic acid material (e.g., genomic DNA material) for regions of interest and performing Duplex Sequencing methods to provide an error-corrected sequence read of the enriched nucleic acid material. Further examples of the present technology are directed to methods for performing Duplex Sequencing methods or other sequencing methods (e.g., single consensus sequencing methods, Hyb & Seq™ sequencing methods, nanopore sequencing methods, etc.) on nucleic acid material enriched for regions of interest. In various embodiments, enrichment of nucleic acid material, including enrichment of nucleic acid material to region(s) of interest, is provided at a faster rate (e.g., with fewer steps) and with less cost (e.g., utilizing fewer reagents), and resulting in increased desirable data. Various aspects of the present technology have many applications in both pre-clinical and clinical testing and diagnostics as well as other applications.

Duplex Sequencing (DS) is a method for producing error-corrected nucleic acid sequence reads from double-stranded nucleic acid molecules. In certain aspects of the technology, DS can be used to independently sequence both strands of individual nucleic acid molecules in such a way that the derivative sequence reads can be recognized as having originated from the same double-stranded nucleic acid parent molecule during massively parallel sequencing, but also differentiated from each other as distinguishable entities following sequencing. The resulting sequence reads from each strand are then compared for the purpose of obtaining an error-corrected sequence of the original double-stranded nucleic acid molecule, known as a Duplex Consensus Sequence. The process of DS makes it possible to confirm whether one or both strands of an original double-stranded nucleic acid molecule are represented in the generated sequencing data used to form a Duplex Consensus Sequence.

The error rate of standard next-generation sequencing is on the approximate order of 1/100- 1/1000 and when fewer than 1/100- 1/1000 of the molecules carry a sequence variant, the presence of it is obscured by the background error rate of the sequencing process. DS, on the other hand can accurately detect extremely low frequency variants due to the high degree of error correction obtained. The high degree of error correction provided by the strand-comparison technology of DS reduces sequencing errors of double-stranded nucleic acid molecules by multiple orders of magnitude as compared with standard next-generation sequencing methods. This reduction in errors improves the accuracy of sequencing in nearly all types of sequences but can be particularly well suited to biochemically challenging sequences that are well known in the art to be particularly error prone or where the molecular population being sequenced is heterogeneous (i.e. a minor subset of the molecules carries a sequence variant that others do not). One non-limiting example of such type of sequence is homopolymers or other microsatellites/short-tandem repeats. Another non-limiting example of error prone sequences that benefit from DS error correction are molecules that have been damaged, for example, by heating, radiation, mechanical stress, or a variety of chemical exposures which creates chemical adducts that are error prone during copying by one or more nucleotide polymerases and also those that create single-stranded DNA at ends of molecules or as nicks and gaps. In highly damaged DNA (oxidation, deamination, etc.), which occur through fixation processes (i.e. FFPE in clinical pathology) or ancient DNA or in forensic applications where material has been exposed to harsh chemicals or environments, Duplex Sequencing is particularly useful to reduce the high resulting level of error that damage confers.

In further embodiments, DS can also be used for the accurate detection of minority sequence variants among a population of double-stranded nucleic acid molecules. One non-limiting example of this application is detection of a small number of DNA molecules derived from a cancer, among a larger number of unmutated molecules from non-cancerous tissues within a subject. DS is also well suited for accurate genotyping of difficult-to-sequence regions of the genome (homopolymers, microsatellites, G-tetraplexes etc.) where the error rate of standard sequencing is especially high. Another non-limiting application for rare variant detection by DS is early detection of DNA damage resulting from genotoxin exposure. A further non-limiting application of DS is for detection of mutations generated from either genotoxic or non-genotoxic carcinogens by looking at genetic clones that are emerging with driver mutations. A yet further non-limiting application for accurate detection of minority sequence variants is to generate a mutagenic signature associated with a genotoxin. Additional non-limiting examples of the utility of DS can be found in Salk et al, Nature Reviews Genetics 2018, PMID 29576615, which is incorporated by reference herein its entirety.

Various embodiments pertaining to enrichment of nucleic acid material for sequencing applications as well as other nucleic acid material interrogations have utility in single molecule sequencing applications and direct digital sequencing methods. In some embodiments, technology using single molecule hybridization with barcoded probes may be used to characterize and/or quantify a genomic region. In general, such technology uses molecular “barcodes” and single molecule imaging to detect and count specific nucleic acid targets in a single reaction without amplification. Typically, each color-coded barcode is attached to a single target-specific probe corresponding to a genomic region of interest. Mixed together with controls, they form a multiplexed Code Set. In some embodiments, two probes are used to hybridize each individual target nucleic acid. In particular arrangements, a Reporter Probe carries the signal and a Capture Probe allows the complex to be immobilized for data collection. After hybridization, the excess probes are removed, and the immobilized probe/target complexes may be analyzed by a digital analyzer for data collection. Color codes are counted and tabulated for each target molecule (e.g., a genomic region of interest). Suitable digital analyzers include nCounter® Analysis System (NanoString™ Technologies; Seattle, Wash.). Methods and reagents including molecular “barcodes”, and apparatus suitable for NanoString™ technology are further described, for example, in U.S. Patent Pub. Nos. 2010/0112710, 2010/0047924, 2010/0015607, the entire contents of each are herein incorporated by reference.

Direct Digital Sequencing (DDS) technology includes methods for providing highly accurate single molecule sequencing that simultaneously captures and directly sequences DNA and RNA for a variety of research, diagnostic and other applications. DDS provides both short and long sequencing reads without library creation or amplification steps, and is described in, for example, in International Patent Publication No. WO 2016/081740, which is incorporated by reference herein. In general, direct sequencing of nucleic acid targets is achieved by hybridization of fluorescent molecular barcodes onto the native nucleic acid targets. As further described in U.S. Pat. No. 7,919,237 and as available from NanoString™ Technologies, Inc. (Seattle, Wash.), oligomers that are extensions of targeting nucleotide sequences are stretched by an electro-stretching technique spatially separating the monomers wherein each monomer is connected to a unique label. Thus, the pattern of labeled monomers can be used to identify the barcode on the oligomeric tag.

Additionally, various embodiments pertaining to enrichment of nucleic acid material have utility in other forms of characterization and/or quantification of nucleic acid material are known in the art. For example, characterization of nucleic acid material to determine the presence or absence of genomic mutations, DNA variants, quantification of DNA or RNA copy number, and other applications may benefit from selective enrichment of target nucleic acid material as provided herein. Examples of some methodologies include, but are not limited to, single molecule sequencing (e.g., single molecule real-time sequencing, nanopore sequencing, high-throughput sequencing or Next Generation Sequencing (NGS), etc.), digital PCR, bridge PCR, emulsion PCR, semiconductor sequencing, among others. One of ordinary skill in the art will recognize other nucleic acid interrogation methods and technology that may be suitably used to interrogate and/or benefit from enriched nucleic acid material.

Methods incorporating DS, as well as other sequencing modalities may include ligation of one or more sequencing adapters to a target double-stranded nucleic acid molecule to produce a double-stranded target nucleic acid complex. Such adapter molecules may include one or more of a variety of features suitable for MPS platforms such as, for example, sequencing primer recognition sites, amplification primer recognition sites, barcodes (e.g., single molecule identifier (SMI) sequences, indexing sequences, single-stranded portions, double-stranded portions, strand distinguishing elements or features, and the like. The use of highly pure sequencing adapters for DS, or any next-generation sequencing technology, is important for obtaining reproducible data of high quality and maximizing sequence yield of a sample (i.e., the relative percentage of inputted molecules that are converted to independent sequence reads). It is particularly important with DS because of the need to successfully recover both strands of the original duplex molecules.

With regard to the efficiency of a DS process or other high-accuracy sequencing modality, two types of efficiency are further described herein: conversion efficiency and workflow efficiency. For the purposes of discussing efficiency of DS, conversion efficiency can be defined as the fraction of unique nucleic acid molecules inputted into a sequencing library preparation reaction from which at least one duplex consensus sequence read is produced. Workflow efficiency may relate to relative inefficiencies with the amount of time, relative number of steps and/or financial cost of reagents/materials needed to carry out these steps to produce a Duplex Sequencing library and/or carry out targeted enrichment for sequences of interest.

In some instances, either or both conversion efficiency and workflow efficiency limitations may limit the utility of high-accuracy DS for some applications where it would otherwise be very well suited. For example, a low conversion efficiency would result in a situation where the number of copies of a target double-stranded nucleic acid is limited, which may result in a less than desired amount of sequence information produced. Non-limiting examples of this concept include DNA from circulating tumor cells or cell-free DNA derived from tumors, or prenatal infants that are shed into body fluids such as plasma and intermixed with an excess of DNA from other tissues. Although DS typically has the accuracy to be able to resolve one mutant molecule among more than one hundred thousand unmutated molecules, if only 10,000 molecules are available in a sample, for example, and even with the ideal efficiency of converting these to duplex consensus sequence reads being 100%, the lowest mutation frequency that could be measured would be 1/(10,000*100%)= 1/10,000. As a clinical diagnostic, having maximum sensitivity to detect the low-level signal of a cancer or a therapeutically-relevant mutation can be important and so a relatively low conversion efficiency would be undesirable in this context. Similarly, in forensic applications, often very little DNA is available for testing. When only nanogram or picogram quantities can be recovered from a crime scene or site of a natural disaster, and where the DNA from multiple individuals is mixed together, having maximum conversion efficiency can be important in being able to detect the presence of the DNA of all individuals within the mixture.

In some instances, workflow inefficiencies can be similarly challenging for certain nucleic acid interrogation applications. One non-limiting example of this is in clinical microbiology testing. Sometimes it is desired to rapidly detect the nature of one or more infectious organisms, for example, a microbial or polymicrobial bloodstream infection where some organisms are resistant to particular antibiotics based on a unique genetic variant they carry, but the time it takes to culture and empirically determine antibiotic sensitivity of the infectious organisms is much longer than the time within which a therapeutic decision about antibiotics to be used for treatment must be made. DNA sequencing of DNA from the blood (or other infected tissue or body fluid) has the potential to be more rapid, and DS among other high accuracy sequencing methods, for example, could very accurately detect therapeutically important minority variants in the infectious population based on DNA signature. As workflow turn-around time to data generation can be critical for determining treatment options (e.g., as in the example used herein), applications to increase the speed to arrive at data output would also be desirable.

Disclosed further herein are methods and compositions for targeted nucleic acid sequence enrichment for a variety of nucleic acid material interrogation applications. In particular, some aspects of the present technology are directed to methods and compositions for targeted nucleic acid material enrichment and uses of such enrichment for error-corrected nucleic acid sequencing applications that provide improvement in the cost, conversion of molecules sequenced and the time efficiency of generating labeled molecules for targeted ultra-high accuracy sequencing.

### I. Selected Embodiments of Methods and Reagents for Enrichment of Nucleic Acid Material

In some embodiments, provided methods provide targeted enrichment strategies compatible with the use of molecular barcodes for error correction. Other embodiments provide methods for non-amplification based targeted enrichment strategies compatible with DDS and other sequencing strategies (e.g., single molecule sequencing modalities and interrogations) that do not use molecular barcoding.

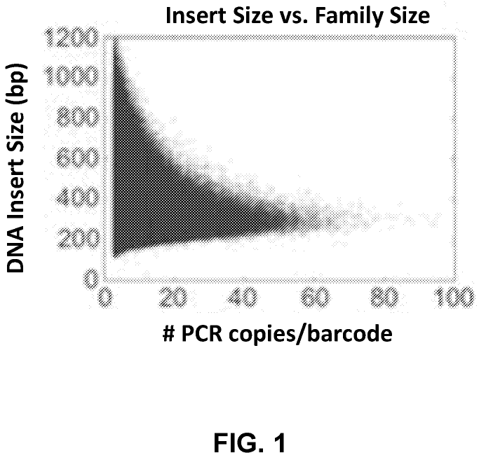

In some embodiments, it is advantageous to process nucleic acid material so as to improve the efficiency, accuracy, and/or speed of a sequencing process. In accordance with further aspects of the present technology, the efficiency of, for example, DS can be enhanced by targeted nucleic acid fragmentation. Classically, nucleic acid (e.g., genome, mitochondrial, plasmid, etc.) fragmentation is achieved either by physical shearing (e.g., sonication) or relatively non-sequence-specific enzymatic approaches that utilize an enzyme cocktail to cleave DNA phosphodiester bonds. The result of either of the above methods is a sample where the intact nucleic acid material (e.g., genomic DNA (gDNA)) is reduced to a mixture of randomly or semi-randomly sized nucleic acid fragments. While effective, these approaches generate variable sized nucleic acid fragments which may result in amplification bias (e.g., short fragments tend to PCR amplify more efficiently than longer fragments and may cluster amplify more easily during polony formation) and uneven depth of sequencing. For example, FIG. 1 is a graph plotting a relationship between nucleic acid insert size and resulting family size following amplification of a population of DNA molecules tagged with diverse molecular barcodes during library preparation. As shown in FIG. 1, because shorter fragments tend to preferentially amplify, on average a greater number of copies of each of these shorter fragments are generated and sequenced, providing a disproportionate level of sequencing depth of these regions.

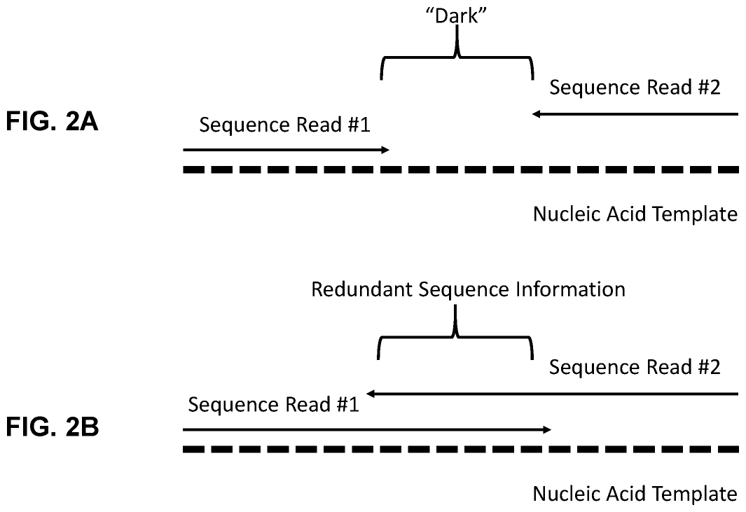

Further, with longer fragments, a portion of DNA between the limit of a sequencing read (or between the ends of paired end sequencing reads) cannot be interrogated if it extends beyond the maximum read length of the sequencing platform and is “dark” despite being successfully ligated, amplified and captured (FIG. 2A). Likewise, with short fragments, and when using paired-end sequencing, overlapped reads in covering the same sequence in the middle of a molecule from both reads provides redundant information and is cost-inefficient (FIG. 2B). Random or semi-random nucleic acid fragmentation may also result in unpredictable break points in target molecules that yield fragments that may not have complementarity or reduced complementarity to a bait strand for hybrid capture, thereby decreasing a target capture efficiency. Random or semi-random fragmentation can also break sequences of interest and or lead to very small or very large fragments that are lost during other stages of library preparation and can decrease data yield and efficiency.

One other problem with many methods of random fragmentation, particularly mechanical or acoustic methods, is that they introduce damage beyond double-stranded breaks that can render portions of double-stranded DNA no longer double-stranded. For example, mechanical shearing can create 3′ or 5′ overhangs at the ends of molecules and single-stranded nicks or gaps in the middle of molecules. These single-stranded portions amenable to adapter ligation, such as a cocktail of “end repair” enzymes, are used to artificially render it double-stranded once again, and which can be a source of artificial errors (such as, e.g., “pseudoduplex molecules” as described herein). In many embodiments, maximizing the amount of double-stranded nucleic acid of interest that remains in native double-stranded form during handling is optimal In addition, the high energies involved with many methods of random or semi-random mechanical fragmentation increase the abundance of DNA damage, such as, oxidation, deamination or other adduct formation that may be mutagenic or inhibitory during amplification or sequencing, and may introduce artefactual base calls or reduced signal. Some random or semi-random enzymatic fragmentation methods can similarly leave mutagenic or blocking “scars” at sites of partial cutting.

Additionally, for DS processing, both strands of an original target nucleic acid molecule must be successfully ligated. For example, in embodiments where adapters are ligated to both a 5′ end and a 3′ end of a molecule, four phosphodiester bonds must be successfully produced. If one of these bonds fails to form, it becomes impossible to amplify and sequence both strands of that molecule. As stated above, failures to form the necessary bonds may occur for multiple reasons including, for example, damage to the ends of the target double-stranded nucleic acid molecules, incomplete end-repair or tailing of the library fragment, incomplete synthesis or damaged adapter molecules, contaminations the ligation or preceding reactions, for example, with undesired enzymatic activities (e.g., exonuclease activity that can disrupt the ligatable ends of the adapters or library fragments, or degradation of the ligation enzymes, rendering their multi-order catalytic activity inefficient), among other causes. Damage to the ends of library fragments is can be particularly common with high-energy ultrasonic or other mechanical DNA fragmentation.

In addition to successful adapter ligation, both first and second strands of the adapter-target nucleic acid complexes must be amplifiable to achieve duplex sequence accuracy. If, for example, a particular strand of a target nucleic acid molecule is nicked or damaged in a way that a polymerase cannot traverse, amplification of the particular strand will not occur, and a Duplex Consensus Sequence read cannot be generated. Non-traversable damage can be introduced, by way of non-limiting examples, by ultrasonic DNA fragmentation, high temperature or prolonged enzymatic steps or single-stranded nicking activity in library preparation.

Accordingly, DS, among other applications, may benefit from efficiency improvements by utilizing one or more methods for enrichment of target nucleic acid within samples, including enrichment of target nucleic acid material prior to amplification steps. Regardless of the underlying method, detection of rare nucleic acid variants requires screening a large number of molecules; however, the more molecules (i.e. genomic equivalents) that are simultaneously prepared into a library, the lower the relative efficiency of the process.

Various aspects of the present technology provide methods, reagents, and nucleic acid libraries and kits for enrichment of nucleic acid material for sequencing applications and other nucleic acid interrogations. Additional aspects of the present technology provide multiple solutions to improve both the conversion efficiency and workflow efficiency of DS and other sequencing modalities, to overcome the majority of limitations enumerated above.

Some aspects of the present technology are directed to methods for enriching region(s) of interest using the Clustered Regularly Interspaced Short Palindromic Repeats (CRISPR) programmable endonuclease system. In other aspects, CRISPER-like or other programmable endonucleases such as zinc-finger nucleases, TALEN nucleases or other sequence-specific endonucleases such as homing endonucleases or simple restriction nucleases or derivatives thereof can be used alone or in combination as part of the disclosed technology.

In particular, CRISPR/Cas9 (or other programmable or non-programmable endonucleases or a combination thereof) can be used to selectively cleave a nucleic backbone in one or more defined or semi-defined region to functionally excise one or more sequence regions of interest from within a longer nucleic acid molecule wherein the excised target region(s) are designed to be of one or more predetermined, or substantially predetermined lengths, thus enabling enrichment of one or more nucleic acid target region of interest via size selection prior to library preparation for sequencing applications such as DS. In other embodiments, CRISPR/Cas9 (or other programmable endonuclease or non-programmable endonuclease or a combination thereof) can be used to selectively excise one or more sequence regions of interest wherein the excised target region(s) are designed to have a substantially predetermined length and sequence of an overhang, These programmable endonucleases can be used either alone or in combination with other forms of targeted nucleases, such as restriction endonuclease, or other enzymatic or non-enzymatic methods for cleaving nucleic acids.

In some embodiments, a provided method may include the steps of providing a nucleic acid material, cutting the nucleic acid material with a targeted endonuclease (e.g., a ribonucleoprotein complex) so that a target region or regions of a substantially predetermined length is separated or enriched from the rest of the nucleic acid material, and analyzing the cut target region. In other embodiments the cut region or regions can be negatively enriched (i.e depleted) from the rest of the nucleic acid material and and not analyzed. In some embodiments, provided methods may further include ligating at least one SMI and/or adapter sequence to at least one of the 5′ or 3′ ends of the cut target region of predetermined length. In some embodiments, analyzing may be or comprise quantitation and/or sequencing.

In some embodiments, quantitation may be or comprise spectrophotometric analysis, real-time PCR, and/or fluorescence-based quantitation (e.g., using fluorescent dye tagging). In some embodiments, sequencing may be or comprise Sanger sequencing, shotgun sequencing, bridge PCR, nanopore sequencing, single molecule real-time sequencing, ion torrent sequencing, pyrosequencing, digital sequencing (e.g., digital barcode-based sequencing), sequencing by ligation, polony-based sequencing, electrical current-based sequencing (e.g., tunneling currents), sequencing via mass spectroscopy, microfluidics-based sequencing, Illumina Sequencing, next generation sequencing, massively parallel and any combination thereof.

In some embodiments, a targeted endonuclease is or comprises at least one of a CRISPR-associated (Cas) enzyme (e.g., Cas9 or Cpf1) or other ribonucleoprotein complex, a homing endonuclease, a zinc-fingered nuclease, a transcription activator-like effector nuclease (TALEN), an argonaute nuclease, a megaTAL nuclease, a meganuclease, and/or a restriction endonuclease. In some embodiments, more than one targeted endonuclease may be used (e.g., 2, 3, 4, 5, 6, 7, 8, 9, 10 or more). In some embodiments, a targeted nuclease may be used to cut at more than one potential target region of predetermined length (e.g., 2, 3, 4, 5, 6, 7, 8, 9, 10 or more). In some embodiments where there is more than one target region of predetermined length, each target region may be of the same (or substantially the same) length. In some embodiments where there is more than one target region of predetermined length at least two of the target regions of predetermined length differ in length (e.g., a first target region with a length of 100 bp and a second target region with a length of 1,000 bp).

The present disclosure, among other things, provides methods and reagents for affinity-based enrichment of target nucleic acid material. In some embodiments including such methods, one or more capture labels or moieties may be used for enrichment/selection of desired target nucleic acid material from samples comprising genomic material, off-target nucleic acid material, contaminating nucleic acid material, nucleic acid material from mixed samples, cfDNA material, etc. For example, some embodiments comprise use of one or more capture labels/moieties for positive enrichment/selection of desired target nucleic acid material (e.g., fragments comprising target sequence or genomic regions of interest, targeted genomic regions of interest within unfragmented genomic DNA). In other embodiments, capture labels may be use for negative enrichment/selection to exclude or reduce the abundance of non-desired genomic material.

For example, in some embodiments including positive enrichment, an adapter oligonucleotide can have a capture label that is or comprises an affixed chemical moiety (e.g. biotin) that may be used to isolate or separate desired adapter-nucleic acid complexes via capture in one or more subsequent purification steps, for example, via an extraction moiety (e.g. streptavidin) bound to a functionalized surface (e.g. a paramagnetic bead or other form of bead). In some embodiments including negative enrichment, a capture label that is or comprises an affixed chemical moiety (e.g. biotin) may be used to purify out or separate undesired genomic material ligated or attached to an adapter (or other probe comprising the capture label) (e.g., off-target nucleic acid fragments, etc.) via capture in one or more subsequent purification steps, for example, via an extraction moiety (e.g. streptavidin) bound to a functionalized surface (e.g. a paramagnetic bead or other form of bead)

Size-Based Enrichment of Nucleic Acid Material

In some embodiments, provided methods and compositions take advantage of a targeted endonuclease (e.g., a ribonucleoprotein complex (CRISPR-associated endonuclease such as Cas9, Cpf1), a homing endonuclease, a zinc-fingered nuclease, a TALEN, an argonaute nuclease, a meganuclease, a restriction endonuclease and/or a meganuclease (e.g., megaTAL nuclease, etc.), or a combination thereof) or other technology capable of cutting a nucleic acid material (e.g., one or more restriction enzymes) to excise a target sequence of interest in an optimal fragment size for sequencing. In some embodiments, targeted endonucleases have the ability to specifically and selectively excise precise sequence regions of interest. By pre-selecting cut sites, for example with a programmable endonuclease (e.g., CRISPR-associated (Cas) enzyme/guideRNA complex) that result in fragments of predetermined and substantially uniform sizes, the biases and the presence of uninformative reads can be drastically reduced. Furthermore, because of the size differences between the excised fragments and the remaining non-cut DNA, a size selection step (as further described below) can be performed to remove the large off-target regions, thus pre-enriching the sample prior to any further processing steps. The need for end-repair steps may be reduced or eliminated as well, thus saving time and risk of pseudoduplex challenges and, in some cases, reducing or eliminating the need for computational trimming of data near the end of molecules, thus improving efficiency. An additional advantage of thus targeted enzymatic fragmentation is the potential to reduce nicks or nucleic acid adducts or other forms of damage caused by mechanical fragmentation methods.

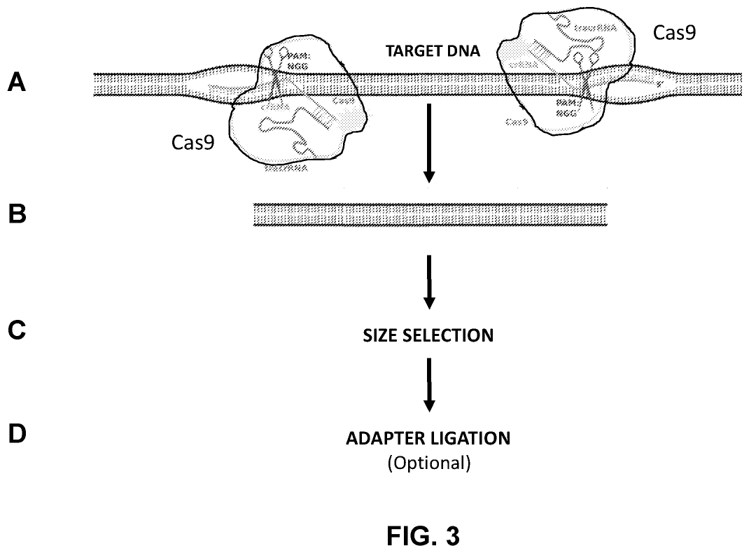

A method termed CRISPR-DS, allows for very high on-target enrichment (which may reduce need for subsequent hybrid capture steps), which can significantly decrease time and cost as well as increase conversion efficiency. FIG. 3 is a schematic illustrating steps of a method for generating targeted fragment sizing with CRISPR/Cas9 in accordance with various embodiments of the present technology. For example, CRISPR/Cas9 can be used to cut at one or more specific sites (e.g., a protospacer adjacent motif or “PAM” site) within a target sequence (FIG. 3, Panel A) by way of gRNA-facilitated binding of Cas9. Cas9 directed cleavage releases a blunt-ended double-stranded target DNA fragment of known length as shown in Panel B. FIG. 3, Panel C depicts a further processing step for positive enrichment/selection of the target DNA fragments via size selection. One method of isolating the excised target portion includes using SPRI/Ampure bead and magnet purification to remove high molecular weight DNA while leaving the pre-determined shorter fragment. In other embodiments, the excised portion of pre-determined length can be separated from non-desirable DNA fragments and other high molecular weight genomic DNA (if applicable) using a variety size selection methods including, but not limited to gel electrophoresis, gel purification, liquid chromatography, size exclusion purification, and/or filtration purification methods, among others. Following size selection, CRISPR-DS methods may include steps consistent with DS method steps including A-tailing (CRISPR/Cas9 excision leaves blunt ends), ligation of adapters (e.g., DS adapters), duplex amplification, an optional capture step and amplification (e.g., PCR) before sequencing of each strand and generating a duplex consensus sequence. In addition to improvement in workflow efficiencies, CRISPR-based size selection/target enrichment provides optimal fragment lengths for high efficiency amplification and sequencing steps. Aspects of CRISPR-DS are disclosed in International Patent Publication No. WO/2018/175997, which is incorporated herein by reference in its entirety.

In certain embodiments, CRISPR-DS solves multiple common problems associated with NGS, including, e.g. inefficient target enrichment, which may be optimized by CRISPR-based size selection; sequencing errors, which can be removed using DS methodology for generating an error-corrected duplex consensus sequence; and uneven fragment size, which is mitigated by predesigned CRISPR/Cas9 fragmentation. As will be appreciated by one of skill in the art, as described herein, CRISPR-DS may have application for sensitive identification of mutations in situations in which samples are DNA-limited, such as forensics and early cancer detection applications, among others.

The in vitro digestion of DNA material with Cas9 Nuclease makes use of the formation of a ribonucleoprotein complex, which both recognizes and cleaves a pre-determined site (e.g., a PAM site, FIG. 3, Panel A). This complex is formed with guide RNAs (“gRNAs”, e.g., crRNA+tracrRNA) and Cas9. For multiplex cutting, the gRNAs can be complexed by pooling all the crRNAs, then complexing with tracrRNA, or by complexing each crRNA and tracrRNA separately, then pooling. In some embodiments, the second option may be preferred because it eliminates competition between crRNAs. Other CRISPER systems using different Cas proteins may rely on different PAM motif sequences, or not require PAM motif sequences or rely on other forms of nucleic-acid sequences to guide delivery of the nuclease to the targeted nucleic acid region.

In some embodiments, the nucleic acid material comprises nucleic acid molecules of a substantially uniform length. In some embodiments, a substantially uniform length is between about 1 and 1,000,000 bases). For example, in some embodiments, a substantially uniform length may be at least 1; 2; 3; 4; 5; 6; 7; 8; 9; 10; 15; 20; 25; 30; 35; 40; 50; 60; 70; 80; 90; 100; 120; 150; 200; 300; 400; 500; 600; 700; 800; 900; 1000; 1200; 1500; 2000; 3000; 4000; 5000; 6000; 7000; 8000; 9000; 10,000; 15,000; 20,000; 30,000; 40,000; or 50,000 bases in length. In some embodiments, a substantially uniform length may be at most 60,000; 70,000; 80,000; 90,000; 100,000; 120,000; 150,000; 200,000; 300,000; 400,000; 500,000; 600,000; 700,000; 800,000; 900,000; or 1,000,000 bases. By way of specific, non-limiting example, in some embodiments, a substantially uniform length is between about 100 to about 500 bases. In some embodiments a size selection step, such as those described herein, may be performed before any particular amplification step. In some embodiments a size selection step, such as those described herein, may be performed after any particular amplification step. In some embodiments, a size selection step such as those described herein may be followed by an additional step such as a digestion step and/or another size selection step. In some embodiments size selection may occur before or after a step of ligation of adapters. In some embodiments size selection may occur concurrently to a cutting steps. In some embodiments size selection may occur after a cutting step.

In addition to use of targeted endonuclease(s), any other application appropriate method(s) of achieving nucleic acid molecules of a substantially uniform length may be used. By way of non-limiting example, such methods may be or include use of one or more of: an agarose or other gel, gel electrophoresis, an affinity column, HPLC, PAGE, filtration, gel filtration, exchange chromatography, SPRI/Ampure type beads, or any other appropriate method as will be recognized by one of skill in the art.

In some embodiments, processing a nucleic acid material so as to produce nucleic acid molecules of substantially uniform length (or mass), may be used to recover one or more desired target region from a sample (e.g., a target sequence of interest). In some embodiments, processing a nucleic acid material so as to produce nucleic acid molecules of substantially uniform length (or mass), may be used to exclude specific portions of a sample (e.g., nucleic acid material from a non-desired species or non-desired subject of the same species). In some embodiments, nucleic acid material may be present in a variety of sizes (e.g., not as substantially uniform lengths or masses).

In some embodiments, more than one targeted endonuclease or other method for providing nucleic acid molecules of a substantially uniform length may be used (e.g., 2, 3, 4, 5, 6, 7, 8, 9, 10 or more). In some embodiments, a targeted nuclease may be used to cut at more than one potential target region of a nucleic acid material (e.g., 2, 3, 4, 5, 6, 7, 8, 9, 10 or more). In some embodiments where there is more than one target region of a nucleic acid material, each target region may be of the same (or substantially the same) length. In some embodiments where there is more than one target region of a nucleic acid material, at least two of the target regions of known length differ in length (e.g., a first target region with a length of 100 bp and a second target region with a length of 1,000 bp).

In some embodiments, multiple targeted endonucleases (e.g., programmable endonucleases) may be used in combination to fragment multiple regions of the target nucleic acid of interest. In some embodiments, one or more programmable targeted endonucleases may be used in combination with other targeted nucleases. In some embodiments one or more targeted endonucleases may be used in combination with random or semi-random nucleases. In some embodiments, one or more targeted endonucleases may be used in combination with other random or semi-random methods of nucleic acid fragmentation such as mechanical or acoustic shearing. In some embodiments, it may be advantageous to perform cleavage in sequential steps with one or more intervening size selection steps. In some embodiments where targeted fragmentation is used in combination with random or semi-random fragmentation, the random or semi-random nature of the latter may be useful for serving the purpose of a unique molecular identifier (UMI) sequence. In some embodiments where targeted fragmentation is used in combination with random or semi-random fragmentation, the random or semi-random nature of the latter may be useful for facilitating sequencing of regions of a nucleic acid that are not easily cleaved in a targeted way such as long or highly repetitive regions or regions with substantial similarities to other regions in a genome or genomes that may be otherwise challenging to enrich by traditional methods of hybrid capture.

Targeted Endonucleases

Targeted endonucleases (e.g., a CRISPR-associated ribonucleoprotein complex, such as Cas9 or Cpf1, a homing nuclease, a zinc-fingered nuclease, a TALEN, a megaTAL nuclease, an argonaute nuclease, and/or derivatives thereof) can be used to selectively cut and excise targeted portions of nucleic acid material for purposes of enriching such targeted portions for sequencing applications. In some embodiments, a targeted endonuclease can be modified, such as having an amino acid substitution for provided, for example, enhanced thermostability, salt tolerance and/or pH tolerance or enhanced specificity or alternate PAM site recognition or higher affinity for binding. In other embodiments, a targeted endonuclease may be biotinylated, fused with streptavidin and/or incorporate other affinity-based (e.g., bait/prey) technology. In certain embodiments, a targeted endonuclease may have an altered recognition site specificity (e.g., SpCas9 variant having altered PAM site specificity). In other embodiments, a targeted endonuclease may be catalytically inactive so that cleavage does not occur once bound to targeted portions of nucleic acid material. In some embodiments, a targeted endonuclease is modified to cleave a single strand of a targeted portion of nucleic acid material (e.g., a nickase variant) thereby generating a nick in the nucleic acid material. CRISPR-based targeted endonucleases are further discussed herein to provide a further detailed non-limiting example of use of a targeted endonuclease. We note that the nomenclature around such targeted nucleases remains in flux. For purposes herein, we use the term “CRISPER-based” to generally mean endonucleases comprising a nucleic acid sequence, the sequence of which can be modified to redefine a nucleic acid sequence to be cleaved. Cas9 and CPF1 are examples of such targeted endonucleases currently in use, but many more appear to exist different places in the natural world and the availability of different varieties of such targeted and easily tunable nucleases is expected to grow rapidly in the coming years. For example, Cas12a, Cas13, CasX and others are contemplated for use in various embodiments. Similarly, multiple engineered variants of these enzymes to enhance or modify their properties are becoming available. Herein, we explicitly contemplate use of substantially functionally similar targeted endonucleases not explicitly described herein or not yet discovered, to achieve a similar purpose to disclosures described within.

Restriction Endonucleases

It is specifically contemplated that any of a variety of restriction endonucleases (i.e., enzymes) may be used to provide nucleic acid material of substantially uniform length and/or to excise targeted regions of nucleic acid material. Generally, restriction enzymes are typically produced by certain bacteria/other prokaryotes and cleave at, near or between particular sequences in a given segment of DNA.

It will be apparent to one of skill in the art that a restriction enzyme is chosen to cut at a particular site or, alternatively, at a site that is generated in order to create a restriction site for cutting. In some embodiments, a restriction enzyme is a synthetic enzyme. In some embodiments, a restriction enzyme is not a synthetic enzyme. In some embodiments, a restriction enzyme as used herein has been modified to introduce one or more changes within the genome of the enzyme itself. In some embodiments, restriction enzymes produce double-stranded cuts between defined sequences within a given portion of DNA.

While any restriction enzyme may be used in accordance with some embodiments (e.g., type I, type II, type III, and/or type IV), the following represents a non-limiting list of restriction enzymes that may be used: AluI, ApoI, AspHI, BamHI, BfaI, BsaI, CfrI, DdeI, DpnI, DraI, EcoRI, EcoRII, EcoRV, HaeII, HaeIII, HgaI, HindII, HindIII, HinFI, HPYCH4III, KpnI, MamI, MNL1, MseI, MstI, MstII, NcoI, NdeI, NotI, Pad, PstI, PvuI, PvuII, RcaI, RsaI, SacI, SacII, SaII, Sau3AI, ScaI, SmaI, SpeI, SphI, StuI, TaqI, XbaI, XhoI, XhoII, XmaI, XmaII, and any combination thereof. An extensive, but non-exhaustive list of suitable restriction enzymes can be found in publically-available catalogues and on the internet (e.g., available at New England Biolabs, Ipswich, Mass., U.S.A.). It is understood by one experienced in the art that a variety of enzymes, ribozymes or other nucleac acid modifying enzymes that can, alone or in combination, be used to target phosphodiester backbone cleavage of a nucleic acid molecule that can achieve the same purpose may not be included or yet discovered on the above list. A variety of nucleic acid modifying enzymes can recognize base modifications (e.g. CpG methylation) which can be used to target further modification of the adjacent nucleic acid sequence (e.g. to generate an abasic site) that can be cleaved (e.g. by an enzyme with lyase activity). As such, substantial sequence specificity of cleavage can be achieved based on recognition of DNA or RNA modifications and this can be used alone or in combination with targeted endonucleases to achieve targeted nucleic acid fragmentation.

Methods for Negative and Positive Enrichment/Selection of Nucleic Acid Material

In some embodiments, provided methods and compositions take advantage of a targeted endonuclease (e.g., a ribonucleoprotein complex (CRISPR-associated endonuclease such as Cas9, Cpf1), a homing endonuclease, a zinc-fingered nuclease, a TALEN, an argonaute nuclease, and/or a meganuclease (e.g., megaTAL nuclease, etc.), or a combination thereof) or other technology capable of site-directed interaction with nucleic acid material, to positively enrich for desired (on-target) nucleic acid molecules. Other embodiments provide methods and such compositions to negatively enrich/select for desired nucleic acid molecules by way of removing undesired (e.g., off-target) nucleic acid material from the sample. Some embodiments described herein combine both positive and negative enrichment schemes. In some embodiments, provided methods may further include ligating at least one SMI and/or adapter sequence to at least one of the 5′ or 3′ ends of enriched target regions. In some embodiments, analyzing may be or comprise quantitation and/or sequencing.

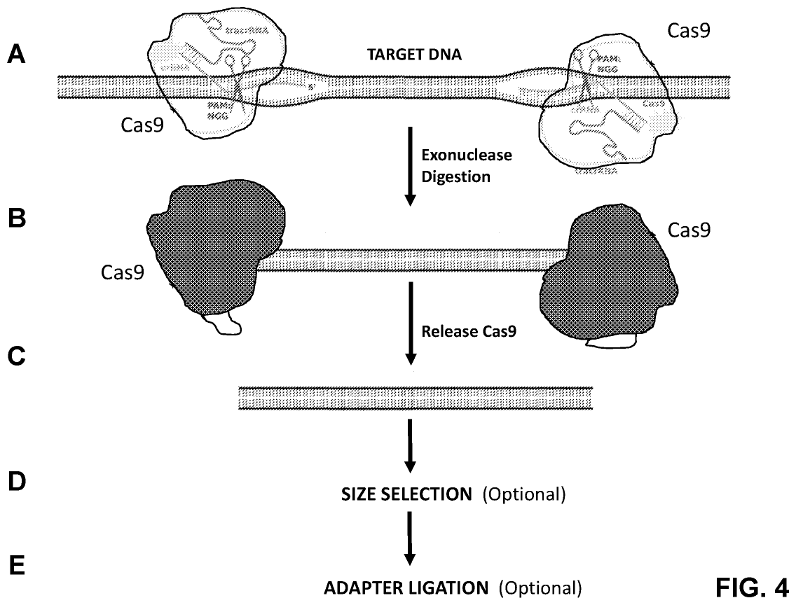

In some embodiments, negative enrichment/selection of target nucleic acid material can be facilitated by removal or destruction of non-target or undesired nucleic acid material. FIG. 4 is a schematic illustrating steps of a method for generating targeted nucleic acid fragment with a substantially known/selected length with a CRISPR/Cas9 variant in accordance with an embodiment of the present technology. Using a CRISPR/Cas9 ribonucleoprotein complex, optionally one having enhanced thermostability and/or engineered to remain bound to dsDNA in suitable conditions (e.g., until removed, enzyme displacement, etc.), Panel A illustrates gRNA-facilitated binding of the variant Cas9 to targeted DNA sites as described above. In one embodiment, and following cleavage and while Cas9 remains bound to the cleaved 5′ and 3 ends of the target DNA fragment, the sample can be treated with an exonuclease to hydrolyze exposed phosphodiester bonds at exposed 3′ or 5′ ends of DNA (Panel B). During exonuclease treatment, undesired or non-targeted DNA will be destroyed through the enzymatic activity leaving only the exonuclease-resistant target dsDNA fragment. As shown in FIG. 4, the bound ribonucleoprotein complexes can provide exonuclease protection. Following negative enrichment/selection of the target DNA fragment via exonuclease destruction of non-targeted DNA, Cas9 is disassociated from the DNA and releases a blunt-ended double-stranded target DNA fragment of known length as shown in Panel C. In some embodiments, the method may also include steps incorporating positive enrichment/selection schemes such using size selection (Panel D). In some embodiments, enriching for fragments of desired and/predicted target size can further filter out genomic fragments that remain undigested and/or were protected by off-target Cas9 binding Optionally, as depicted in Panel E, the enriched DNA fragments can be ligated to adapters for nucleic acid interrogation, such sequencing. For example, the blunt ends of the target fragment can be directly ligated to blunt-ended adapters. Aspects of ligating adapters to the cleaved double-stranded nucleic acid material can include end-repair and 3′-dA-tailing of the fragments, if required in a particular application. In other embodiments, further processing of the fragments to generate suitable ligateable ends of the fragment can include can be any of a variety of forms or steps to form a ligatable end having, for example, a blunt end, an A-3′ overhang, a “sticky” end comprising a one nucleotide 3′ overhang, a two nucleotide 3′ overhang, a three nucleotide 3′ overhang, a 4, 5, 6, 7, 8, 9, 10, 11, 12, 13, 14, 15, 16, 17, 18, 19, 20 or more nucleotide 3′ overhang, a one nucleotide 5′ overhang, a two nucleotide 5′ overhang, a three nucleotide 5′ overhang, a 4, 5, 6, 7, 8, 9, 10, 11, 12, 13, 14, 15, 16, 17, 18, 19, 20 or more nucleotide 5′ overhang, among others. The 5′ base of the ligation site can be phosphorylated and the 3′ base can have a hydroxyl group, or either can be, alone or in combination, dephosphorylated or dehydrated or further chemically modified to either facilitate enhanced ligation of one strand to prevent ligation of one strand, optionally, until a later time point.

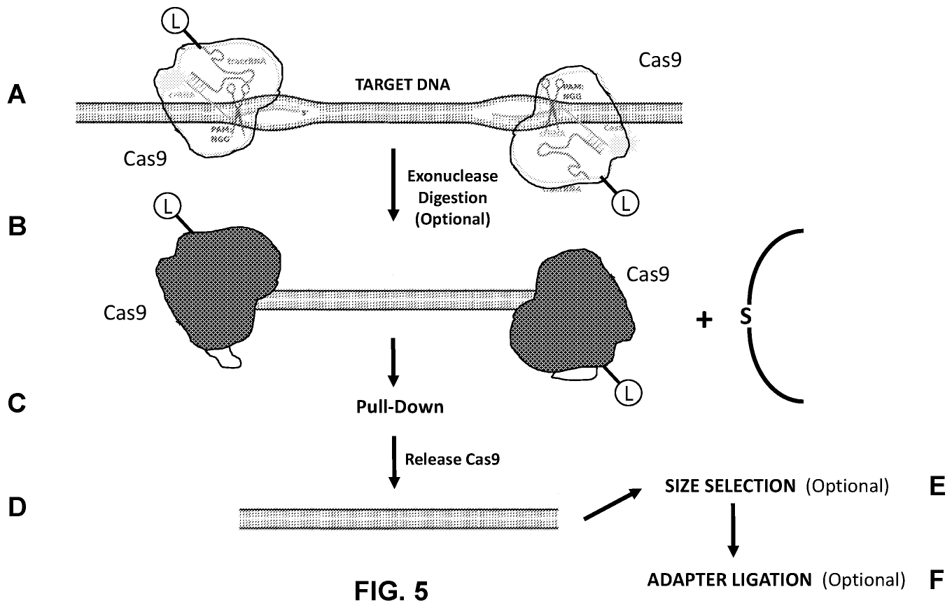

In another embodiment, positive enrichment/selection of target nucleic acid material using CRISPR/Cas can be facilitated by affinity-based enrichment of target nucleic acid material. FIG. 5 is a schematic illustrating steps of a method for generating targeted nucleic acid fragment with a substantially known/selected length with a CRISPR/Cas9 variant in accordance with another embodiment of the present technology. Panel A illustrates using a CRISPR/Cas9 ribonucleoprotein complex, which has optionally be further engineered to remain strongly bound to DNA in suitable condition (as described above), wherein the ribonucleoprotein complex comprises a capture label (e.g., biotin). The capture label can be incorporated on the gRNA (e.g., crRNA, tracrRNA) or on the Cas9 protein. Accordingly, the ribonucleoprotein complex provides an affinity label for later pull-down steps.

Guide RNA (gRNA)-facilitated binding of the variant Cas9 ribonucleoprotein complex presenting the capture label is followed by cleavage of the double-stranded target DNA. Following cleavage and while Cas9 remains bound to the cleaved 5′ and 3 ends of the target DNA fragment, the reaction mixture is brought into contact with a functionalized surface with one or more extraction moieties bound thereto. The provided extraction moieties are capable of binding to the capture label (e.g. a streptavidin bead where the capture label is biotin) for immobilization and separation of molecules bearing the capture label. In particular, the extraction moiety can be any member of a binding pair, such as biotin/streptavidin or hapten/antibody or complementary nucleic acid sequences (DNA/DNA pair, DNA/RNA pair, RNA/RNA pair, LNA/DNA pair, etc.). In the illustrated embodiment, a capture label that is attached to a CRISPR/Cas9 ribonucleoprotein complex that is bound to a (cleaved) target dsDNA fragment is captured by its binding pair (e.g., the extraction moiety) which is attached to an isolatable moiety (e.g., such as a magnetically attractable particle or a large particle that can be sedimented through centrifugation). Accordingly, the capture label can be any type of molecule/moiety that allows affinity separation of nucleic acids associated with (e.g., bound by Cas9) the capture label from nucleic acids lacking association with the capture label. An example of a capture label is biotin which allows affinity separation by binding to streptavidin linked or linkable to a solid phase or an oligonucleotide, which in turn allows affinity separation through binding to a complementary oligonucleotide linked or linkable to a solid phase. Undesired or non-targeted nucleic acid material can remain free in solution. Beneficially, free/unbound nucleic acid material, which does not bear or is associated with any capture label, can be effectively removed/separated from the desired target nucleic acid material. In further embodiments, the functionalized surface (S) maybe washed to remove residual byproducts or other contaminants.

Using the affinity-based enrichment scheme illustrated in FIG. 5, undesired or non-targeted nucleic acid material can be substantially reduced in abundance. Collection of the desired/target nucleic acid fragments may be accomplished in any application-appropriate manner. By way of specific example, in some embodiments, collection of desired nucleic acid material may be accomplished via one or more of removal of the functionalized surface via size filtration, magnetic methods, electrical charge methods, centrifugation density methods or any other methods or, collection of elution fractions if using column-based purification methods or similar, or by any other commonly understood purification practice by one experienced in the art.

In some embodiments, the affinity-based positive enrichment steps can be combined or used in conⱼunction with negative enrichment steps. For example, following cleavage and while Cas9 remains bound to the cleaved 5′ and 3 ends of the target DNA fragment (either before or after the affinity-based enrichment step), the sample can be treated with an exonuclease to destroy any unwanted nucleic acid material or contaminants in the sample. After the affinity-based enrichment step and optional negative exonuclease clean up steps depicted in Panels A and B, Cas9 is disassociated from the DNA to release a blunt-ended double-stranded target DNA fragment of known length (Panel D). Optionally, the above enrichment steps can be combined with a size-based enrichment step as described above (Panel E), and in some embodiments, the enriched DNA fragments can be ligated to adapters for nucleic acid interrogation, such sequencing (Panel F) as discussed above.

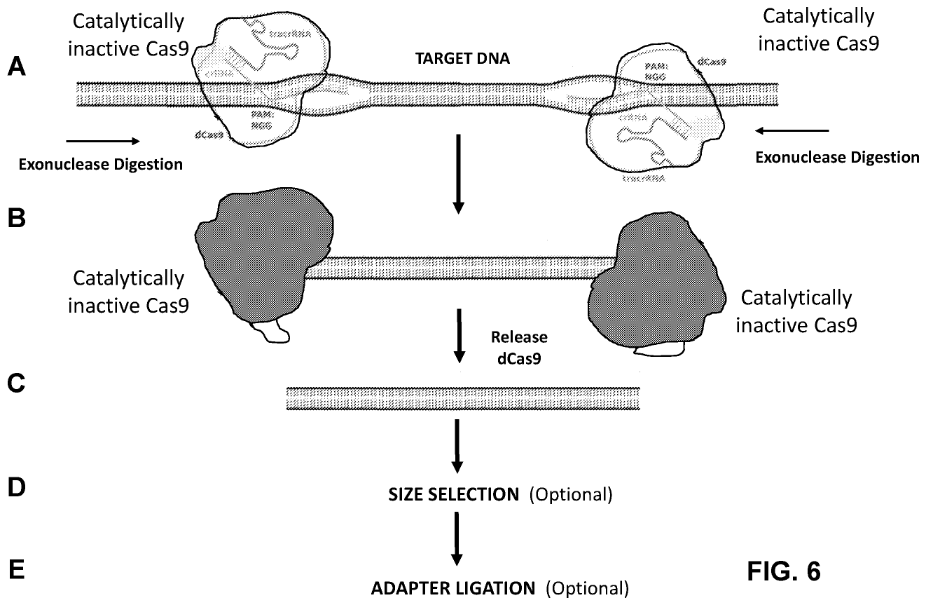

FIG. 6 is a schematic illustrating steps of a method for negative enrichment/selection of target nucleic acid material in accordance with another embodiment of the present technology. For example, enrichment of target double-stranded nucleic acid material can be facilitated by removal or destruction of non-target or undesired nucleic acid material. FIG. 6 illustrates an embodiment of enrichment employing a catalytically inactive variant of Cas9 to generate targeted nucleic acid fragments with a substantially known/selected length. Using a catalytically inactive Cas9 ribonucleoprotein complex engineered to target and selectively bind double-stranded DNA, gRNA-facilitates binding of a pair of catalytically inactive Cas9 variants to flank targeted DNA regions (Panel A). Following binding, the sample can be treated with or more exonucleases to hydrolyze exposed phosphodiester bonds at exposed 3′ or 5′ ends of DNA. The catalytically inactive variant of Cas9 does not cut the target DNA but provides exonuclease resistance such that exonuclease activity cleaves each nucleotide base until blocked by the bound Cas9 complex. Accordingly, exonuclease treatment destroys all non-targeted nucleic acid material in the sample with exposed ends leaving fragments protected by pairs of catalytically inactive Cas9. In certain embodiments, a cocktail of endonucleases and exonucleases can be used to destroy undesired nucleic acid material. For example, endonucleases (e.g., site specific restriction enzymes) can be used to generate multiple exposed 5′ and 3′ ends to allow for exonuclease enzymatic active.

Following negative/enrichment selection of the target DNA fragment via exonuclease destruction of all non-targeted DNA (Panel B), catalytically inactive Cas9 is disassociated from the DNA thereby releasing a double-stranded target DNA fragment of known length as shown in Panel C. As discussed above, additional size selection steps can be implemented for further enrichment of target double-stranded DNA fragments (Panel D) Optionally, the enriched DNA fragments can be polished, blunted, or tailed to form suitable ligatable ends and subsequently ligated to adapters for nucleic acid interrogation, such sequencing (Panel E).

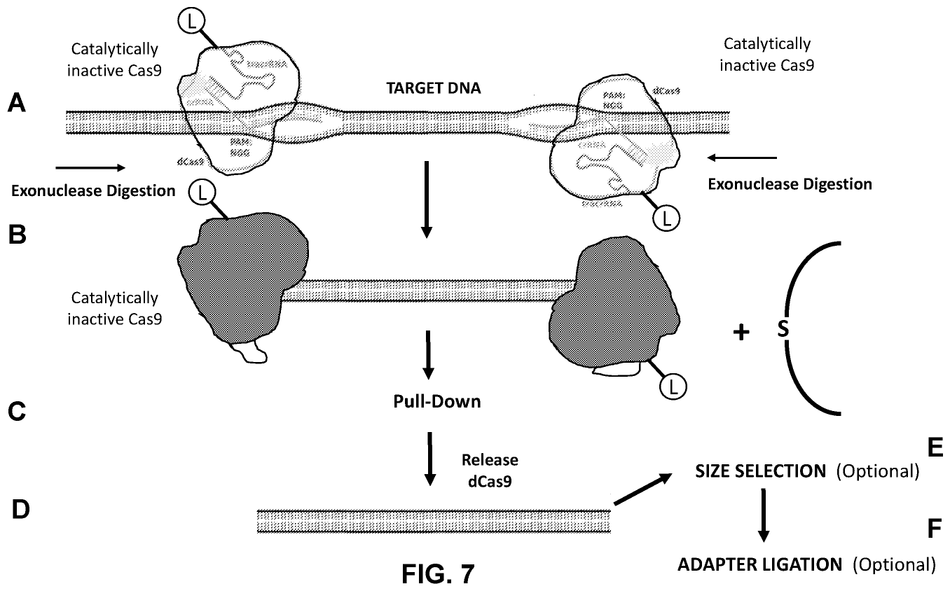

In another embodiment depicted in FIG. 7, both negative and positive enrichment schemes can be implemented using the catalytically inactive variant of Cas9. Panel A illustrates using a catalytically inactive variant of Cas9 in a ribonucleoprotein complex engineered to remain bound to DNA in suitable condition, and wherein the ribonucleoprotein complex comprises a capture label (e.g., on the guide RNA or tethered to the Cas9 protein, for example). Guide RNA (gRNA)-facilitated binding of the catalytically inactive variant Cas9 ribonucleoprotein complex with capture label is followed by addition of an exonuclease to the sample to hydrolyze exposed phosphodiester bonds at exposed 3′ or 5′ ends of DNA. The catalytically inactive variant of Cas9 does not cut the target DNA but provides exonuclease resistance such that exonuclease activity cleaves each nucleotide base until blocked by the bound Cas9 complex. Following negative/enrichment selection of the target DNA fragment via exonuclease destruction of all non-targeted DNA, and while catalytically inactive Cas9 remains bound, step-wise addition of functionalized surfaces (e.g., functionalized surface with one or more extraction moieties bound thereto) that are capable of binding the capture label associated with the ribonucleoprotein complex as it remains bound to the target nucleic acid, can immobilize and/or separate the molecules bearing and/or associated with the capture label from undesired nucleic acid material remaining in the sample (Panel B). In some embodiments, provided methods allow for removal of all or substantially all undesired nucleic acid material in a sample or substantially reduce their abundance. Collection of the desired target nucleic acid material may be accomplished in any application-appropriate manner By way of specific example, in some embodiments, collection of desired target nucleic acid fragments may be accomplished via one or more of removal of the functionalized surface via size filtration, magnetic methods, electrical charge methods, centrifugation density methods or any other methods or, collection of elution fractions if using column-based purification methods or similar, or by any other commonly understood purification practice.

After the affinity-based enrichment step, and as depicted in Panel D, Cas9 is disassociated from the DNA and releases a double-stranded target DNA fragment of known length. Panel E depicts an optional further processing step for positive enrichment/selection of the target DNA fragments via size selection. Optionally, as depicted in Panel F, the enriched DNA fragments can be ligated to adapters for nucleic acid interrogation, such sequencing.

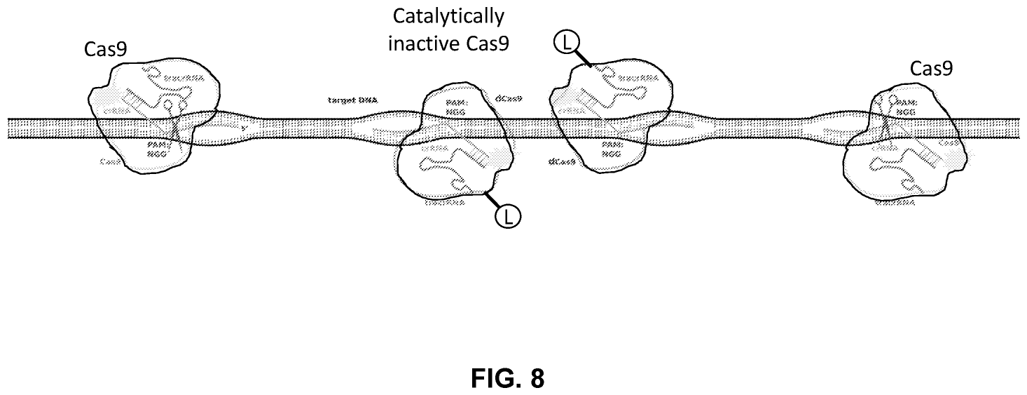

In some embodiments, combinations of catalytically active and catalytically inactive CRISPR/Cas complexes can be used to positively enrich for fragments comprising target double-stranded nucleic acid regions. Referring to FIG. 8, both catalytically active and catalytically inactive Cas9 ribonucleoprotein complexes can be targeted in a sequence-dependent manner to a desired nucleic acid region (e.g., a particular genomic loci) in a sample. Catalytically active Cas 9 ribonucleoprotein complexes are directed to regions flanking a target DNA region and are used to cleave target double-stranded DNA to release a blunt-ended double-stranded target DNA fragment of known length. One or more catalytically inactive ribonucleoprotein complexes bearing a capture label (e.g., biotin) are directed to target sequence regions between the two site selected cleavage sites. Following cleavage of target DNA to release the DNA fragment, addition of functionalized surfaces that are capable of binding a capture label associated with the catalytically inactive ribonucleoprotein complex can facilitate positive enrichment/selection of the target fragment. It will be recognized that many other forms of targeted nucleic acid fragmentation, such as those described above, could substitute for the active Cas9 ribonucleoprotein complexes in this example.

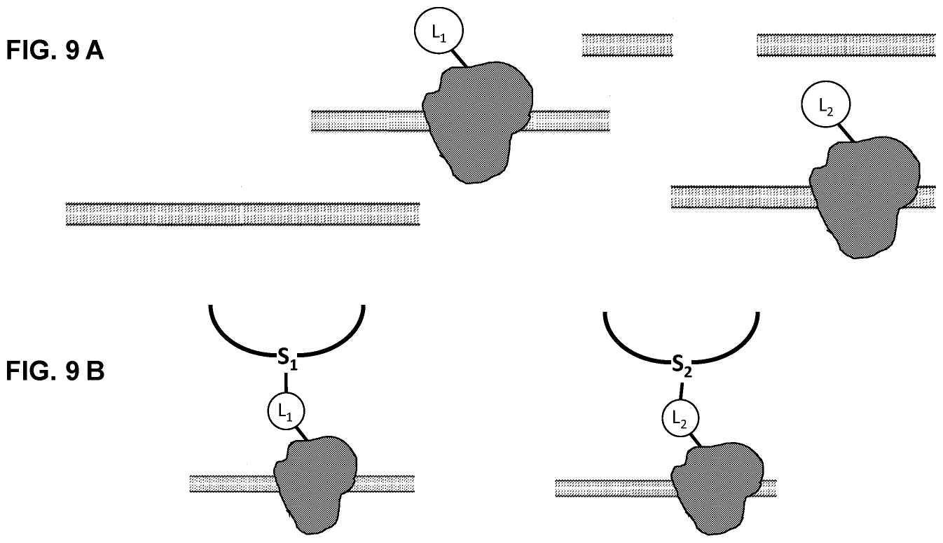

In some embodiments, positive enrichment/selection steps can be taken to enrich for target sequences from sample wherein the nucleic acid material is already fragmented (e.g., mechanically sheared or from a cell free DNA sample (e.g., from a liquid biopsy)). FIGS. 9A and 9B are conceptual illustrations of methods steps for positive enrichment/selection of target nucleic acid fragments using a catalytically inactive variant of Cas 9 ribonucleoprotein complex bearing a capture label as described above. Fragmented double-stranded DNA fragments in a sample (e.g., mechanically sheared, acoustically fragmented, cell free DNA, etc.) can be positively enriched/selected via target directed binding by one or more catalytically inactive Cas9 ribonucleoprotein complex in solution (FIG. 9A).

In some embodiments, a method may include the use of two or more capture labels (e.g., 2, 3, 4, 5, 6, 7, 8, 9, 10 or more) that can be used to differentially tag a plurality of Cas9 ribonucleoprotein complexes. For example, a sample can be enriched for multiple target nucleic acid samples concurrently. While in some embodiments it is contemplated that all Cas9 complexes bear the same capture label (e.g., biotin), such that all targeted sequences can be pulled-down (affinity purified) together in a single sample, in other embodiments, separation of different targeted sequences can be facilitated by incorporating substantially unique capture labels with Cas9 complexes that are directed to target different regions. In some embodiments, at least two capture labels used in a method are different from one another (e.g., a small molecule and a peptide). In some embodiments, inclusion of two or more different capture labels allows for the use of both positive enrichment/selection as well as negative enrichment/selection. Inclusion of two or more capture labels can be helpful, inter alia, in cases where there is a desire to physically separate nucleic acid fragments that comprise different target sequences for later nucleic acid interrogation, e.g., sequencing.

The reaction mixture is brought into contact with a functionalized surface(s) with one or more extraction moieties bound thereto. The provided extraction moieties are capable of binding to the capture label (e.g. a streptavidin bead where the capture label is biotin) for immobilization and separation of molecules bearing the capture label (FIG. 9B).

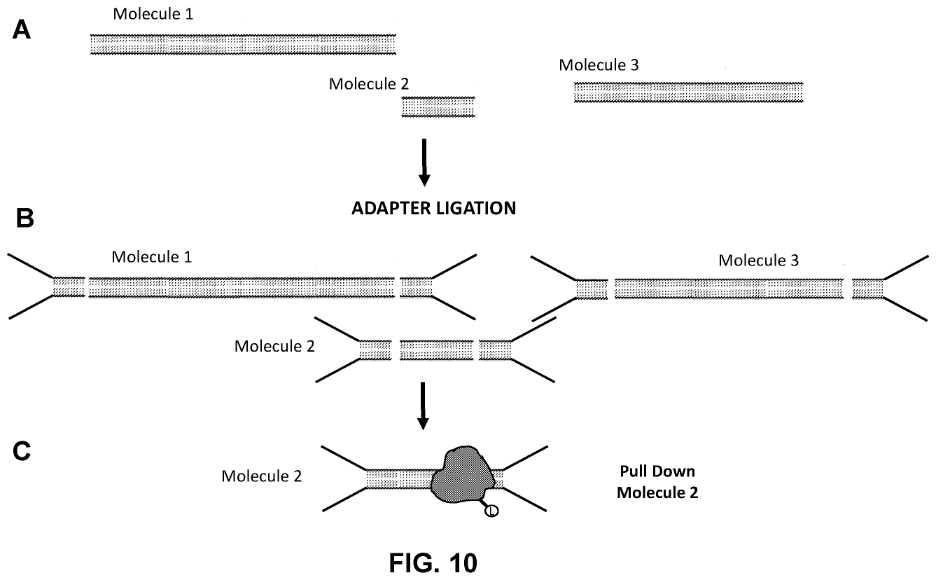

In some embodiments, it is desirable to enrich or isolate target nucleic acid material from a sample when the sample contains fragments of varying sizes, including fragment sizes that are small and might otherwise be lost during processing steps (e.g., DS process steps). FIG. 10 is a schematic illustrating methods steps for positive enrichment/selection of target nucleic acid fragments using a catalytically inactive variant of Cas 9 ribonucleoprotein complex bearing a capture label. Panel A illustrates a plurality of fragmented double-stranded DNA fragments of varying size in a sample, including Molecule 2 which is too small to reliably enrich via size selection or affinity-based methods. In this embodiment, adapters (e.g., sequencing adapters) can be ligated/attached to fragment ends using known sequencing library preparation steps. In this manner, certain small nucleic acid fragments are elongated by way of the flanking adapter molecules. Positive enrichment of the targeted fragments from solution can proceed as described above with respect to FIGS. 9A and 9B. For example, FIG. 10, Panel B illustrates ligating adapters to the 5′ and 3′ ends of the molecules in the sample, thereby making such DNA fragments longer in length. Panel C illustrates a positive enrichment/selection step of molecule 2 via target directed binding by a catalytically inactive Cas9 ribonucleoprotein complex bearing a capture label in solution followed by affinity purification.

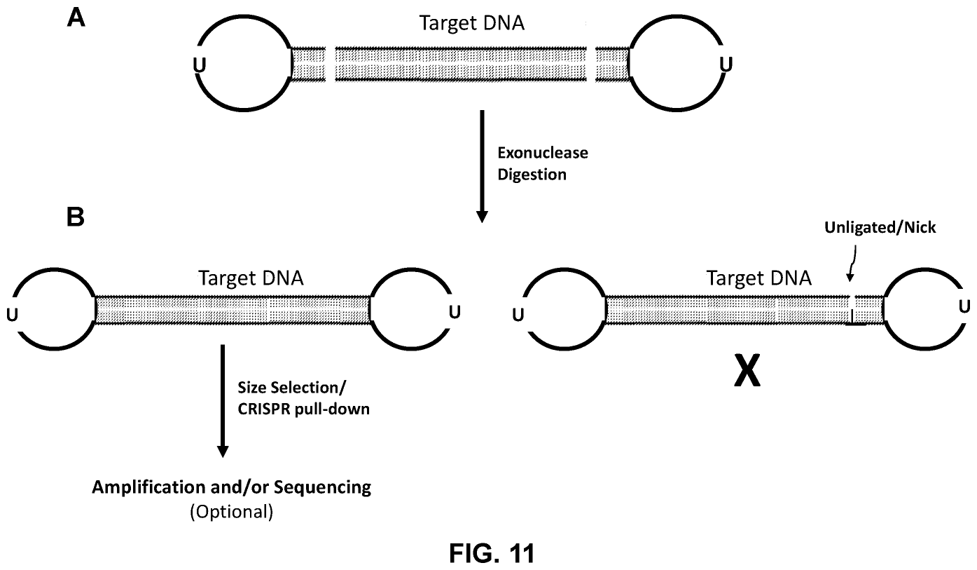

FIG. 11 is a schematic illustrating steps of a method for enriching targeted nucleic acid material using a negative enrichment scheme (Panel A) and a positive enrichment scheme (Panel B) in accordance with an embodiment of the present technology. Panel A shows ligation of hairpin adapters to the 5′ and 3′ ends of a double-stranded target DNA molecule to generate adapter-nucleic acid complexes with no exposed ends. The adapter-nucleic acid complexes are treated with exonuclease in a negative enrichment/selection scheme to eliminate nucleic acid material fragments and adapters with unprotected 5′ and 3′ ends (e.g., adapter-nucleic acid complexes without 4 ligated phosphodiester bonds, unligated DNA, single stranded nucleic acid material, free adapters, etc.) as illustrated on the right side of Panel B.

As shown in FIG. 11, the hairpin adapters can comprise a cleavable moiety, such as a uracil group, or any other enzymatically, chemically or photo-electrically cleavable group, in a linker portion. When treated with a combination of uracil DNA glycosylase (UDG) and an enzyme with abasic site DNA lyase activity such as endonuclease VIII or formamidopyrimidine [fapy]-DNA glycosylase (FPG) or commercial premixed combinations (for example USER™ enzyme), the cleavage at the uracil can transition the hairpin adapters to adapters comprising a Y-shape suitable for polony formation (bridge amplification) and certain sequencing modalities.

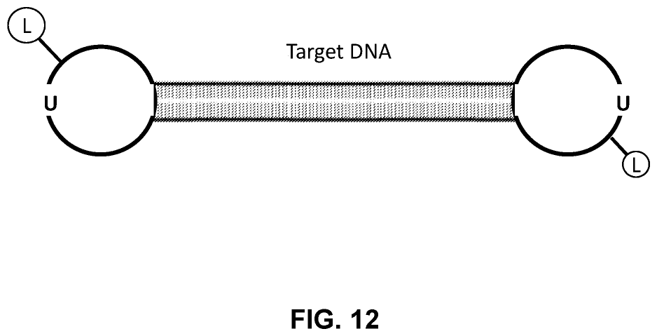

Exonuclease resistant adapter-nucleic acid complexes can be further enriched via size selection or via target sequence (e.g., CRISPR/Cas9 pull-down) (FIG. 11, Panel B, left side). In another embodiment, the hairpin adapters bearing a capture label can used (as shown in FIG. 12), which are directly suitable for affinity-based enrichment using functionalized surfaces with exposed extraction moieties.

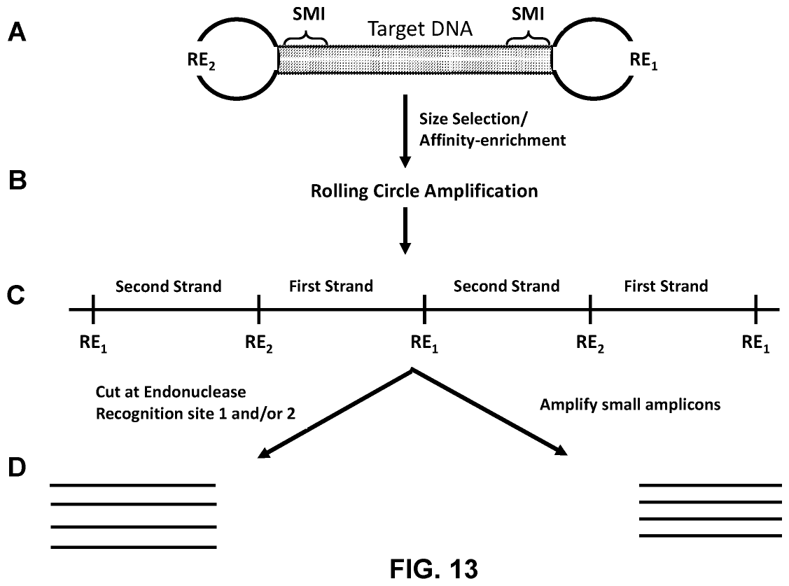

In embodiments following negative enrichment of target nucleic acid fragments ligated to hairpin adapters described in FIG. 11, additional positive enrichment steps can be performed. For example, FIG. 13 is a schematic illustrating method steps for positive enrichment of an adapter-target nucleic acid complex using hairpin adapters (Panel A) followed by rolling circle amplification (Panels B and C). Rolling circle amplification steps can be used to (1) provide substantially a 1:1 ration of first strand amplicons to second strand amplicons, and (2) prevent strand dissociation before tagging and/or during library clean up steps. Long molecule sequencing platforms can be suitable for directly sequencing the rolling circle amplicon (Panel C); however, for short read sequencing platforms, one can either (1) enzymatically cleave hairpin linker segments comprising a cleavage site (e.g., restriction endonuclease recognition site) to generate approximately even proportions of first strand and second strand amplicons (Panel D, left side), or (2) use PCR amplification to generate a plurality of short amplicons comprising first and second sequences (Panel D, right side) in substantially the same ratio.

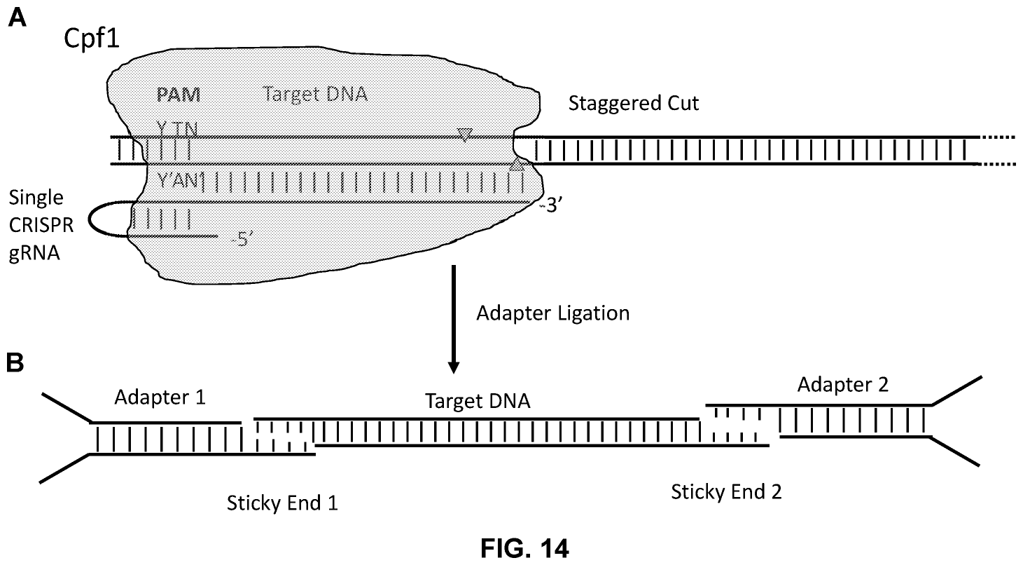

FIG. 14 is a schematic illustrating steps of a method for generating targeted nucleic acid fragments with known/selected length with different 5′ and 3′ ligatable ends using site-directed binding and cleavage of CRISPR/Cpf1. In various embodiments, the 5′ and 3′ ligatable ends comprise single-stranded overhang regions with known nucleotide length and sequence. Cpf1 in a targeted endonuclease that recognizes a T-rich PAM on the 5′ side of the guide and makes a staggered cut in the double-stranded DNA target sequence. For example, variants of Cpf1 cut 19 bp after the PAM on the sense strand and 23 bp on the antisense strand as shown in FIG. 14. Panel A illustrates gRNA-facilitated binding of Cpf1 at the targeted DNA site. Cpf1 directed cleavage generates the staggered cut providing a 4 (depicted) or 5 nucleotide overhang (e.g., “sticky end”). Site directed Cpf1 cleavage flanking a target DNA sequence, generates a double-stranded target DNA fragment of known length (e.g., which can be further and optionally enriched via size selection) with sticky end 1 at the 5′ end and sticky end 2 at the 3′ end of the fragment (Panel B). Panel B further illustrates attaching adapter 1 at the 5′ end and adapter 2 at the 3′ end of the fragment, wherein adapters 1 and 2 comprise at least partially complementary overhang sequences to sticky ends 1 and 2 on the fragment, respectively.

By design the sequence of sticky end 1 (overhang at the 5′ end of the targeted fragment) is known. Likewise, the sequence of sticky end 2 (overhang at the 3′ end of the targeted fragment) is known. Specific adapters comprising substantially complementary sequences can be synthesized such that fragments can be attached to adapter at both ends. In one embodiment, the adapters can be the same type of adapters (e.g., adapters comprising a Y-shape, U-shape, barcoded adapters, etc.). In another embodiment the adapters can be different (e.g., adapter 1 can comprise a Y-shape and adapter 2 can comprise a U-shape). Other unique features may include different primer sites for amplification, different types or locations of barcodes or other unique molecular identifiers, adapters comprising capture labels and ones without capture labels, certain adapters can comprise fluorescent tags and the like. There are identified advantages in some applications to designing specific adapters to be positioned in either the 5′ or 3′ ends of fragments. The specificity of substantially unique sticky ends on the targeted fragments facilitates these types of applications. Moreover, positive selection of successfully cleaved and adapter ligated target fragments can ensure only amplification and sequencing of the target enriched nucleic acid regions.

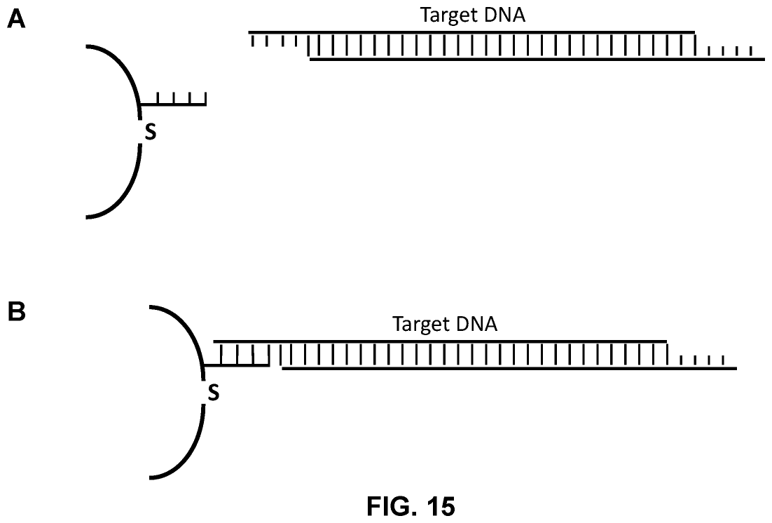

In some embodiments, the substantially unique sticky ends generated by Cpf1 cleavage can be used in additional positive enrichment schemes. For example, FIG. 15 is a schematic illustrating steps of a method for affinity-based enrichment of a target DNA fragment comprising sticky end(s) (e.g., such as target DNA fragments generated in the method of FIG. 14) in accordance with an embodiment of the present technology. Panel A illustrates step-wise addition of a functionalized surface that is capable of binding a sticky end associated with the cut target DNA fragment in solution. For example, the functionalized surface can have one or more extraction moieties bound thereto suitable as a binding pair to one or more targeted DNA overhang sequences. The provided extraction moieties can be, for example, synthesized oligonucleotides with pre-defined or known oligonucleotide sequence at least partially complementary to the generated sticky end(s) of the Cpf1 cleaved target sequences. The oligonucleotides can comprise DNA, RNA or LNA sequences capable of binding to the capture label (e.g. the sticky end) for immobilization and separation of the target comprising the sticky end(s). Once bound to the functionalized surface, the affinity interaction facilitates pull-down (e.g., affinity purification) of the desired double-stranded DNA fragment while discarding non targeted fragments as shown in Panel B.

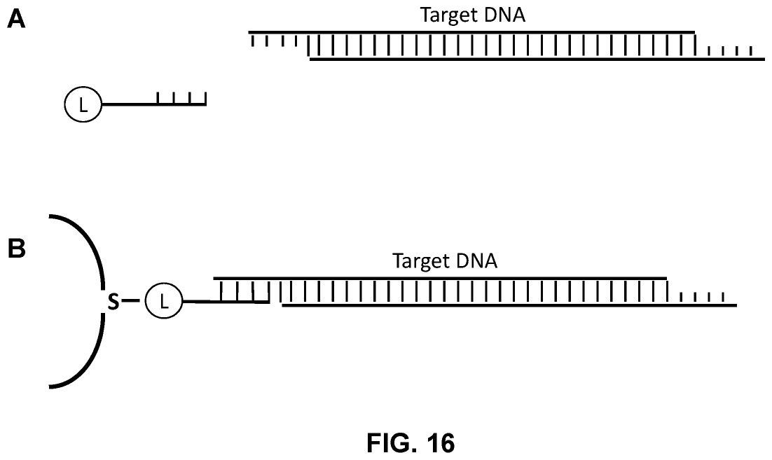

FIG. 16 is a schematic illustrating steps of a method for affinity-based enrichment of a target DNA fragment comprising sticky end(s) (e.g., such as target DNA fragments generated in the method of FIG. 14) in accordance with another embodiment of the present technology. Panel A illustrates step-wise addition of a capture label-bearing oligonucleotide having a pre-defined or known oligonucleotide sequence at least partially complementary to at a portion of a sticky end associated with the cut target DNA fragment in solution. In a particular example, oligonucleotide strands can be synthesized (e.g., on controlled pore glass (CPG) fragments or the like) in a 3′ to 5′ direction such as via the phosphoramidite method, and a chemical moiety can be linked (e.g., covalently linked, non-covalently linked, ionically linked or other linking chemistry) to the 5′ terminus following synthesis of the oligonucleotide, or as part of the synthesis of the oligonucleotide, such as via incorporation of a non-canonical phosphoramidite molecule at the 5′ terminus, near the 5′ terminus or at an internal position in the oligonucleotide.

As shown in Panel B, further addition of a functionalized surface that is capable of binding the capture label facilitates pull-down (e.g., affinity purification) of the desired double-stranded DNA fragment while discarding non targeted fragments.

Referring to FIGS. 15 and 16 together, and in next steps (not shown) elution of the targeted fragments can occur via release from the extraction moieties. In some non-limiting examples, a cleavable moiety can be incorporated proximate the bound end of the oligonucleotide extraction moiety. In another embodiment, temperature or other conditions can be changed to cause denaturing of the short capture label/extraction binding while maintaining the double-stranded nature of the target nucleic acid fragment. In still another embodiment, hairpin adapters can be used at a second sticky end of the target fragments to tether the duplex strands together during elution and further processing. In various embodiments, after enrichment steps, the sticky ends can be polished, trimmed or biocomputationally filtered as described herein for avoiding pseudoplex errors.

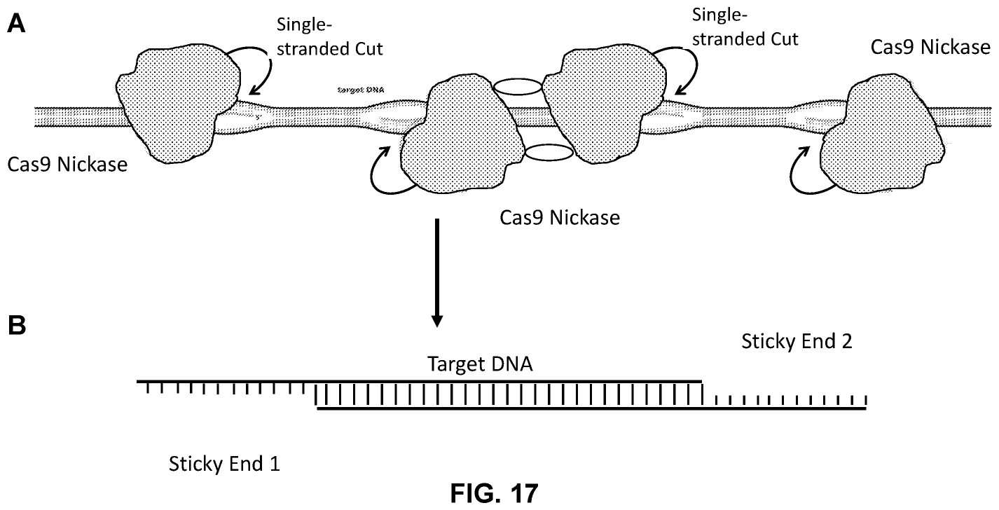

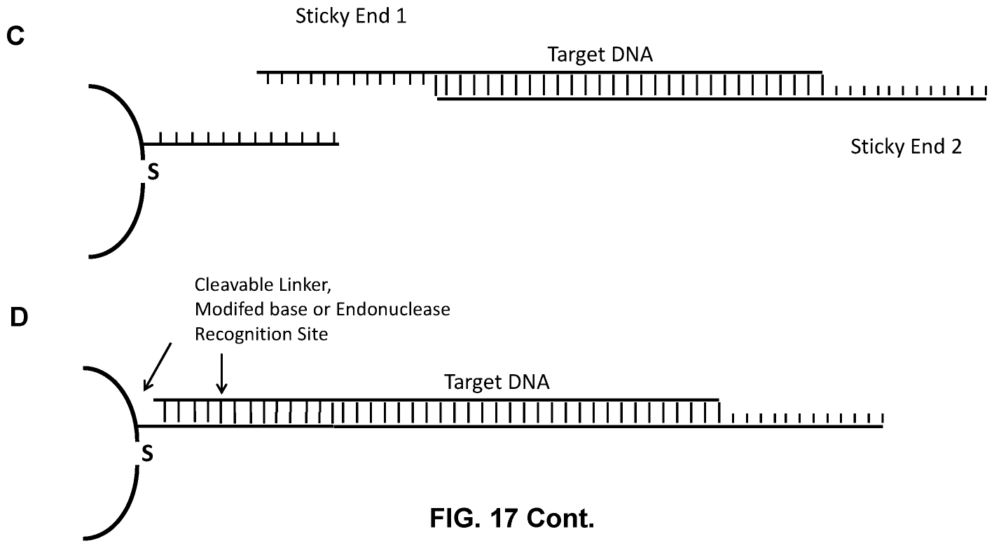

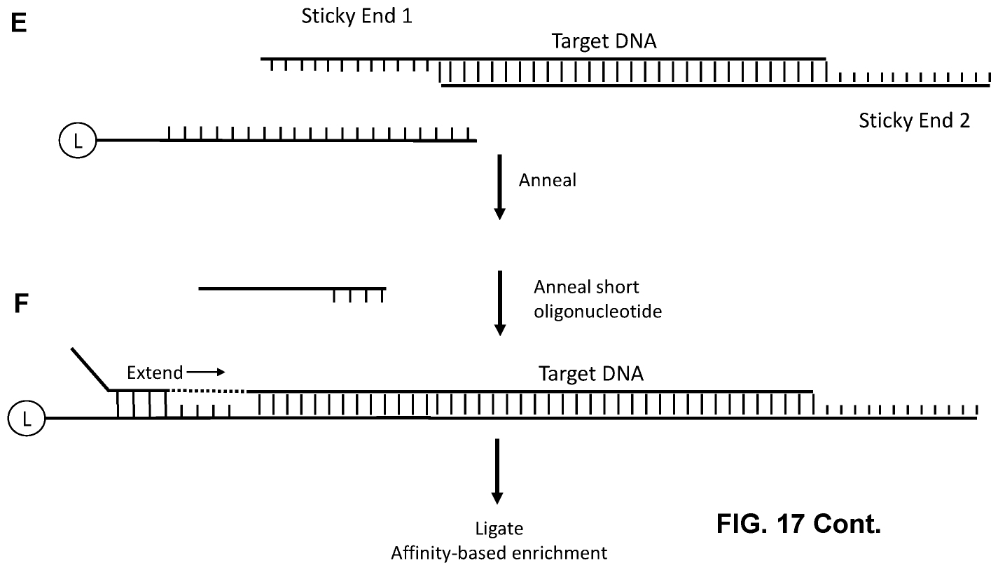

FIG. 17 is a schematic illustrating steps of a method for targeted fragment enrichment of nucleic acid material having a known length and having different 5′ and 3′ ligatable ends comprising long single-stranded overhang regions with known nucleotide length and sequence using Cas9 Nickase and in accordance with an embodiment of the present technology. Panel A illustrates gRNA targeted binding of paired Cas9 nickases in a targeted DNA region. Double-strand breaks can be introduced through the use of paired nickases to excise the target DNA region and, when paired Cas9 nickases are used, long overhangs (sticky ends 1 and 2) are produced on each of the cleaved ends as illustrated in Panel B. Accordingly, in contrast to cleavage with catalytically active Cas9, which produces blunt ends, strategic pairing of Cas9 nickases can provide staggered single strand cuts on opposing DNA strands to produce long overhangs as depicted in Panel B. As described above with respect to FIG. 15, step-wise addition of a functionalized surface that is capable of binding a long sticky end (e.g., sticky end 1) associated with the cut target DNA fragment in solution provides a positive enrichment step for the targeted DNA fragments in solution. For example, the extraction moiety can be an oligonucleotide having a pre-defined or known oligonucleotide sequence substantially complementary to the pre-defined or known sequence of the long sticky end of the fragment. Once bound to the functionalized surface, the affinity interaction facilitates pull-down (e.g., affinity purification) of the desired double-stranded DNA fragment while discarding non targeted fragments as shown in Panel D.

FIG. 17, Panel E illustrates a variation of a positive enrichment step comprising addition and annealing of a capture label-bearing oligonucleotide having a pre-defined or known oligonucleotide sequence at least partially complementary to at a portion of a long sticky end (e.g., sticky end 1) associated with the cut target DNA fragment in solution. Panel F illustrates annealing of a second oligo strand at least partially complementary to a portion of the capture label-bearing oligonucleotide. Enzymatic extension of the second oligo strand and ligation to the template DNA fragment generates an adapter-target DNA complex. As illustrated, the first and second oligonucleotide strands comprise single-stranded portions such that the resultant adapter complex comprises asymmetry for DS processing. Further the first oligonucleotide strand can comprise a degenerate or semi-degenerate SMI sequence such that when the second oligonucleotide strand elongates, the first oligonucleotide strand functions a template strand and the SMI sequence is made double-stranded. Further steps can include introduction of a functionalized surface (not shown) that is capable of binding the capture label to facilitate pull-down (e.g., affinity purification) of the desired adapter-double-stranded DNA complex while discarding non targeted fragments.

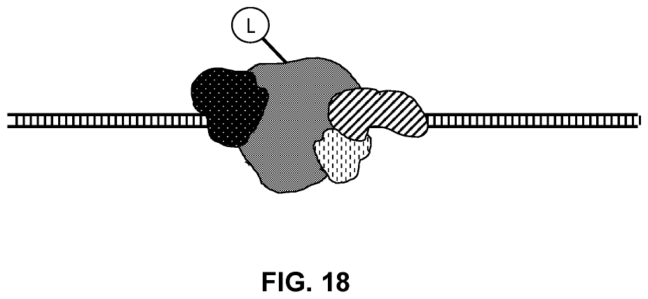

Various aspects of the present technology include methods for negatively enriching nucleic acid regions by providing exo- and endo-nuclease resistance by way of protein binding. In one embodiment, illustrated in FIG. 18, site selected protein binding to target DNA can be used to provide exo- and endo-nuclease resistance. As illustrated, a target nucleic acid enrichment scheme uses catalytically inactive Cas9 ribonucleoprotein complexes to protect targeted genomic regions. Cas9, by way of gRNA, can be targeted to desired sequences in a sample. One or more catalytically inactive ribonucleoprotein complexes bearing one or more capture labels can be positioned in close proximity and/or adjacently to protect regions of genomic DNA from enzymatic digestion. In some embodiments, as shown, the ribonuclease complex can be engineered to direct other protein complex structures to the target DNA region. Where the protein complex structure covers the target DNA region, exonuclease resistance is provided. Following treatment with an exonuclease or a combination of endonucleases and exonucleases, affinity purification of the protein complex (e.g., via a capture label binding to a functionalized surface, antibody pull-down, etc.) separates the target DNA fragments from other undesired nucleic acid material or unbound proteins in solution. The target nucleic acid fragment can then be released from ribonucleotide complex binding

Nucleic Acid Libraries and Methods for Making and Using Nucleic Acid Libraries

In some embodiments, a provided method may include the steps of providing a nucleic acid material, directing a plurality of targeted catalytically inactive endonucleases (e.g., a ribonucleoprotein complexes) to a plurality of regions disbursed along the nucleic acid material to create a nucleic acid library that can be interrogated via selective probes at any time

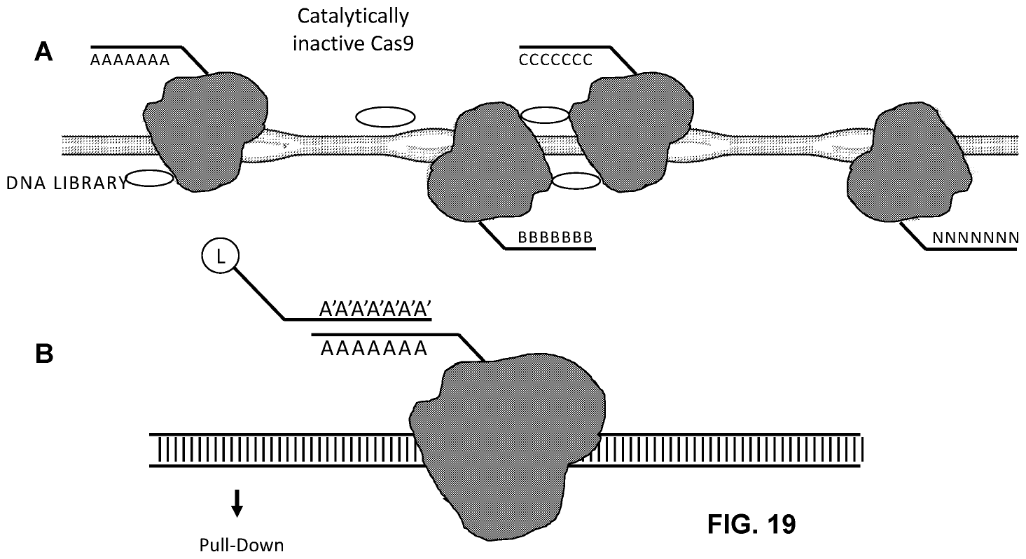

FIGS. 19A and 19B are conceptual illustrations of a prepared DNA library and reagents that can be used as a tool to selectively interrogate DNA regions of interest in accordance with an embodiment of the present technology. Uniquely tagged catalytically inactive Cas9 is target directed to multiple (e.g., interspaced) regions of isolated/unfragmented genomic DNA (or other large fragments of DNA) (FIG. 19A). Each catalytically inactive Cas9 ribonucleoprotein comprises a known oligonucleotide tag with known sequence (e.g., a code sequence) and is bound to a pre-designed region of a genome. As schematically illustrated in FIG. 19A, a plurality of inactive Cas9 ribonucleoprotein complexes (e.g., iCas9^{A}, iCas9^{B}, iCas9^{C}, iCas9^{N}) are gRNA-directed to bind genomic sites (Site^{A}, Site^{B}, Site^{C}, Site^{N}) disbursed throughout a genomic region (e.g., a large selected region, an entire genome, etc.). Each iCas9 complex comprises an oligonucleotide tag comprising an oligonucleotide code sequence (AAAAAAA), where “A” is any nucleotide (unmodified or modified) the sting of nucleotides comprises a substantially unique code that can be recorded and later looked up in a look-up table.

When desirable to interrogate (e.g., sequence) a particular target sequence or smaller region, the library can be probed with specifically designed capture probes engineered to pulldown the desired region. A method of fragmentation can be used to fragment the genomic DNA in various sizes (e.g., restriction enzymatic digestion, mechanical shearing, etc.). As each of the iCas9 complexes comprise a substantially unique oligonucleotide tag that is computationally associated with the DNA site, a user can step-wise add one or more probes comprising the compliment of the code sequence corresponding to the region of the genome of interest (e.g., an anticode sequence). For example, and as shown in FIG. 19B, an anticode sequence is a nucleotide sequence substantially complementary to the codes sequence of interest. For example, to extract a region comprising site^{A}, a user looks up the code sequence associated with the iCas9A complex bound to site^{A} (AAAAAAA). Then, using an oligonucleotide probe comprising a capture label affixed or incorporated thereto and comprising an anticode sequence (A′A′A′A′A′A′A′), the regions of interest can be functionally selected and enriched via introduction of a functionalized surface bearing an appropriate extraction moiety (e.g., streptavidin where biotin is the capture label).

In various embodiments, the nucleic acid library can be used as a resource for several probed interrogations. Additionally, several libraries can be prepared having multiple CRISPR/Cas site-directed complexes pre-bound thereto. Further, some libraries can be pre-fragmented or cut using either mechanical shearing, endonuclease cutting (using one or more restriction endonucleases). When the desired target region is excised (e.g., via targeted endonuclease digestion (e.g., CRISPR/Cas, restriction enzyme, etc.), the length of the target fragment will be known and following pull-down using the probes, the target fragments can be further enriched via size selection.

Additional Methods

Some aspects of the present technology are suitable for use with long sequence sequencing technologies, such as direct digital sequencing (DDS) platforms. In some embodiments, it is desirable to enrich for target sequences of interest for use with DDS. In such embodiments, it is desirable to do amplification-free enrichment for target sequences. Additionally, it is further desirable to generate duplex sequencing data on such platforms.

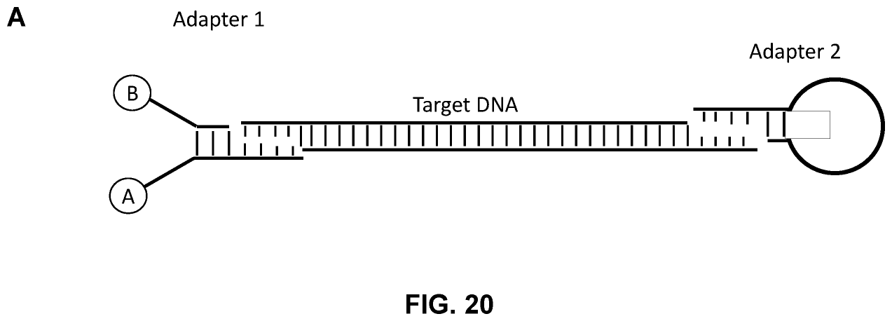

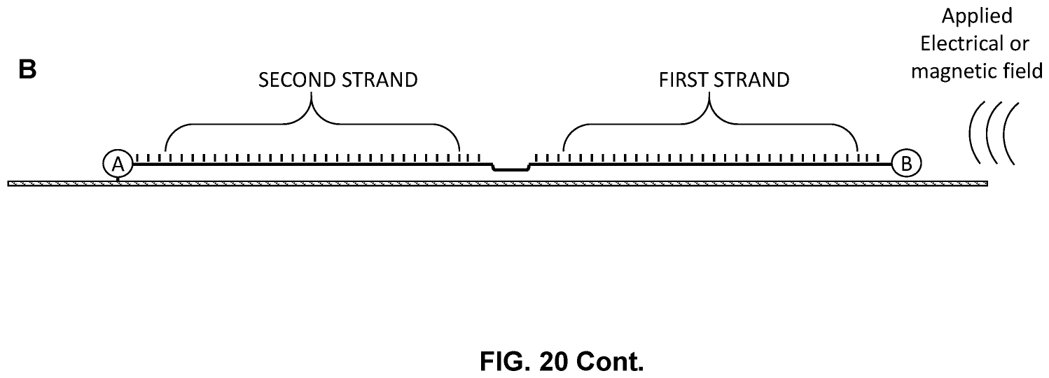

FIG. 20 illustrates a step of a method for affinity-based enrichment and sequencing of a target DNA fragment for use with a direct digital sequencing method in accordance with an embodiment of the present technology. Panel A shows selected adapter attachment to a target DNA fragment comprising sticky end(s) (e.g., such as target DNA fragments generated in the method of FIG. 14 or FIG. 17). Panel A further illustrates attaching adapter 1 at the 5′ end and adapter 2 at the 3′ end of the fragment, wherein adapters 1 and 2 comprise at least partially complementary overhang sequences to sticky ends 1 and 2 on the fragment, respectively. Adapter 1 has a Y-shape and comprises 5′ and 3′ single-stranded arms bearing different labels (A and B) comprising different properties. Adapter 2 is a hairpin-shaped adapter.

Panel B illustrates a step in a direct digital sequencing method where label A is configured to be bound to a functional surface. Label B provides a physical property (e.g., electric charge, magnetic property, etc.) such that application of an electrical or magnetic field causes denaturation of the first and second strands of the double-stranded adapter-DNA complex followed by electro-stretching of the DNA fragment. The first and second strands remain tethered by the hairpin adapter such that sequence information from the enriched/targeted strand provides duplex sequence information for error-correction and other nucleic acid interrogation (e.g., assessment of DNA damage, etc.). For example, a sequence generated from the first strand can be compared to a sequence compared to the second strand for error-correction, or in another example, to determine sites and characteristics of DNA damage In some embodiments, the targeted genomic region that is enriched can have lengths from between about 1 and 1,000,000 bases. For example, in some embodiments, and when denatured and sequenced, a length of an enriched nucleic acid fragment may be at least 1; 2; 3; 4; 5; 6; 7; 8; 9; 10; 15; 20; 25; 30; 35; 40; 50; 60; 70; 80; 90; 100; 120; 150; 200; 300; 400; 500; 600; 700; 800; 900; 1000; 1200; 1500; 2000; 3000; 4000; 5000; 6000; 7000; 8000; 9000; 10,000; 15,000; 20,000; 30,000; 40,000; or 50,000 bases in length. In some embodiments, a length of the fragment may be at most 60,000; 70,000; 80,000; 90,000; 100,000; 120,000; 150,000; 200,000; 300,000; 400,000; 500,000; 600,000; 700,000; 800,000; 900,000; or 1,000,000 bases.

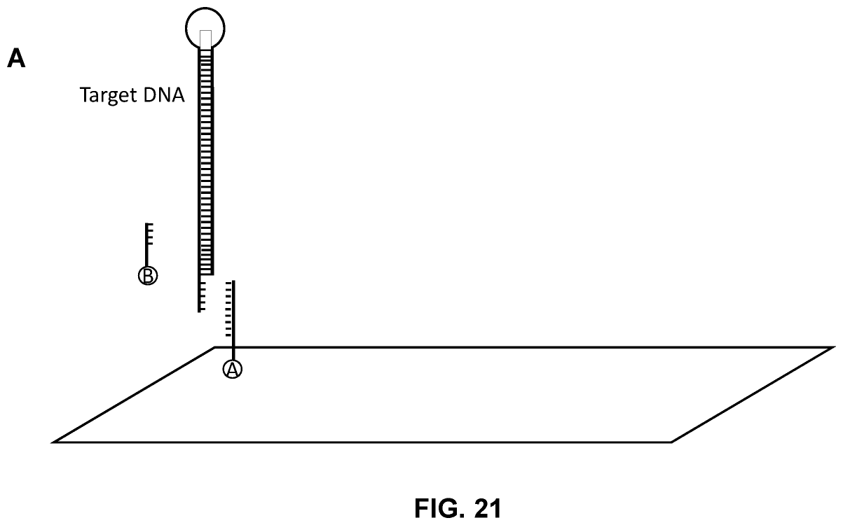

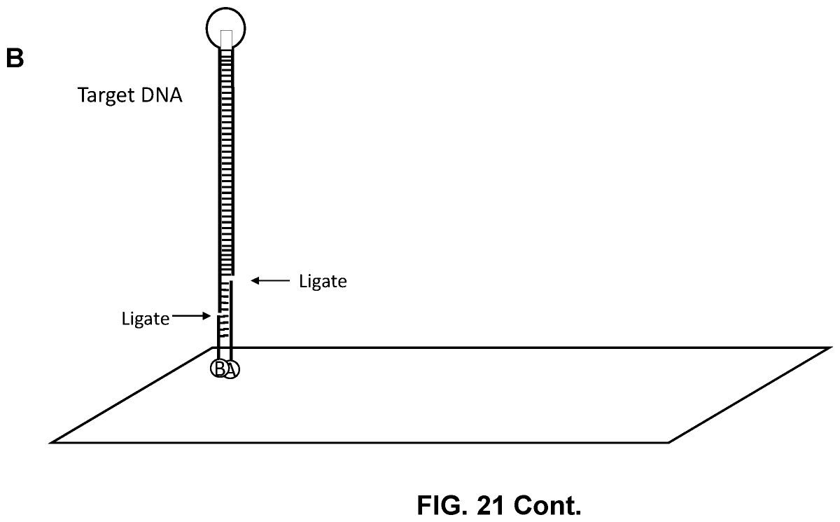

FIG. 21 illustrates a step of a method for affinity-based enrichment for sequencing of a target DNA fragment using a DDS method in accordance with another embodiment of the present technology. Panel A shows affinity-based enrichment of a target DNA fragment comprising sticky end(s) (e.g., such as target DNA fragments generated in the method of FIG. 14 or FIG. 17). As illustrated, a hairpin adapter has been attached to a 3′ end of the double-stranded DNA fragment in a sequence-dependent manner. The target DNA molecule(s) can be flowed over a functionalized surface capable of binding a sticky end associated with the cut target DNA fragment (e.g., having bound oligonucleotides). Additionally, a second oligonucleotide strand comprising label B and at least partially complementary to a portion of the bound oligonucleotide is added into solution. Annealing and ligation of the adapter/DNA fragment components provides an adapter-target double-stranded DNA complex bound to a surface suitable for direct digital sequencing (Panel B). Application of an electrical or magnetic field and electro-stretching of the adapter-DNA complex for sequencing steps can occur as described, for example, in FIG. 20.

Reagents and Methods

Adapter Types

While the majority of examples in the present disclosure depict Y shaped or loop adapters, any known adapter structure may be used in accordance with various embodiments, such as those described in WO2017/100441, which is incorporated herein by reference in its entirety. For example, various adapter shapes comprising bubbles (e.g., internal regions of non-complementarity) are further contemplated.

Separation

As is described herein, various methods include at least one separation step. It is specifically contemplated that any of a variety of separation steps may be included in various embodiments. For example, in some embodiments, separation may be or comprise physical separation, size separation, magnetic separation, solubility separation, charge separation, hydrophobicity separation, polarity separation, electrophoretic mobility separation, density separation, chemical elution separation, SBIR bead separation etc. For example, a physical group can have a magnetic property, a charge property, or an insolubility property. In embodiments, when the physical group has a magnetic property and a magnetic field is applied, the associated adapter nucleic acid sequences including the physical group is separated from the adapter nucleic acid sequences not including the physical group. In another embodiment, when the physical group has a charge property and an electric field is applied, the associated adapter nucleic acid sequences including the physical group is separated from the adapter nucleic acid sequence not including the physical group. In embodiments, when the physical group has an insolubility property and the adapter nucleic acid sequences are contained in a solution for which the physical group is insoluble, the adapter nucleic acid sequences comprising the physical group is precipitated away from the adapter nucleic acid sequence not including the physical group which remains in solution.

Any of a variety of physical separation methods may be included in various embodiments. By way of specific example, a non-limiting set of methods includes: size selective filtration, density centrifugation, HPLC separation, gel filtration separation, FPLC separation, density gradient centrifugation and gel chromatography, among others.

Any of a variety of magnetic separation methods may be included in various embodiments. Typically, magnetic separation methods will encompass the inclusion or addition of one or more physical groups having a magnetic property such that, when a magnetic field is applied, molecules including such physical group(s) are separated from those that do not. By way of specific example, physical groups that include exhibit a magnetic property include, but are not limited to ferromagnetic materials such as iron, nickel, cobalt, dysprosium, gadolinium and alloys thereof. Commonly used paramagnetic beads for chemical and biochemical separation embed such materials within a surface that reduces chemical interaction of the materials with the chemicals being manipulated, such as polystyrene, which can be functionalized for the affinity properties discussed above.

Capture Labels

As is described herein, in some embodiments, a capture label may be present in any of a variety of configurations on proteins, along oligonucleotide probes, adapters, ribonucleotide sequences, ribonucleoprotein complexes, etc. In some embodiments, a capture label can be incorporated or affixed to an oligonucleotide strand in a region 5′ of the sequence. In some embodiments, a capture label may be present somewhere in the middle of an oligonucleotide strand (i.e., not on the 5′ or 3′ end of the oligonucleotide). In embodiments including two or more capture labels, each capture label may be present at a different location along the oligonucleotides.

In some embodiments, a capture label is selected from a group of biotin, biotin deoxythymidine dT, biotin NHS, biotin TEG, Biotin-6-Aminoaliyl-2′-deoxyuridine-S′-Triphosphate, Biotin-16-Aminoallyl-2-deoxycytidine-5′-Triphosphate, Biotin16-Aminoallylcytidine-5′-Triphosphate, N4-Biotin-OBEA-2′-deoxycytidine-5′-Triphosphate, Biotin-16-Aminoallyluridine-5′-Triphosphate, Biotin-16-7-Deaza-7-Aminoallyl-2′-deoxyguanosine-5′-Triphosphate, 5′-Biotin-G-Monophosphate, 5′-Biotin-A-Monophosphate, 5′-Biotin-dG-Monophosphate, 5′-Biotin-dA-Monophosphate, desthiobiotin NHS, Desthiobiotin-6-Aminoallyl-2′-deoxycytidine-5′-Triphosphate, digoxigenin NHS, DNP TEG, thiols, Colicin E2, Im2, glutathione, glutathione-s-transferase (GST), nickel, polyhistidine, FLAG-tag, myc-tag, among others. In some embodiments, capture labels include, without limitation, biotin, avidin, streptavidin, a hapten recognized by an antibody, a particular nucleic acid sequence and/or magnetically attractable particle. In some embodiments, one or more chemical modifications of nucleic acid molecules (e.g., Acridite™-modified among many other modifications, some of which are described elsewhere in the application) can serve as a capture label.

Extraction Moieties

Extraction moieties can be a physical binding partner or pair to targeted capture label and refers to an isolatable moiety or any type of molecule that allows affinity separation of nucleic acids bearing the capture label or bound by a capture label bearing molecule (e.g., oligonucleotide, protein, ribonucleoprotein complex, etc.) from nucleic acids lacking the capture label. Extraction moieties can be directly linked or indirectly linked (e.g., via nucleic acid, via antibody, via aptamer, etc.) to a substrate, such as a solid surface. In some embodiments, the extraction moiety is selected from a group comprising a small molecule, a nucleic acid, a peptide, an antibody or any uniquely bindable moiety. The extraction moiety can be linked or linkable to a solid phase or other surface for forming a functionalized surface. In some embodiments, the extraction moiety is a sequence of nucleotides linked to a surface (e.g., a solid surface, bead, magnetic particle, etc.). In some embodiments, wherein the capture label is biotin, the extraction moiety is selected from a group of avidin or streptavidin. It will be appreciated by one of skill in the art, any of a variety of affinity binding pairs may be used in accordance with various embodiments.

In certain embodiments, extraction moieties can be physical or chemical properties that interact with the targeted capture label. For example, an extraction moiety can be a magnetic field, a charge field or a liquid solution in which a targeted capture label is insoluble. Such physical or chemical properties can be applied and adapter nucleic acids bearing the capture label can be immobilized within/against a vessel (surface) or column. Depending on the desired positive enrichment/selection or negative enrichment/selection outcome, the immobilized molecules can be retained (positive enrichment) or the non-immobilized molecules can be retained (negative enrichment) for further purification/processing or use.

Solid Surfaces

When the affinity partner/extraction moiety is attached to a solid surface or substrate and bound to the capture label, the adapter nucleic acid sequences including the capture label is capable of being separated from the adapter nucleic acid sequence not including the affinity label. A solid surface or substrate may be a bead, isolatable particle, magnetic particle or another fixed structure.

As is described herein and will be appreciated by one of skill in the art, any of a variety of functionalized surfaces may be used in accordance with various embodiments. For example, in some embodiments, a functionalized surface may be or comprise a bead (e.g., a controlled pore glass bead, a macroporous polystyrene bead, etc.). However, it will be understood to one of skill in the art that many other chemical moiety/surface pairs could be similarly used to achieve the same purpose. It will be understood that the specific functionalized surfaces described here are meant only as examples, and that any other appropriate fixed structure or substrate capable of being associated with (e.g., linked to, bound to, etc.) one or more extraction moieties may be used.

Cutting of Nucleic Acids

Various aspects of the present technology, including the enrichment of nucleic acid material using adapters, oligonucleotides and capture labels that may incorporate enzymatic cleavage, enzymatic cleavage of a single strand, enzymatic cleavage of double strands, incorporation of a modified nucleic acid followed by enzymatic treatment that leads to cleavage or one or both strands, incorporation of a photocleavable linker, incorporation of a uracil, incorporation of a ribose base, incorporation of an 8-oxo-guanine adduct, use of a restriction endonuclease, use of site-directed cutting enzymes, and the like. In other embodiments, endonucleases, such as a ribonucleoprotein endonuclease (e.g., a Cas-enzyme, such as Cas9 or CPF1), or other programmable endonuclease (e.g., a homing endonuclease, a zinc-fingered nuclease, a TALEN, a meganuclease (e.g., megaTAL nuclease), an argonaute nuclease, etc.), and any combination thereof can be used.

As is described herein, various embodiments include the use of one or more endonucleases which recognize unique nucleotide sequences or modifications or other entities that recognizes base or other backbone chemical modifications for cutting and/or cleaving a double stranded nucleic acid (e.g., DNA or RNA) at a specific location in one or more strands. Examples include Uracil (recognized and can be cleaved with a combination of Uracil DNA glycosylase and an abasic site lyase such as Endonuclease VIII or FPG, and ribose nucleotides, which can be recognized and cleaved by RNAseH2 when these are paired with DNA base. The nucleic acid may be DNA, RNA, or a combination thereof, and optionally, including a peptide-nucleic acid (PNA) or a locked nucleic acid (LNA) or other modified nucleic acid. In some embodiments, cutting may be performed via use of one or more restriction endonucleases. In some embodiments, cleaving may be performed using a cleavable linker, for example, uracil desthiobotin-TEG, ribose cleavage, or other methods. In some embodiments the cleavable linker may be a photocleavable linker or a chemical cleavable linker not requiring of enzymes, or partially.

It will be appreciated by one of ordinary skill in the art that a variety of restriction endonucleases (i.e., restriction enzymes) that cleaves DNA at or near recognition sites (e.g., EcoRI, BamHI, XbaI, HindIII, AluI, AvaII, BsaJI, BstNI, DsaV, Fnu4HI, HaeIII, MaeIII, N1aIV, NSiI, MspJI, FspEI, NaeI, Bsu36I, NotI, HinFI, Sau3AI, PvuII, SmaI, HgaI, AluI, EcoRV, etc.) may be in accordance with various embodiments of the present technology. Listings of several restriction endonucleases are available both in printed and computer readable forms, and are provided by many commercial suppliers (e.g., New England Biolabs, Ipswich, Mass.). A non-limiting list of restriction endonucleases and associated recognition sites may be found at: www. .neb.com/tools-and-resources/selection-charts/alphabetized-list-of-recognition-specificities.

In some embodiments, modified or non-nucleotides can provide a cleavable moiety. For example, uracil bases (can be cleaved with combination of UGD and endonuclease VIII or FPG as one example), abasic sites (can be cleaved by Endonuclease VIII as one example), 8-oxo-guanine (can be cleaved by FPG or OGG1 as examples) and ribose nucleotides (can be cleaved by RNAseH2 in when paired with DNA in one example).

Ligateable Ends

In some embodiments, adapter products are generated with a ligateable 3′ end suitable for ligation to target double-stranded nucleic acid sequences (e.g., for sequencing library preparation). Ligation domains present in each of the double-stranded adapter products may be capable of being ligated to one corresponding strand of a double-stranded target nucleic acid sequence. In some embodiments, one of the ligation domains includes a T-overhang, an A-overhang, a CG-overhang, a multiple nucleotide overhang, a blunt end, or another ligateable nucleic acid sequence. In some embodiments, a double-stranded 3′ ligation domain comprises a blunt end. In certain embodiments, at least one of the ligation domain sequences includes a modified or non-standard nucleic acid. In some embodiments, a modified nucleotide may be an abasic site, a uracil, tetrahydrofuran, 8-oxo-7,8-dihydro-2′-deoxyadenosine (8-oxo-A), 8-oxo-7,8-dihydro-2′-deoxyguanosine (8-oxo-G), deoxyinosine, 5′-nitroindole, 5-Hydroxymethyl-2′-deoxycytidine, iso-cytosine, 5′-methyl-isocytosine, or iso-guanosine. In some embodiments, at least one strand of the ligation domain includes a dephosphorylated base. In some embodiments, at least one of the ligation domains includes a dehydroxylated base. In some embodiments, at least one strand of the ligation domain has been chemically modified so as to render it unligateable (e.g., until a further action is performed to render the ligation domain ligateable). In some embodiments a 3′ overhang is obtained by use of a polymerase with terminal transferase activity. In one example Taq polymerase may add a single base pair overhang. In some embodiments this is an “A”.

Non-Standard Nucleotides

In some embodiments, provided template and/or elongation strands may include one or more non-standard/non-canonical nucleotides. In some embodiments, a non-standard nucleotide may be or comprise a uracil, a methylated nucleotide, an RNA nucleotide, a ribose nucleotide, an 8-oxo-guanine, a biotinylated nucleotide, a desthiobiotin nucleotide, a thiol modified nucleotide, an acrydite modified nucleotide an iso-dC, an iso dG, a 2′-O-methyl nucleotide, an inosine nucleotide Locked Nucleic Acid, a peptide nucleic acid, a 5 methyl dC, a 5-bromo deoxyuridine, a 2,6-Diaminopurine, 2-Aminopurine nucleotide, an abasic nucleotide, a 5-Nitroindole nucleotide, an adenylated nucleotide, an azide nucleotide, a digoxigenin nucleotide, an I-linker, a 5′ Hexynyl modified nucleotide, an 5-Octadiynyl dU, photocleavable spacer, a non-photocleavable spacer, a click chemistry compatible modified nucleotide, a fluorescent dye, biotin, furan, BrdU, Fluoro-dU, Ioto-dU, and any combination thereof.

Additional Aspects

In accordance with an aspect of the present disclosure some embodiments provide high quality sequencing information from very small amounts of nucleic acid material. In some embodiments, provided methods and compositions may be used with an amount of starting nucleic acid material of at most about: 1 picogram (pg); 10 pg; 100 pg; 1 nanogram (ng); 10 ng; 100 ng; 200 ng, 300 ng, 400 ng, 500 ng, 600 ng, 700 ng, 800 ng, 900 ng, or 1000 ng. In some embodiments, provided methods and compositions may be used with an input amount of nucleic acid material of at most 1 molecular copy or genome-equivalent, 10 molecular copies or the genome-equivalent thereof, 100 molecular copies or the genome-equivalent thereof, 1,000 molecular copies or the genome-equivalent thereof, 10,000 molecular copies or the genome-equivalent thereof, 100,000 molecular copies or the genome-equivalent thereof, or 1,000,000 molecular copies or the genome-equivalent thereof. For example, in some embodiments, at most 1,000 ng of nucleic acid material is initially provided for a particular sequencing process. For example, in some embodiments, at most 100 ng of nucleic acid material is initially provided for a particular sequencing process. For example, in some embodiments, at most 10 ng of nucleic acid material is initially provided for a particular sequencing process. For example, in some embodiments, at most 1 ng of nucleic acid material is initially provided for a particular sequencing process. For example, in some embodiments, at most 100 pg of nucleic acid material is initially provided for a particular sequencing process. For example, in some embodiments, at most 1 pg of nucleic acid material is initially provided for a particular sequencing process.

In accordance with other aspects of the present technology, some provided methods may be useful in sequencing any of a variety of suboptimal (e.g., damaged or degraded) samples of nucleic acid material. For example, in some embodiments at least some of the nucleic acid material is damaged In some embodiments, the damage is or comprises at least one of oxidation, alkylation, deamination, methylation, hydrolysis, nicking, intra-strand crosslinks, inter-strand cross links, blunt end strand breakage, staggered end double strand breakage, phosphorylation, dephosphorylation, sumoylation, glycosylation, single-stranded gaps, damage from heat, damage from desiccation, damage from UV exposure, damage from gamma radiation damage from X-radiation, damage from ionizing radiation, damage from non-ionizing radiation, damage from heavy particle radiation, damage from nuclear decay, damage from beta-radiation, damage from alpha radiation, damage from neutron radiation, damage from proton radiation, damage from cosmic radiation, damage from high pH, damage from low pH, damage from reactive oxidative species, damage from free radicals, damage from peroxide, damage from hypochlorite, damage from tissue fixation such formalin or formaldehyde, damage from reactive iron, damage from low ionic conditions, damage from high ionic conditions, damage from unbuffered conditions, damage from nucleases, damage from environmental exposure, damage from fire, damage from mechanical stress, damage from enzymatic degradation, damage from microorganisms, damage from preparative mechanical shearing, damage from preparative enzymatic fragmentation, damage having naturally occurred in vivo, damage having occurred during nucleic acid extraction, damage having occurred during sequencing library preparation, damage having been introduced by a polymerase, damage having been introduced during nucleic acid repair, damage having occurred during nucleic acid end-tailing, damage having occurred during nucleic acid ligation, damage having occurred during sequencing, damage having occurred from mechanical handling of DNA, damage having occurred during passage through a nanopore, damage having occurred as part of aging in an organism, damage having occurred as a result if chemical exposure of an individual, damage having occurred by a mutagen, damage having occurred by a carcinogen, damage having occurred by a clastogen, damage having occurred from in vivo inflammation damage due to oxygen exposure, damage due to one or more strand breaks, and any combination thereof.

### II. Selected Embodiments of Duplex Sequencing Methods and Associated Adapters and Reagents

Duplex Sequencing is a method for producing error-corrected DNA sequences from double stranded nucleic acid molecules, and which was originally described in International Patent Publication No. WO 2013/142389 and in U.S. Pat. No. 9,752,188, and WO 2017/100441, in Schmitt et. al., PNAS, 2012 [1]; in Kennedy et. al., PLOS Genetics, 2013 [2]; in Kennedy et. al., Nature Protocols, 2014 [3]; and in Schmitt et. al., Nature Methods, 2015 [4]. Each of the above-mentioned patents, patent applications and publications are incorporated herein by reference in their entireties. As illustrated in FIGS. 1A-1C, and in certain aspects of the technology, Duplex Sequencing can be used to independently sequence both strands of individual DNA molecules in such a way that the derivative sequence reads can be recognized as having originated from the same double-stranded nucleic acid parent molecule during massively parallel sequencing (MPS), also commonly known as next generation sequencing (NGS), but also differentiated from each other as distinguishable entities following sequencing. The resulting sequence reads from each strand are then compared for the purpose of obtaining an error-corrected sequence of the original double-stranded nucleic acid molecule known as a Duplex Consensus Sequence (DCS). The process of Duplex Sequencing makes it possible to explicitly confirm that both strands of an original double stranded nucleic acid molecule are represented in the generated sequencing data used to form a DCS.

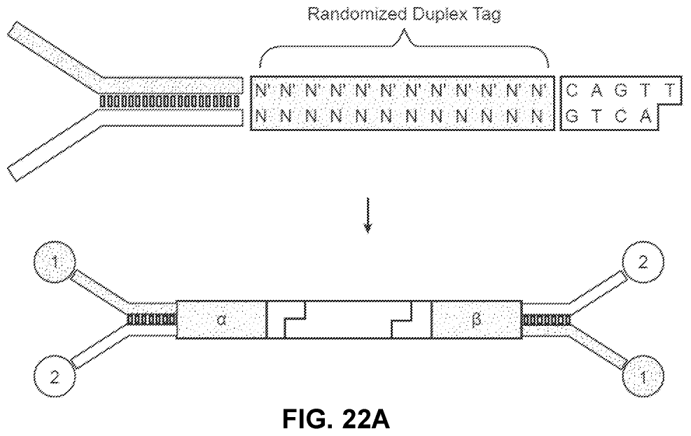

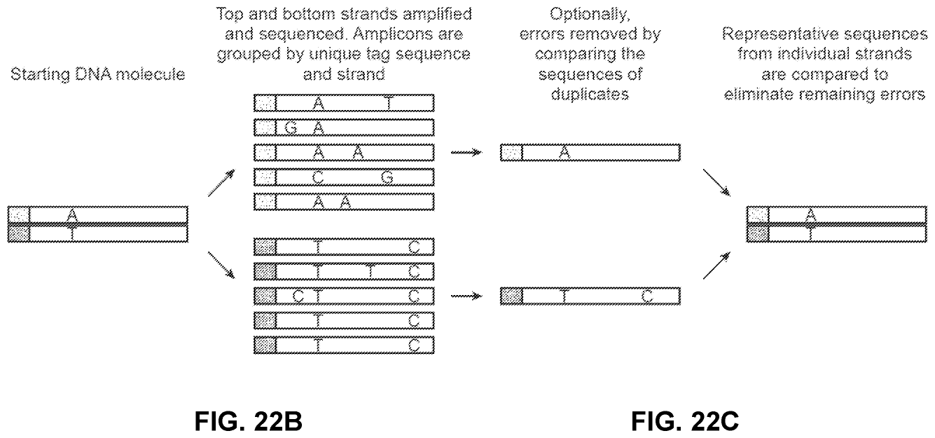

In certain embodiments, methods incorporating DS may include ligation of one or more sequencing adapters to a target double-stranded nucleic acid molecule, comprising a first strand target nucleic acid sequence and a second strand target nucleic sequence, to produce a double-stranded target nucleic acid complex (e.g. FIG. 22A).

In various embodiments, a resulting target nucleic acid complex can include at least one SMI sequence, which may entail an exogenously applied degenerate or semi-degenerate sequence (e.g., randomized duplex tag shown in FIG. 22A, sequences identified as α and β in FIG. 22A), endogenous information related to the specific shear-points of the target double-stranded nucleic acid molecule, or a combination thereof. The SMI can render the target-nucleic acid molecule substantially distinguishable from the plurality of other molecules in a population being sequenced either alone or in combination with distinguishing elements of the nucleic acid fragments to which they were ligated. The SMI element's substantially distinguishable feature can be independently carried by each of the single strands that form the double-stranded nucleic acid molecule such that the derivative amplification products of each strand can be recognized as having come from the same original substantially unique double-stranded nucleic acid molecule after sequencing. In other embodiments the SMI may include additional information and/or may be used in other methods for which such molecule distinguishing functionality is useful, such as those described in the above-referenced publications. In another embodiment, the SMI element may be incorporated after adapter ligation. In some embodiments the SMI is double-stranded in nature. In other embodiments it is single-stranded in nature (e.g., the SMI can be on the single-stranded portion(s) of the adapters). In other embodiments it is a combination of single-stranded and double-stranded in nature.

In some embodiments, each double-stranded target nucleic acid sequence complex can further include an element (e.g., an SDE) that renders the amplification products of the two single-stranded nucleic acids that form the target double-stranded nucleic acid molecule substantially distinguishable from each other after sequencing. In one embodiment, an SDE may comprise asymmetric primer sites comprised within the sequencing adapters, or, in other arrangements, sequence asymmetries may be introduced into the adapter molecules not within the primer sequences, such that at least one position in the nucleotide sequences of the first strand target nucleic acid sequence complex and the second stand of the target nucleic acid sequence complex are different from each other following amplification and sequencing. In other embodiments, the SMI may comprise another biochemical asymmetry between the two strands that differs from the canonical nucleotide sequences A, T, C, G or U, but is converted into at least one canonical nucleotide sequence difference in the two amplified and sequenced molecules. In yet another embodiment, the SDE may be a means of physically separating the two strands before amplification, such that the derivative amplification products from the first strand target nucleic acid sequence and the second strand target nucleic acid sequence are maintained in substantial physical isolation from one another for the purposes of maintaining a distinction between the two. Other such arrangements or methodologies for providing an SDE function that allows for distinguishing the first and second strands may be utilized, such as those described in the above-referenced publications, or other methods that serves the functional purpose described.

After generating the double-stranded target nucleic acid complex comprising at least one SMI and at least one SDE, or where one or both of these elements will be subsequently introduced, the complex can be subjected to DNA amplification, such as with PCR, or any other biochemical method of DNA amplification (e.g., rolling circle amplification, multiple displacement amplification, isothermal amplification, bridge amplification or surface-bound amplification, such that one or more copies of the first strand target nucleic acid sequence and one or more copies of the second strand target nucleic acid sequence are produced (e.g., FIG. 22B). The one or more amplification copies of the first strand target nucleic acid molecule and the one or more amplification copies of the second target nucleic acid molecule can then be subjected to DNA sequencing, preferably using a “Next-Generation” massively parallel DNA sequencing platform (e.g., FIG. 22B).

The sequence reads produced from either the first strand target nucleic acid molecule and the second strand target nucleic acid molecule derived from the original double-stranded target nucleic acid molecule can be identified based on sharing a related substantially unique SMI and distinguished from the opposite strand target nucleic acid molecule by virtue of an SDE. In some embodiments the SMI may be a sequence based on a mathematically-based error correction code (for example, a Hamming code), whereby certain amplification errors, sequencing errors or SMI synthesis errors can be tolerated for the purpose of relating the sequences of the SMI sequences on complementary strands of an original Duplex (e.g., a double-stranded nucleic acid molecule). For example, with a double stranded exogenous SMI where the SMI comprises 15 base pairs of fully degenerate sequence of canonical DNA bases, an estimated 4\hat{ }15=1,073,741,824 SMI variants will exist in a population of the fully degenerate SMIs. If two SMIs are recovered from reads of sequencing data that differ by only one nucleotide within the SMI sequence out of a population of 10,000 sampled SMIs, it can be mathematically calculated the probability of this occurring by random chance and a decision made whether it is more probable that the single base pair difference reflects one of the aforementioned types of errors and the SMI sequences could be determined to have in fact derived from the same original duplex molecule. In some embodiments where the SMI is, at least in part, an exogenously applied sequence where the sequence variants are not fully degenerate to each other and are, at least in part, known sequences, the identity of the known sequences can in some embodiments be designed in such a way that one or more errors of the aforementioned types will not convert the identity of one known SMI sequence to that of another SMI sequence, such that the probability of one SMI being misinterpreted as that of another SMI is reduced. In some embodiments this SMI design strategy comprises a Hamming Code approach or derivative thereof. Once identified, one or more sequence reads produced from the first strand target nucleic acid molecule are compared with one or more sequence reads produced from the second strand target nucleic acid molecule to produce an error-corrected target nucleic acid molecule sequence (e.g., FIG. 22C). For example, nucleotide positions where the bases from both the first and second strand target nucleic acid sequences agree are deemed to be true sequences, whereas nucleotide positions that disagree between the two strands are recognized as potential sites of technical errors that may be discounted, eliminated, corrected or otherwise identified. An error-corrected sequence of the original double-stranded target nucleic acid molecule can thus be produced (shown in FIG. 22C). In some embodiments and following separately grouping of each of the sequencing reads produced from the first strand target nucleic acid molecule and the second strand target nucleic acid molecule, a single-strand consensus sequence can be generated for each of the first and second strands. The single-stranded consensus sequences from the first strand target nucleic acid molecule and the second strand target nucleic acid molecule can then be compared to produce an error-corrected target nucleic acid molecule sequence (e.g., FIG. 22C).

Alternatively, in some embodiments, sites of sequence disagreement between the two strands can be recognized as potential sites of biologically-derived mismatches in the original double stranded target nucleic acid molecule. Alternatively, in some embodiments, sites of sequence disagreement between the two strands can be recognized as potential sites of DNA synthesis-derived mismatches in the original double stranded target nucleic acid molecule. Alternatively, in some embodiments, sites of sequence disagreement between the two strands can be recognized as potential sites where a damaged or modified nucleotide base was present on one or both strands and was converted to a mismatch by an enzymatic process (for example a DNA polymerase, a DNA glycosylase or another nucleic acid modifying enzyme or chemical process). In some embodiments, this latter finding can be used to infer the presence of nucleic acid damage or nucleotide modification prior to the enzymatic process or chemical treatment.

In some embodiments, and in accordance with aspects of the present technology, sequencing reads generated from the Duplex Sequencing steps discussed herein can be further filtered to eliminate sequencing reads from DNA-damaged molecules (e.g., damaged during storage, shipping, during or following tissue or blood extraction, during or following library preparation, etc.). For example, DNA repair enzymes, such as Uracil-DNA Glycosylase (UDG), Formamidopyrimidine DNA glycosylase (FPG), and 8-oxoguanine DNA glycosylase (OGG1), can be utilized to eliminate or correct DNA damage (e.g., in vitro DNA damage or in vivo damage). These DNA repair enzymes, for example, are glycoslyases that remove damaged bases from DNA. For example, UDG removes uracil that results from cytosine deamination (caused by spontaneous hydrolysis of cytosine) and FPG removes 8-oxo-guanine (e.g., a common DNA lesion that results from reactive oxygen species). FPG also has lyase activity that can generate a 1 base gap at abasic sites. Such abasic sites will generally subsequently fail to amplify by PCR, for example, because the polymerase fails to copy the template. Accordingly, the use of such DNA damage repair/elimination enzymes can effectively remove damaged DNA that doesn't have a true mutation but might otherwise be undetected as an error following sequencing and duplex sequence analysis. Although an error due to a damaged base can often be corrected by Duplex Sequencing in rare cases a complementary error could theoretically occur at the same position on both strands, thus, reducing error-increasing damage can reduce the probability of artifacts. Furthermore, during library preparation certain fragments of DNA to be sequenced may be single-stranded from their source or from processing steps (for example, mechanical DNA shearing). These regions are typically converted to double stranded DNA during an “end repair” step known in the art, whereby a DNA polymerase and nucleoside substrates are added to a DNA sample to extend 5′ recessed ends. A mutagenic site of DNA damage in the single-stranded portion of the DNA being copied (i.e. single-stranded 5′ overhang at one or both ends of the DNA duplex or internal single-stranded nicks or gaps) can cause an error during the fill-in reaction that could render a single-stranded mutation, synthesis error or site of nucleic acid damage into a double-stranded form that could be misinterpreted in the final duplex consensus sequence as a true mutation whereby the true mutation was present in the original double stranded nucleic acid molecule, when, in fact, it was not. This scenario, termed “pseudo-duplex”, can be reduced or prevented by use of such damage destroying/repair enzymes. In other embodiments this occurrence can be reduced or eliminated through use of strategies to destroy or prevent single-stranded portions of the original duplex molecule to form (e.g. use of certain enzymes being used to fragment the original double stranded nucleic acid material rather than mechanical shearing or certain other enzymes that may leave nicks or gaps). In other embodiments use of processes to eliminate single-stranded portions of original double-stranded nucleic acids (e.g. single-stand specific nucleases such as S1 nuclease or mung bean nuclease) can be utilized for a similar purpose.

In further embodiments, sequencing reads generated from the Duplex Sequencing steps discussed herein can be further filtered to eliminate false mutations by trimming ends of the reads most prone to pseudoduplex artifacts. For example, DNA fragmentation can generate single strand portions at the terminal ends of double-stranded molecule. These single-stranded portions can be filled in (e.g., by Klenow or T4 polymerase) during end repair. In some instances, polymerases make copy mistakes in these end repaired regions leading to the generation of “pseudoduplex molecules.” These artifacts of library preparation can incorrectly appear to be true mutations once sequenced. These errors, as a result of end repair mechanisms, can be eliminated or reduced from analysis post-sequencing by trimming the ends of the sequencing reads to exclude any mutations that may have occurred in higher risk regions, thereby reducing the number of false mutations. In one embodiment, such trimming of sequencing reads can be accomplished automatically (e.g., a normal process step). In another embodiment, a mutant frequency can be assessed for fragment end regions and if a threshold level of mutations is observed in the fragment end regions, sequencing read trimming can be performed before generating a double-strand consensus sequence read of the DNA fragments.

By way of specific example, in some embodiments, provided herein are methods of generating an error-corrected sequence read of a double-stranded target nucleic acid material, including the step of ligating a double-stranded target nucleic acid material to at least one adapter sequence, to form an adapter-target nucleic acid material complex, wherein the at least one adapter sequence comprises (a) a degenerate or semi-degenerate single molecule identifier (SMI) sequence that uniquely labels each molecule of the double-stranded target nucleic acid material, and (b) a first nucleotide adapter sequence that tags a first strand of the adapter-target nucleic acid material complex, and a second nucleotide adapter sequence that is at least partially non-complimentary to the first nucleotide sequence that tags a second strand of the adapter-target nucleic acid material complex such that each strand of the adapter-target nucleic acid material complex has a distinctly identifiable nucleotide sequence relative to its complementary strand. The method can next include the steps of amplifying each strand of the adapter-target nucleic acid material complex to produce a plurality of first strand adapter-target nucleic acid complex amplicons and a plurality of second strand adapter-target nucleic acid complex amplicons. The method can further include the steps of amplifying both the first and strands to provide a first nucleic acid product and a second nucleic acid product. The method may also include the steps of sequencing each of the first nucleic acid product and second nucleic acid product to produce a plurality of first strand sequence reads and plurality of second strand sequence reads, and confirming the presence of at least one first strand sequence read and at least one second strand sequence read. The method may further include comparing the at least one first strand sequence read with the at least one second strand sequence read, and generating an error-corrected sequence read of the double-stranded target nucleic acid material by discounting nucleotide positions that do not agree, or alternatively removing compared first and second strand sequence reads having one or more nucleotide positions where the compared first and second strand sequence reads are non-complementary.

By way of an additional specific example, in some embodiments, provided herein are methods of identifying a DNA variant from a sample including the steps of ligating both strands of a nucleic acid material (e.g., a double-stranded target DNA molecule) to at least one asymmetric adapter molecule to form an adapter-target nucleic acid material complex having a first nucleotide sequence associated with a first strand of a double-stranded target DNA molecule (e.g., a top strand) and a second nucleotide sequence that is at least partially non-complementary to the first nucleotide sequence associated with a second strand of the double-stranded target DNA molecule (e.g., a bottom strand), and amplifying each strand of the adapter-target nucleic acid material, resulting in each strand generating a distinct yet related set of amplified adapter-target nucleic acid products. The method can further include the steps of sequencing each of a plurality of first strand adapter-target nucleic acid products and a plurality of second strand adapter-target nucleic acid products, confirming the presence of at least one amplified sequence read from each strand of the adapter-target nucleic acid material complex, and comparing the at least one amplified sequence read obtained from the first strand with the at least one amplified sequence read obtained from the second strand to form a consensus sequence read of the nucleic acid material (e.g., a double-stranded target DNA molecule) having only nucleotide bases at which the sequence of both strands of the nucleic acid material (e.g., a double-stranded target DNA molecule) are in agreement, such that a variant occurring at a particular position in the consensus sequence read (e.g., as compared to a reference sequence) is identified as a true DNA variant.

In some embodiments, provided herein are methods of generating a high accuracy consensus sequence from a double-stranded nucleic acid material, including the steps of tagging individual duplex DNA molecules with an adapter molecule to form tagged DNA material, wherein each adapter molecule comprises (a) a degenerate or semi-degenerate single molecule identifier (SMI) that uniquely labels the duplex DNA molecule, and (b) first and second non-complementary nucleotide adapter sequences that distinguishes an original top strand from an original bottom strand of each individual DNA molecule within the tagged DNA material, for each tagged DNA molecule, and generating a set of duplicates of the original top strand of the tagged DNA molecule and a set of duplicates of the original bottom strand of the tagged DNA molecule to form amplified DNA material. The method can further include the steps of creating a first single strand consensus sequence (SSCS) from the duplicates of the original top strand and a second single strand consensus sequence (SSCS) from the duplicates of the original bottom strand, comparing the first SSCS of the original top strand to the second SSCS of the original bottom strand, and generating a high-accuracy consensus sequence having only nucleotide bases at which the sequence of both the first SSCS of the original top strand and the second SSCS of the original bottom strand are complimentary.

In further embodiments, provided herein are methods of detecting and/or quantifying DNA damage from a sample comprising double-stranded target DNA molecules including the steps of ligating both strands of each double-stranded target DNA molecule to at least one asymmetric adapter molecule to form a plurality of adapter-target DNA complexes, wherein each adapter-target DNA complex has a first nucleotide sequence associated with a first strand of a double-stranded target DNA molecule and a second nucleotide sequence that is at least partially non-complementary to the first nucleotide sequence associated with a second strand of the double-stranded target DNA molecule, and for each adapter target DNA complex: amplifying each strand of the adapter-target DNA complex, resulting in each strand generating a distinct yet related set of amplified adapter-target DNA amplicons. The method can further include the steps of sequencing each of a plurality of first strand adapter-target DNA amplicons and a plurality of second strand adapter-target DNA amplicons, confirming the presence of at least one sequence read from each strand of the adapter-target DNA complex, and comparing the at least one sequence read obtained from the first strand with the at least one sequence read obtained from the second strand to detect and/or quantify nucleotide bases at which the sequence read of one strand of the double-stranded DNA molecule is in disagreement (e.g., non-complimentary) with the sequence read of the other strand of the double-stranded DNA molecule, such that site(s) of DNA damage can be detected and/or quantified. In some embodiments, the method can further include the steps of creating a first single strand consensus sequence (SSCS) from the first strand adapter-target DNA amplicons and a second single strand consensus sequence (SSCS) from the second strand adapter-target DNA amplicons, comparing the first SSCS of the original first strand to the second SSCS of the original second strand, and identifying nucleotide bases at which the sequence of the first SSCS and the second SSCS are non-complementary to detect and/or quantify DNA damage associated with the double-stranded target DNA molecules in the sample.

Single Molecule Identifier Sequences (SMIs)

In accordance with various embodiments, provided methods and compositions include one or more SMI sequences on each strand of a nucleic acid material. The SMI can be independently carried by each of the single strands that result from a double-stranded nucleic acid molecule such that the derivative amplification products of each strand can be recognized as having come from the same original substantially unique double-stranded nucleic acid molecule after sequencing. In some embodiments, the SMI may include additional information and/or may be used in other methods for which such molecule distinguishing functionality is useful, as will be recognized by one of skill in the art. In some embodiments, an SMI element may be incorporated before, substantially simultaneously, or after adapter sequence ligation to a nucleic acid material.

In some embodiments, an SMI sequence may include at least one degenerate or semi-degenerate nucleic acid. In other embodiments, an SMI sequence may be non-degenerate. In some embodiments, the SMI can be the sequence associated with or near a fragment end of the nucleic acid molecule (e.g., randomly or semi-randomly sheared ends of ligated nucleic acid material). In some embodiments, an exogenous sequence may be considered in conjunction with the sequence corresponding to randomly or semi-randomly sheared ends of ligated nucleic acid material (e.g., DNA) to obtain an SMI sequence capable of distinguishing, for example, single DNA molecules from one another. In some embodiments, a SMI sequence is a portion of an adapter sequence that is ligated to a double-strand nucleic acid molecule. In certain embodiments, the adapter sequence comprising a SMI sequence is double-stranded such that each strand of the double-stranded nucleic acid molecule includes an SMI following ligation to the adapter sequence. In another embodiment, the SMI sequence is single-stranded before or after ligation to a double-stranded nucleic acid molecule and a complimentary SMI sequence can be generated by extending the opposite strand with a DNA polymerase to yield a complementary double-stranded SMI sequence. In other embodiments, an SMI sequence is in a single-stranded portion of the adapter (e.g., an arm of an adapter having a Y-shape). In such embodiments, the SMI can facilitate grouping of families of sequence reads derived from an original strand of a double-stranded nucleic acid molecule, and in some instances can confer relationship between original first and second strands of a double-stranded nucleic acid molecule (e.g., all or part of the SMIs maybe relatable via look up table). In embodiments, where the first and second strands are labeled with different SMIs, the sequence reads from the two original strands may be related using one or more of an endogenous SMI (e.g., a fragment-specific feature such as sequence associated with or near a fragment end of the nucleic acid molecule), or with use of an additional molecular tag shared by the two original strands (e.g., a barcode in a double-stranded portion of the adapter, or a combination thereof. In some embodiments, each SMI sequence may include between about 1 to about 30 nucleic acids (e.g., 1, 2, 3, 4, 5, 8, 10, 12, 14, 16, 18, 20, or more degenerate or semi-degenerate nucleic acids).

In some embodiments, a SMI is capable of being ligated to one or both of a nucleic acid material and an adapter sequence. In some embodiments, a SMI may be ligated to at least one of a T-overhang, an A-overhang, a CG-overhang, an overhang comprising a “sticky end” or single-stranded overhang region with known nucleotide length (e.g., 1, 2, 3, 4, 5, 6, 7, 8, 9, 10, 11, 12, 13, 14, 15, 16, 17, 18, 19, 20 or more nucleotides), a dehydroxylated base, and a blunt end of a nucleic acid material.

In some embodiments, a sequence of a SMI may be considered in conjunction with (or designed in accordance with) the sequence corresponding to, for example, randomly or semi-randomly sheared ends of a nucleic acid material (e.g., a ligated nucleic acid material), to obtain a SMI sequence capable of distinguishing single nucleic acid molecules from one another.

In some embodiments, at least one SMI may be an endogenous SMI (e.g., an SMI related to a shear point (e.g., a fragment end), for example, using the shear point itself or using a defined number of nucleotides in the nucleic acid material immediately adjacent to the shear point [e.g., 2, 3, 4, 5, 6, 7, 8, 9, 10 nucleotides from the shear point]). In some embodiments, at least one SMI may be an exogenous SMI (e.g., an SMI comprising a sequence that is not found on a target nucleic acid material).

In some embodiments, a SMI may be or comprise an imaging moiety (e.g., a fluorescent or otherwise optically detectable moiety). In some embodiments, such SMIs allow for detection and/or quantitation without the need for an amplification step.

In some embodiments a SMI element may comprise two or more distinct SMI elements that are located at different locations on the adapter-target nucleic acid complex.

Various embodiments of SMIs are further disclosed in International Patent Publication No. WO2017/100441, which is incorporated by reference herein in its entirety.

Strand-Defining Element (SDE)

In some embodiments, each strand of a double-stranded nucleic acid material may further include an element that renders the amplification products of the two single-stranded nucleic acids that form the target double-stranded nucleic acid material substantially distinguishable from each other after sequencing. In some embodiments, a SDE may be or comprise asymmetric primer sites comprised within a sequencing adapter, or, in other arrangements, sequence asymmetries may be introduced into the adapter sequences and not within the primer sequences, such that at least one position in the nucleotide sequences of a first strand target nucleic acid sequence complex and a second stand of the target nucleic acid sequence complex are different from each other following amplification and sequencing. In other embodiments, the SDE may comprise another biochemical asymmetry between the two strands that differs from the canonical nucleotide sequences A, T, C, G or U, but is converted into at least one canonical nucleotide sequence difference in the two amplified and sequenced molecules. In yet another embodiment, the SDE may be or comprise a means of physically separating the two strands before amplification, such that derivative amplification products from the first strand target nucleic acid sequence and the second strand target nucleic acid sequence are maintained in substantial physical isolation from one another for the purposes of maintaining a distinction between the two derivative amplification products. Other such arrangements or methodologies for providing an SDE function that allows for distinguishing the first and second strands may be utilized.

In some embodiments, a SDE may be capable of forming a loop (e.g., a hairpin loop). In some embodiments, a loop may comprise at least one endonuclease recognition site. In some embodiments the target nucleic acid complex may contain an endonuclease recognition site that facilitates a cleavage event within the loop. In some embodiments a loop may comprise a non-canonical nucleotide sequence. In some embodiments the contained non-canonical nucleotide may be recognizable by one or more enzyme that facilitates strand cleavage. In some embodiments the contained non-canonical nucleotide may be targeted by one or more chemical process facilitates strand cleavage in the loop. In some embodiments the loop may contain a modified nucleic acid linker that may be targeted by one or more enzymatic, chemical or physical process that facilitates strand cleavage in the loop. In some embodiments this modified linker is a photocleavable linker.

A variety of other molecular tools could serve as SMIs and SDEs. Other than shear points and DNA-based tags, single-molecule compartmentalization methods that keep paired strands in physical proximity or other non-nucleic acid tagging methods could serve the strand-relating function. Similarly, asymmetric chemical labelling of the adapter strands in a way that they can be physically separated can serve an SDE role. A recently described variation of Duplex Sequencing uses bisulfite conversion to transform naturally occurring strand asymmetries in the form of cytosine methylation into sequence differences that distinguish the two strands. Although this implementation limits the types of mutations that can be detected, the concept of capitalizing on native asymmetry is noteworthy in the context of emerging sequencing technologies that can directly detect modified nucleotides. Various embodiments of SDEs are further disclosed in International Patent Publication No. WO2017/100441, which is incorporated by reference in its entirety.

Adapters and Adapter Sequences

In various arrangements, adapter molecules that comprise SMIs (e.g., molecular barcodes), SDEs, primer sites, flow cell sequences and/or other features are contemplated for use with many of the embodiments disclosed herein. In some embodiments, provided adapters may be or comprise one or more sequences complimentary or at least partially complimentary to PCR primers (e.g., primer sites) that have at least one of the following properties: 1) high target specificity; 2) capable of being multiplexed; and 3) exhibit robust and minimally biased amplification.

In some embodiments, adapter molecules can be “Y”-shaped, “U”-shaped, “hairpin” shaped, have a bubble (e.g., a portion of sequence that is non-complimentary), or other features. In other embodiments, adapter molecules can comprise a “Y”-shape, a “U”-shaped, a “hairpin” shaped, or a bubble. Certain adapters may comprise modified or non-standard nucleotides, restriction sites, or other features for manipulation of structure or function in vitro. Adapter molecules may ligate to a variety of nucleic acid material having a terminal end. For example, adapter molecules can be suited to ligate to a T-overhang, an A-overhang, a CG-overhang, a multiple nucleotide overhang (also referred to herein as a “sticky end” or “sticky overhang”), a dehydroxylated base, a blunt end of a nucleic acid material and the end of a molecule were the 5′ of the target is dephosphorylated or otherwise blocked from traditional ligation. In other embodiments the adapter molecule can contain a dephosphorylated or otherwise ligation-preventing modification on the 5′ strand at the ligation site. In the latter two embodiments such strategies may be useful for preventing dimerization of library fragments or adapter molecules.

In some embodiments, adapter molecules can comprise a capture moiety suitable for isolating a desired target nucleic acid molecule ligated thereto.

An adapter sequence can mean a single-strand sequence, a double-strand sequence, a complimentary sequence, a non-complimentary sequence, a partial complimentary sequence, an asymmetric sequence, a primer binding sequence, a flow-cell sequence, a ligation sequence or other sequence provided by an adapter molecule. In particular embodiments, an adapter sequence can mean a sequence used for amplification by way of compliment to an oligonucleotide.

In some embodiments, provided methods and compositions include at least one adapter sequence (e.g., two adapter sequences, one on each of the 5′ and 3′ ends of a nucleic acid material). In some embodiments, provided methods and compositions may comprise 2 or more adapter sequences (e.g., 3, 4, 5, 6, 7, 8, 9, 10 or more). In some embodiments, at least two of the adapter sequences differ from one another (e.g., by sequence). In some embodiments, each adapter sequence differs from each other adapter sequence (e.g., by sequence). In some embodiments, at least one adapter sequence is at least partially non-complementary to at least a portion of at least one other adapter sequence (e.g., is non-complementary by at least one nucleotide).

In some embodiments, an adapter sequence comprises at least one non-standard nucleotide. In some embodiments, a non-standard nucleotide is selected from an abasic site, a uracil, tetrahydrofuran, 8-oxo-7,8-dihydro-2′deoxyadenosine (8-oxo-A), 8-oxo-7,8-dihydro-2′-deoxyguanosine (8-oxo-G), deoxyinosine, 5′nitroindole, 5-Hydroxymethyl-2′-deoxycytidine, iso-cytosine, 5′-methyl-isocytosine, or isoguanosine, a methylated nucleotide, an RNA nucleotide, a ribose nucleotide, an 8-oxo-guanine, a photocleavable linker, a biotinylated nucleotide, a desthiobiotin nucleotide, a thiol modified nucleotide, an acrydite modified nucleotide an iso-dC, an iso dG, a 2′-O-methyl nucleotide, an inosine nucleotide Locked Nucleic Acid, a peptide nucleic acid, a 5 methyl dC, a 5-bromo deoxyuridine, a 2,6-Diaminopurine, 2-Aminopurine nucleotide, an abasic nucleotide, a 5-Nitroindole nucleotide, an adenylated nucleotide, an azide nucleotide, a digoxigenin nucleotide, an I-linker, an 5′ Hexynyl modified nucleotide, an 5-Octadiynyl dU, photocleavable spacer, a non-photocleavable spacer, a click chemistry compatible modified nucleotide, and any combination thereof.

In some embodiments, an adapter sequence comprises a moiety having a magnetic property (i.e., a magnetic moiety). In some embodiments this magnetic property is paramagnetic. In some embodiments where an adapter sequence comprises a magnetic moiety (e.g., a nucleic acid material ligated to an adapter sequence comprising a magnetic moiety), when a magnetic field is applied, an adapter sequence comprising a magnetic moiety is substantially separated from adapter sequences that do not comprise a magnetic moiety (e.g., a nucleic acid material ligated to an adapter sequence that does not comprise a magnetic moiety).

In some embodiments, at least one adapter sequence is located 5′ to a SMI. In some embodiments, at least one adapter sequence is located 3′ to a SMI.

In some embodiments, an adapter sequence may be linked to at least one of a SMI and a nucleic acid material via one or more linker domains In some embodiments, a linker domain may be comprised of nucleotides. In some embodiments, a linker domain may include at least one modified nucleotide or non-nucleotide molecules (for example, as described elsewhere in this disclosure). In some embodiments, a linker domain may be or comprise a loop.

In some embodiments, an adapter sequence on either or both ends of each strand of a double-stranded nucleic acid material may further include one or more elements that provide a SDE. In some embodiments, a SDE may be or comprise asymmetric primer sites comprised within the adapter sequences.

In some embodiments, an adapter sequence may be or comprise at least one SDE and at least one ligation domain (i.e., a domain amendable to the activity of at least one ligase, for example, a domain suitable to ligating to a nucleic acid material through the activity of a ligase). In some embodiments, from 5′ to 3′, an adapter sequence may be or comprise a primer binding site, a SDE, and a ligation domain

Various methods for synthesizing Duplex Sequencing adapters have been previously described in, e.g., U.S. Pat. No. 9,752,188, International Patent Publication No. WO2017/100441, and International Patent Application No. PCT/US18/59908 (filed Nov. 8, 2018), all of which are incorporated by reference herein in their entireties.

Primers

In some embodiments, one or more PCR primers that have at least one of the following properties: 1) high target specificity; 2) capable of being multiplexed; and 3) exhibit robust and minimally biased amplification are contemplated for use in various embodiments in accordance with aspects of the present technology. A number of prior studies and commercial products have designed primer mixtures satisfying certain of these criteria for conventional PCR-CE. However, it has been noted that these primer mixtures are not always optimal for use with MPS. Indeed, developing highly multiplexed primer mixtures can be a challenging and time-consuming process. Conveniently, both Illumina and Promega have recently developed multiplex compatible primer mixtures for the Illumina platform that show robust and efficient amplification of a variety of standard and non-standard STR and SNP loci. Because these kits use PCR to amplify their target regions prior to sequencing, the 5′-end of each read in paired-end sequencing data corresponds to the 5′-end of the PCR primers used to amplify the DNA. In some embodiments, provided methods and compositions include primers designed to ensure uniform amplification, which may entail varying reaction concentrations, melting temperatures, and minimizing secondary structure and intra/inter-primer interactions. Many techniques have been described for highly multiplexed primer optimization for MPS applications. In particular, these techniques are often known as ampliseq methods, as well described in the art.

Amplification

Provided methods and compositions, in various embodiments, make use of, or are of use in, at least one amplification step wherein a nucleic acid material (or portion thereof, for example, a specific target region or locus) is amplified to form an amplified nucleic acid material (e.g., some number of amplicon products).

In some embodiments, amplifying a nucleic acid material includes a step of amplifying nucleic acid material derived from each of a first and second nucleic acid strand from an original double-stranded nucleic acid material using at least one single-stranded oligonucleotide at least partially complementary to a sequence present in a first adapter sequence such that a SMI sequence is at least partially maintained An amplification step further includes employing a second single-stranded oligonucleotide to amplify each strand of interest, and such second single-stranded oligonucleotide can be (a) at least partially complementary to a target sequence of interest, or (b) at least partially complementary to a sequence present in a second adapter sequence such that the at least one single-stranded oligonucleotide and a second single-stranded oligonucleotide are oriented in a manner to effectively amplify the nucleic acid material.

In some embodiments, amplifying nucleic acid material in a sample can include amplifying nucleic acid material in “tubes” (e.g., PCR tubes), in emulsion droplets, microchambers, and other examples described above or other known vessels. In some embodiments, amplifying nucleic acid material may comprise amplifying nucleic acid material in two or more (e.g., 3, 4, 5, 6, 7, 8, 9, 10, 20, 30, 40, 50 or more samples) physically separated samples (e.g., tubes, droplets, chambers, vessels, etc.). For example, an initial sample may be separated into multiple vessels prior to an amplification step. In some embodiments, each sample includes substantially the same amount of amplified nucleic acid material as each other sample, in some embodiments, at least two samples include substantially different amounts of amplified nucleic acid material.

In some embodiments, at least one amplifying step includes at least one primer that is or comprises at least one non-standard nucleotide. In some embodiments, a non-standard nucleotide is selected from a uracil, a methylated nucleotide, an RNA nucleotide, a ribose nucleotide, an 8-oxo-guanine, a biotinylated nucleotide, a locked nucleic acid, a peptide nucleic acid, a high-Tm nucleic acid variant, an allele discriminating nucleic acid variant, any other nucleotide or linker variant described elsewhere herein and any combination thereof.

While any application-appropriate amplification reaction is contemplated as compatible with some embodiments, by way of specific example, in some embodiments, an amplification step may be or comprise a polymerase chain reaction (PCR), rolling circle amplification (RCA), multiple displacement amplification (MDA), isothermal amplification, polony amplification within an emulsion, bridge amplification on a surface, the surface of a bead or within a hydrogel, and any combination thereof.

In some embodiments, amplifying a nucleic acid material includes use of single-stranded oligonucleotides at least partially complementary to regions of the adapter sequences on the 5′ and 3′ ends of each strand of the nucleic acid material. In some embodiments, amplifying a nucleic acid material includes use of at least one single-stranded oligonucleotide at least partially complementary to a target region or a target sequence of interest (e.g., a genomic sequence, a mitochondrial sequence, a plasmid sequence, a synthetically produced target nucleic acid, etc.) and a single-stranded oligonucleotide at least partially complementary to a region of the adapter sequence (e.g., a primer site).

In general, robust amplification, for example PCR amplification, can be highly dependent on the reaction conditions. Multiplex PCR, for example, can be sensitive to buffer composition, monovalent or divalent cation concentration, detergent concentration, crowding agent (i.e. PEG, glycerol, etc.) concentration, primer concentrations, primer Tms, primer designs, primer GC content, primer modified nucleotide properties, and cycling conditions (i.e. temperature and extension times and rate of temperature changes). Optimization of buffer conditions can be a difficult and time-consuming process. In some embodiments, an amplification reaction may use at least one of a buffer, primer pool concentration, and PCR conditions in accordance with a previously known amplification protocol. In some embodiments, a new amplification protocol may be created, and/or an amplification reaction optimization may be used. By way of specific example, in some embodiments, a PCR optimization kit may be used, such as a PCR Optimization Kit from Promega®, which contains a number of pre-formulated buffers that are partially optimized for a variety of PCR applications, such as multiplex, real-time, GC-rich, and inhibitor-resistant amplifications. These pre-formulated buffers can be rapidly supplemented with different Mg²⁺ and primer concentrations, as well as primer pool ratios. In addition, in some embodiments, a variety of cycling conditions (e.g., thermal cycling) may be assessed and/or used. In assessing whether or not a particular embodiment is appropriate for a particular desired application, one or more of specificity, allele coverage ratio for heterozygous loci, interlocus balance, and depth, among other aspects may be assessed. Measurements of amplification success may include DNA sequencing of the products, evaluation of products by gel or capillary electrophoresis or HPLC or other size separation methods followed by fragment visualization, melt curve analysis using double-stranded nucleic acid binding dyes or fluorescent probes, mass spectrometry or other methods known in the art.

In accordance with various embodiments, any of a variety of factors may influence the length of a particular amplification step (e.g., the number of cycles in a PCR reaction, etc.). For example, in some embodiments, a provided nucleic acid material may be compromised or otherwise suboptimal (e.g. degraded and/or contaminated). In such case, a longer amplification step may be helpful in ensuring a desired product is amplified to an acceptable degree. In some embodiments an amplification step may provide an average of 3 to 10 sequenced PCR copies from each starting DNA molecule, though in other embodiments, only a single copy of each of a first strand and second strand are required. Without wishing to be held to a particular theory, it is possible that too many or too few PCR copies could result in reduced assay efficiency and, ultimately, reduced depth. Generally, the number of nucleic acid (e.g., DNA) fragments used in an amplification (e.g., PCR) reaction is a primary adjustable variable that can dictate the number of reads that share the same SMI/barcode sequence.

Nucleic Acid Material

Types

In accordance with various embodiments, any of a variety of nucleic acid material may be used. In some embodiments, nucleic acid material may comprise at least one modification to a polynucleotide within the canonical sugar-phosphate backbone. In some embodiments, nucleic acid material may comprise at least one modification within any base in the nucleic acid material. For example, by way of non-limiting example, in some embodiments, the nucleic acid material is or comprises at least one of double-stranded DNA, single-stranded DNA, double-stranded RNA, single-stranded RNA, peptide nucleic acids (PNAs), locked nucleic acids (LNAs).

Sources

It is contemplated that nucleic acid material may come from any of a variety of sources. For example, in some embodiments, nucleic acid material is provided from a sample from at least one subject (e.g., a human or animal subject) or other biological source. In some embodiments, a nucleic acid material is provided from a banked/stored sample. In some embodiments, a sample is or comprises at least one of blood, serum, sweat, saliva, cerebrospinal fluid, mucus, uterine lavage fluid, a vaginal swab, a nasal swab, an oral swab, a tissue scraping, hair, a finger print, urine, stool, vitreous humor, peritoneal wash, sputum, bronchial lavage, oral lavage, pleural lavage, gastric lavage, gastric juice, bile, pancreatic duct lavage, bile duct lavage, common bile duct lavage, gall bladder fluid, synovial fluid, an infected wound, a non-infected wound, an archeological sample, a forensic sample, a water sample, a tissue sample, a food sample, a bioreactor sample, a plant sample, a fingernail scraping, semen, prostatic fluid, fallopian tube lavage, a cell free nucleic acid, a nucleic acid within a cell, a metagenomics sample, a lavage of an implanted foreign body, a nasal lavage, intestinal fluid, epithelial brushing, epithelial lavage, tissue biopsy, an autopsy sample, a necropsy sample, an organ sample, a human identification ample, an artificially produced nucleic acid sample, a synthetic gene sample, a nucleic acid data storage sample, tumor tissue, and any combination thereof. In other embodiments, a sample is or comprises at least one of a microorganism, a plant-based organism, or any collected environmental sample (e.g., water, soil, archaeological, etc.).

Modifications

In accordance with various embodiments, nucleic acid material may receive one or more modifications prior to, substantially simultaneously, or subsequent to, any particular step, depending upon the application for which a particular provided method or composition is used.

In some embodiments, a modification may be or comprise repair of at least a portion of the nucleic acid material. While any application-appropriate manner of nucleic acid repair is contemplated as compatible with some embodiments, certain exemplary methods and compositions therefore are described below and in the Examples.

By way of non-limiting example, in some embodiments, DNA repair enzymes, such as Uracil-DNA Glycosylase (UDG), Formamidopyrimidine DNA glycosylase (FPG), and 8-oxoguanine DNA glycosylase (OGG1), can be utilized to correct DNA damage (e.g., in vitro DNA damage). As discussed above, these DNA repair enzymes, for example, are glycoslyases that remove damaged bases from DNA. For example, UDG removes uracil that results from cytosine deamination (caused by spontaneous hydrolysis of cytosine) and FPG removes 8-oxo-guanine (e.g., most common DNA lesion that results from reactive oxygen species). FPG also has lyase activity that can generate 1 base gap at abasic sites. Such abasic sites will subsequently fail to amplify by PCR, for example, because the polymerase fails copy the template. Accordingly, the use of such DNA damage repair enzymes can effectively remove damaged DNA that doesn't have a true mutation, but might otherwise be undetected as an error following sequencing and duplex sequence analysis.

As discussed above, in further embodiments, sequencing reads generated from the processing steps discussed herein can be further filtered to eliminate false mutations by trimming ends of the reads most prone to artifacts. For example, DNA fragmentation can generate single-strand portions at the terminal ends of double-stranded molecules. These single-stranded portions can be filled in (e.g., by Klenow) during end repair. In some instances, polymerases make copy mistakes in these end-repaired regions leading to the generation of “pseudoduplex molecules.” These artifacts can appear to be true mutations once sequenced. These errors, as a result of end repair mechanisms, can be eliminated from analysis post-sequencing by trimming the ends of the sequencing reads to exclude any mutations that may have occurred, thereby reducing the number of false mutations. In some embodiments, such trimming of sequencing reads can be accomplished automatically (e.g., a normal process step). In some embodiments, a mutant frequency can be assessed for fragment end regions and if a threshold level of mutations is observed in the fragment end regions, sequencing read trimming can be performed before generating a double-strand consensus sequence read of the DNA fragments.

Some embodiments of DS methods provide PCR-based targeted enrichment strategies compatible with the use of molecular barcodes for error correction. For example, sequencing enrichment strategy utilizing Separated PCRs of Linked Templates for sequencing (“SPLiT-DS”) method steps may also benefit from pre-enriched nucleic acid material using one or more of the embodiments described herein. SPLiT-DS was originally described in International Patent Publication No. WO/20181175997, which is incorporated herein by reference in its entirety. A SPLiT-DS approach can begin with labelling (e.g., tagging) fragmented double-stranded nucleic acid material (e.g., from a DNA sample) with molecular barcodes in a similar manner as described above and with respect to a standard DS library construction protocol. In some embodiments, the double-stranded nucleic acid material may be fragmented (e.g., such as with cell free DNA, damaged DNA, etc.); however, in other embodiments, various steps can include fragmentation of the nucleic acid material using mechanical shearing such as sonication, or other DNA cutting methods, such as described further herein. Aspects of labelling the fragmented double-stranded nucleic acid material can include end-repair and 3′-dA-tailing, if required in a particular application, followed by ligation of the double-stranded nucleic acid fragments with DS adapters containing an SMI. In other embodiments, the SMI can be endogenous or a combination of exogenous and endogenous sequence for uniquely relating information from both strands of an original nucleic acid molecule. Following ligation of adapter molecules to the double-stranded nucleic acid material, the method can continue with amplification (e.g., PCR amplification, rolling circle amplification, multiple displacement amplification, isothermal amplification, bridge amplification, surface-bound amplification, etc.).

In certain embodiments, primers specific to, for example, one or more adapter sequences, can be used to amplify each strand of the nucleic acid material resulting in multiple copies of nucleic acid amplicons derived from each strand of an original double strand nucleic acid molecule, with each amplicon retaining the originally associated SMI. After amplification and associated steps to remove reaction byproducts, the sample can be split (preferably, but not necessarily, substantially evenly) into two or more separate samples (e.g., in tubes, in emulsion droplets, in microchambers, isolated droplets on a surface, or other known vessels, collectively referred to as “tube(s)”). Following separation, and in accordance with one embodiment of SPLiT-DS process, the method can include amplifying the first strand in a first sample through use of a primer specific to a first adapter sequence to provide a first nucleic acid product, and amplifying the second strand in a second sample through use of a primer specific to a second adapter sequence to provide a second nucleic acid product. Next, the method can include sequencing each of the first nucleic acid product and second nucleic acid product, and comparing the sequence of the first nucleic acid product to the sequence of the second nucleic acid product. In some embodiments, a nucleic acid material comprises an adapter sequence on each of the 5′ and 3′ ends of each strand of the nucleic acid material. In certain applications, amplification of the individual strands in separated samples can be accomplished using a single-stranded oligonucleotide at least partially complementary to a target sequence of interest such that the single molecule identifier sequence is at least partially maintained

### SELECTED EXAMPLES OF APPLICATIONS

As is described herein, provided methods and compositions may be used for any of a variety of purposes and/or in any of a variety of scenarios. Below are described examples of non-limiting applications and/or scenarios for the purposes of specific illustration only.

Monitoring Response to Therapies (Tumor Mutation, etc.)

The advent of next-generation sequencing (NGS) in genomic research has enabled the characterization of the mutational landscape of tumors with unprecedented detail and has resulted in the cataloguing of diagnostic, prognostic, and clinically actionable mutations. Collectively, these mutations hold significant promise for improved cancer outcomes through personalized medicine as well as for potential early cancer detection and screening. Prior to the present disclosure, a critical limitation in the field has been the inability to detect these mutations when they are present at low frequency. Clinical biopsies are often comprised mostly of normal cells and the detection of cancer cells based on their DNA mutations is a technological challenge even for modern NGS. The identification of tumor mutations amongst thousands of normal genomes is analogous to finding a needle in a haystack, requiring a level of sequencing accuracy beyond previously known methods.

Generally, this problem is aggravated in the case of liquid biopsies, where the challenge is not only to provide the extreme sensitivity required to find tumor mutations, but also to do so with the minimal amounts of DNA typically present in these biopsies. The term ‘liquid biopsy’ typically refers to blood in its ability to inform about cancer based on the presence of circulating tumor DNA (ctDNA). ctDNA is shed by cancer cells into the bloodstream and has shown great promise to monitor, detect and predict cancer as well as to enable tumor genotyping and therapy selection. These applications could revolutionize the current management of patients with cancer, however, progress has been slower than previously anticipated. A major issue is that ctDNA typically represents a very small portion of all the cell-free DNA (cfDNA) present in plasma. In metastatic cancers its frequency could be >5%, but in localized cancers is only between 1%-0.001%. In theory, DNA subpopulations of any size should be detectable by assaying a sufficient number of molecules. However, a fundamental limitation of previous methods is the high frequency with which bases are scored incorrectly. Errors often arise during cluster generation, sequencing cycles, poor cluster resolution, and template degradation. The result is that approximately 0.1-1% of sequenced bases are called incorrectly. Further issues can arise from polymerase mistakes and amplification bias during PCR that can result in skewed populations or the introduction of false mutant allele frequencies (MAF). Taken together, previously known techniques, including conventional NGS, are incapable of performing at the level required for the detection of low frequency mutations.

Due to its high accuracy, DS as well as methods for increasing conversion and workflow efficiency of these sequencing platforms hold promise in the oncology field. As is described herein, provided methods and compositions allow for an innovative approach to the DS methodology that integrates the double strand molecular tagging of DS with target nucleic acid enrichment for increased efficiency and scalability while maintaining error correction.

In addition to the need for an assay that is highly accurate and efficient, the realities of the clinical laboratory also demand assays that are fast, scalable, and reasonably cost effective. Accordingly, various embodiments in accordance with aspects of the present technology that improve workflow efficiency of DS (e.g., enrichment strategy for DS) is highly desirable. Digestion/size selection enrichment and affinity-based enrichment of specific target sequences for DS applications, as described herein provide high target specificity, performance on low DNA inputs, scalability, and minimal cost.

Some embodiments of provided methods and compositions are especially significant for cancer research in general and for the field of ctDNA in particular, as the technology developed herein has the potential to identify cancer mutations with unprecedented sensitivity while minimizing DNA input, preparation time, and costs. Target nucleic acid enrichment embodiments disclosed herein can be useful for clinical applications that could significantly increase survival through improved patient management and early cancer detection.

Patient Stratification

Patient stratification, which generally refers to the partitioning of patients based on one or more non-treatment-related factors, is a topic of significant interest in the medical community. Much of this interest may be due to the fact that certain therapeutic candidates have failed to receive FDA approval, in part to a previously unrecognized difference among the patients in a trial. These differences may be or include one or more genetic differences that result in a therapeutic being metabolized differently, or in side effects being present or exacerbated in one group of patients vs one or more other groups of patients. In some cases, some or all of these differences may be detected as one or more distinct genetic profile(s) in the patient(s) that result in a reaction to the therapeutic that is different from other patients that do not exhibit the same genetic profile.

Accordingly, in some embodiments, provided methods and compositions may be useful in determining which subject(s) in a particular patient population (e.g., patients suffering from a common disease, disorder or condition) may respond to a particular therapy. For example, in some embodiments, provided methods and/or compositions may be used to assess whether or not a particular subject possesses a genotype that is associated with poor response to the therapy. In some embodiments, provided methods and/or compositions may be used to assess whether or not a particular subject possesses a genotype that is associated with positive response to the therapy.

Forensics

Previous approaches to forensic DNA analysis relied almost entirely on capillary electrophoretic separation of PCR amplicons to identify length polymorphisms in short tandem repeat sequences. This type of analysis has proven to be extremely valuable since its introduction in 1991. Since that time, several publications have introduced standardized protocols, validated their use in laboratories worldwide, detailed its use on many different population groups, and introduced more efficient approaches, such as miniSTRs.

While this approach has proven to be extremely successful, the technology has a number of drawbacks that limit its utility. For example, current approaches to STR genotyping often give rise to background signal resulting from PCR stutter, caused by slippage of the polymerase on the template DNA. This issue is especially important in samples with more than one contributor, due to the difficulty in distinguishing the stutter alleles from genuine alleles. Another issue arises when analyzing degraded DNA samples. Variation in fragment length often results in significantly lower, or even absent, longer PCR fragments. As a consequence, profiles from degraded DNA often have lower power of discrimination

The introduction of MPS systems has the potential to address several challenging issues in forensics analysis. For example, these platforms offer unparalleled capacity to allow for the simultaneous analysis of STRs and SNPs in nuclear and mtDNA, which will dramatically increase the power of discrimination between individuals and offers the possibility to determine ethnicity and even physical attributes. Furthermore, unlike PCR-CE, which simply reports the average genotype of an aggregate population of molecules, MPS technology digitally tabulates the full nucleotide sequence of many individual DNA molecules, thus offering the unique ability to detect MAFs within a heterogeneous DNA mixture. Because forensics specimens comprising two or more contributors remains one of the most problematic issues in forensics, the impact of MPS on the field of forensics could be enormous.

The publication of the human genome highlighted the immense power of MPS platforms. However, until fairly recently, the full power of these platforms was of limited use to forensics due to the read lengths being significantly shorter than the STR loci, precluding the ability to call length-based genotypes. Initially, pyrosequencers, such as the Roche 454 platform, were the only platforms with sufficient read length to sequence the core STR loci. However, read lengths in competing technologies have increased, thus bringing their utility for forensics applications into play. A number of studies have revealed the potential for MPS genotyping of STR loci. Overall, the general outcome of all these studies, regardless of the platform, is that STRs can be successfully typed producing genotypes comparable with CE analyses, even from compromised forensic samples.

While all of these studies show concordance with traditional PCR-CE approaches, and even indicate additional benefits like the detection of intra-STR SNPs, they have also highlighted a number of current issues with the technology. For example, current MPS approaches to STR genotyping rely on multiplex PCR to both provide enough DNA to sequence and introduce PCR primers. However, because multiplex PCR kits were designed for PCR-CE, they contain primers for various sized amplicons. This variation results in coverage imbalance with a bias toward amplification of smaller fragments, which can result in allele drop-out. Indeed, recent studies have shown that differences in PCR efficiency can affect mixture components, especially at low MAFs. To address this issue, several sequencing kits specifically designed for forensics are now commercially available and validation studies are beginning to be reported. However, due to the high level of multiplexing, amplification biases are still evident.

Like PCR-CE, MPS is not immune to the occurrence of PCR stutter. The vast majority of MPS studies on STR report the occurrence of artifactual drop-in alleles. Recently, systematic MPS studies report that most stutter events appear as shorter length polymorphisms that differ from the true allele in four base-pair units, with the most common being n−4, but with n−8 and n−12 positions also being observed. The percent stutter typically occurred in ˜1% of reads, but can be as high as 3% at some loci, indicating that MPS can exhibit stutter at higher rates than PCR-CE.

In contrast, in some embodiments, provided methods and compositions allow for high quality and efficient sequencing of low quality and/or low amount samples, as described above and in the Examples below. Accordingly, in some embodiments, provided methods and/or compositions may be useful for rare variant detection of the DNA from one individual intermixed at low abundance with the DNA of another individual of a different genotype.

Forensic DNA samples commonly contain non-human DNA. Potential sources of this extraneous DNA are: the source of the DNA (e.g., microbes in saliva or buccal samples), the surface environment from which the sample was collected, and contamination from the laboratory (e.g. reagents, work area, etc.). Another aspect provided by some embodiments is that certain provided methods and compositions allow for the distinguishing of contaminating nucleic acid material from other sources (e.g., different species) and/or surface or environmental contaminants so that these materials (and/or their effects) may be removed from the final analysis and not bias the sequencing results.

In highly degraded DNA, the loci specific PCR may not work well due to the DNA fragments not containing the requisite primer annealing site, resulting in allelic dropout. This situation would limit the uniqueness of genotype calls and the confidence of matches is less assured, especially in the mixture trials. However, in some embodiments, provided methods and compositions allow for the use of single nucleotide polymorphisms (SNPs) in addition to or as an alternative to STR markers.

In fact, with ever increasing data on human genetic variation, SNPs are increasingly relevant for forensic work. As such, in some embodiments, provided methods and compositions use a primer design strategy such that multiplex primer panels may be created, for example, based on currently available sequencing kits, which virtually ensure reads traverse one or more SNP locations.

### FURTHER EXAMPLES

1. A method for enriching target nucleic acid material, comprising:
providing a nucleic acid material;
cutting the nucleic acid material with one or more targeted endonucleases so that a target region of predetermined length is separated from the rest of the nucleic acid material;
enzymatically destroying non-targeted nucleic acid material;
releasing the target region of predetermined length from the targeted endonuclease; and
analyzing the cut target region.

2. The method of example 1, wherein enzymatically destroying non-targeted nucleic acid material comprises providing an exonuclease enzyme.

3. The method of example 1, wherein enzymatically destroying non-targeted nucleic acid material comprises providing one or more of an exonuclease enzyme and an endonuclease enzyme.

4. The method of example 1, wherein the destroying comprises at least one of enzymatic digestion and enzymatic cleavage.

5. The method of any one of example 1-4, wherein the one or more targeted endonucleases remain bound to the target region during the enzymatically destroying step.

6. The method of any one of examples 1-5, wherein at least one targeted endonuclease is a ribonucleoprotein complex comprising a capture label, and wherein the target region of predetermined length is physically separated from the rest of the nucleic acid via the capture label while the at least one targeted endonuclease remains bound to the target region.

7. The method of example 1-5, wherein at least one targeted endonuclease is a ribonucleoprotein complex comprising a capture label, and wherein the method further comprises capturing the target region with an extraction moiety configured to bind the capture label.

8. The method of example 6 or example 7, wherein a capture label is or comprises at least one of Aciydite, azide, azide (NHS ester), digoxigenin (NHS ester), Thinker, Amino modifier C6, Amino modifier C12, Amino modifier C6 dT, Unilink amino modifier, hexynyl, 5-octadiynyl dU, biotin, biotin (azide), biotin dT, biotin TEG, dual biotin, PC biotin, desthiobiotin TEG, thiol modifier C3, dithiol, thiol modifier C6 S—S, succinyl groups.

9. The method of example 7, wherein an extraction moiety is or comprises at least one of amino silane, epoxy silane, isothiocyanate, aminophenyl silane, aminpropyl silane, mercapto silane, aldehyde, epoxide, phosphonate, streptavidin, avidin, a hapten recognizing an antibody, a particular nucleic acid sequence, magnetically attractable particles (Dynabeads), photolabile resins.

10. The method of example 7, wherein the extraction moiety is bound to a surface.

11. The method of example 7, wherein the target region is physically separated after enzymatically destroying the non-targeted nucleic acid material.

12. The method of any one of examples 1-11, wherein the one or more targeted endonucleases is selected from the group consisting of a ribonucleoprotein, a Cas enzyme, a Cas9-like enzyme, a Cpf1 enzyme, a meganuclease, a transcription activator-like effector-based nuclease (TALEN), a zinc-finger nuclease, an argonaute nuclease or a combination thereof.

13. The method of any one of examples 1-12, wherein the one or more targeted endonucleases comprises Cas9 or CPF1 or a derivative thereof.

14. The method of any one of examples 1-13, wherein cutting the nucleic acid material includes cutting the nucleic acid material with one or more targeted endonucleases such that more than one target nucleic acid fragments of substantially known length are formed.

15. The method of example 14, further comprising isolating the more than one target nucleic acid fragments based on the predetermined length.

16. The method of example 15, wherein the target nucleic acid fragments are of different substantially known lengths.

17. The method of example 15, wherein the target nucleic acid fragments each comprise a genomic sequence of interest from one or more different locations in a genome.

18. The method of example 15, wherein the target nucleic acid fragments each comprise a targeted sequence from a substantially known region within the nucleic acid material.

19. The method of any one of examples 15-18, wherein isolating the target nucleic acid fragment based on the substantially known length includes enriching for the target nucleic acid fragment by gel electrophoresis, gel purification, liquid chromatography, size exclusion purification, filtration or SPRI bead purification.

20. The method of example 1, further comprising ligating at least one SMI and/or adapter sequence to at least one of the 5′ or 3′ ends of the cut target region of predetermined length.

21. The method of example 1, wherein analyzing comprises quantitation and/or sequencing of the target region.

22. The method of example 21, wherein quantitation comprises at least one of spectrophotometric analysis, real-time PCR, and/or fluorescence-based quantitation.

23. The method of example 21, wherein sequencing comprises duplex sequencing, SPLiT-duplex sequencing, Sanger sequencing, shotgun sequencing, bridge amplification/sequencing, nanopore sequencing, single molecule real-time sequencing, ion torrent sequencing, pyrosequencing, digital sequencing (e.g., digital barcode-based sequencing), direct digital sequencing, sequencing by ligation, polony-based sequencing, electrical current-based sequencing (e.g., tunneling currents), sequencing via mass spectroscopy, microfluidics-based sequencing, and any combination thereof.

24. The method of example 21, wherein sequencing comprises:
sequencing a first strand of the target region to generate a first strand sequence read;
sequencing a second strand of the target region to generate a second strand sequence read; and
comparing the first strand sequence read to the second strand sequence read to generate an error-corrected sequence read.

25. The method of example 24, wherein the error-corrected sequence read comprises nucleotide bases that agree between the first strand sequence read and the second strand sequence read.

26. The method of example 24 or example 25, wherein a variation occurring at a particular position in the error-corrected sequence read is identified as a true variant.

27. The method of any one of examples 24-26, wherein a variation that occurs at a particular position in only one of the first strand sequence read or the second strand sequence read is identified as a potential artifact.

28. The method of any one of examples 24-27, wherein the error-corrected sequence read is used to identify or characterize a cancer, a cancer risk, a cancer mutation, a cancer metabolic state, a mutator phenotype, a carcinogen exposure, a toxin exposure, a chronic inflammation exposure, an age, a neurodegenerative disease, a pathogen, a drug resistant variant, a fetal molecule, a forensically relevant molecule, an immunologically relevant molecule, a mutated T-cell receptor, a mutated B-cell receptor, a mutated immunoglobulin locus, a kategis site in a genome, a hypermutable site in a genome, a low frequency variant, a subclonal variant, a minority population of molecules, a source of contamination, a nucleic acid synthesis error, an enzymatic modification error, a chemical modification error, a gene editing error, a gene therapy error, a piece of nucleic acid information storage, a microbial quasispecies, a viral quasispecies, an organ transplant, an organ transplant rejection, a cancer relapse, residual cancer after treatment, a preneoplastic state, a dysplastic state, a microchimerism state, a stem cell transplant state, a cellular therapy state, a nucleic acid label affixed to another molecule, or a combination thereof in an organism or subject from which the double-stranded target nucleic acid molecule is derived.

29. The method of any one of examples 24-27, wherein the error-corrected sequence read is used to identify a mutagenic compound or exposure.

30. The method of any one of examples 24-27, wherein the error-corrected sequence read is used to identify a carcinogenic compound or exposure.

31. The method of any one of example 24-27, wherein the nucleic acid material is derived from a forensics sample, and wherein the error-corrected sequence read is used in a forensic analysis.

32. The method of example 1, wherein the targeted endonuclease comprises at least one of a CRISPR-associated (Cas) enzyme, a ribonucleoprotein complex, a homing endonuclease, a zinc-fingered nuclease, a transcription activator-like effector nuclease (TALEN), an argonaute nuclease, and/or a megaTAL nuclease.

33. The method of example 32, wherein the CRISPR-associated (Cas) enzyme is Cas9 or Cpf1.

34. The method of example 32, wherein the CRISPR-associated (Cas) enzyme is Cpf1, and wherein the target region comprises a 5′ overhang and a 3′ overhang of predetermined or known nucleotide sequence.

35. The method of example 1, wherein cutting the nucleic acid material with a targeted endonuclease comprises cutting the nucleic acid material with more than one targeted endonuclease.

36. The method of example 35, wherein the more than one targeted endonuclease comprises more than one Cas enzyme directed to more than one target region.

37. The method of example 35, wherein cutting the nucleic acid material with a targeted endonuclease so that a target region of predetermined length is separated from the rest of the nucleic acid material comprises cutting the target region with a pair of targeted endonucleases directed to cut the nucleic acid material at a predetermined distance apart so as to generate the target region having the predetermined length.

38. The method of example 37, wherein the pair of target endonucleases comprise a pair of Cas enzymes.

39. The method of example 38, wherein the pair of Cas enzymes comprise the same type of Cas enzyme.

40. The method of example 38, wherein the pair of Cas enzymes comprise two different types of Cas enzymes.

41. A method for enriching target nucleic acid material, comprising:
providing a nucleic acid material;
cutting the nucleic acid material with one or more targeted endonucleases so that a target region of predetermined length is separated from the rest of the nucleic acid material, wherein at least one targeted endonuclease comprises a capture label;
capturing the target region of predetermined length with an extraction moiety configured to bind the capture label;
releasing the target region of predetermined length from the targeted endonuclease; and
analyzing the cut target region.

42. A method for enriching target nucleic acid material, comprising:
providing a nucleic acid material;
binding a catalytically inactive CRISPR-associated (Cas) enzymes to a target region of the nucleic acid material;
enzymatically treating the nucleic acid material with one or more nucleic acid digesting enzymes such that non-targeted nucleic acid material is destroyed and the target region is protected from the digesting enzymes by the bound catalytically inactive Cas enzyme;
releasing the target region from the catalytically inactive Cas enzyme; and
analyzing the target region.

43. The method of example 42, wherein the binding step comprises binding a pair of catalytically inactive Cas enzymes to the target region such that nucleic acid material between the bound Cas enzymes is enzymatically protected from the digesting enzymes, thereby enriching the target nucleic acid material for the target region.

44. The method of example 42, wherein the catalytically inactive Cas enzyme comprises a capture label and wherein the method further comprises capturing the target region with an extraction moiety configured to bind the capture label.

45. The method of example 42, further comprising enriching the target region by size selection.

46. A method for enriching target nucleic acid material, comprising:
providing a nucleic acid material;
providing a pair of catalytically active targeted endonucleases and at least one catalytically inactive targeted endonuclease comprising a capture label, wherein the catalytically inactive targeted endonuclease is directed to bind the target region of the nucleic acid material, and wherein the pair of catalytically active targeted endonucleases are directed to bind the target region on either side of the catalytically inactive targeted endonuclease;
cutting the nucleic acid material with the pair of catalytically active targeted endonucleases so that the target region is separated from the rest of the nucleic acid material;
capturing the target region with an extraction moiety configured to bind the capture label;
releasing the target region from the targeted endonucleases; and
analyzing the cut target region.

47. A method for enriching target nucleic acid material from a sample comprising a plurality of nucleic acid fragments, comprising:
providing one or more catalytically inactive CRISPR-associated (Cas) enzymes having a capture label to the sample comprising target nucleic acid fragments and non-target nucleic acid fragments, wherein the one or more catalytically inactive Cas enzymes are configured to bind the target nucleic acid fragments;
providing a surface comprising an extraction moiety configured to bind the capture label; and
separating the target nucleic acid fragments from the non-target nucleic acid fragments by capturing the target nucleic acid fragments via binding the capture label by the extraction moiety.

48. The method of example 47, further comprising attaching adapter molecules to ends of the plurality of nucleic acid fragments prior to providing the one or more catalytically inactive CRISPR-associated (Cas) enzymes.

49. A method for enriching target double-stranded nucleic acid material, comprising:
providing a nucleic acid material;
cutting the nucleic acid material with one or more targeted endonucleases to generate a double-stranded target nucleic acid fragment comprising 5′ sticky end having a 5′ predetermined nucleotide sequence and/or a 3′ sticky end having a 3′ predetermined nucleotide sequence; and
separating the double-stranded target nucleic acid molecule from the rest of the nucleic acid material via at least one of the 5′ sticky end and the 3′ sticky end.

50. The method of example 49, further comprising providing at least one sequencing adapter molecule comprising a ligatable end at least partially complementary to the 5′ predetermined nucleotide sequence or the 3′ predetermined nucleotide sequence;
ligating the at least one sequencing adapter molecule to the double-stranded target nucleic acid molecule; and
analyzing the double-stranded target nucleic acid fragment via sequencing.

51. The method of example 50 wherein the at least one adapter molecule comprises a Y-shape or a U-shape.

52. The method of example 50, wherein the at least one adapter molecule is a hairpin molecule.

53. The method of example 50, wherein the at least one adapter molecule comprises a capture molecule configured to be bound by an extraction moiety.

54. The method of example 50, wherein a sequencing adapter molecule is ligated to each of the 5′ sticky end and the 3′ sticky end of the double-stranded target nucleic acid fragment.

55. The method of example 49, wherein separating the double-stranded target nucleic acid molecule from the rest of the nucleic acid material via at least one of the 5′ sticky end and the 3′ sticky end comprises providing an oligonucleotide having a sequence at least partially complementary to the 5′ predetermined nucleotide sequence or the 3′ predetermined nucleotide sequence.

56. The method of example 55, wherein the oligonucleotide is bound to a surface.

57. The method of example 55, wherein the oligonucleotide comprises a capture label configured to bind an extraction moiety.

58. The method of example 49, wherein the one or more targeted endonucleases comprises Cpf1.

59. The method of example 49, wherein the one or more targeted endonucleases comprises a Cas9 nickase.

60. A kit for enriching target nucleic acid material, comprising:
nucleic acid library, comprising
   nucleic acid material; and
   a plurality of catalytically inactive Cas enzymes, wherein the Cas enzymes comprise a tag having a sequence code,
   wherein the plurality of Cas enzymes are bound to a plurality of site-specific target regions along the nucleic acid material;
a plurality of probes, wherein each probe comprises
   an oligonucleotide sequence comprising a complement to a corresponding sequence code; and a capture label; and
a look-up table cataloguing the relationship between the site-specific target regions, the sequence code associated with the site-specific target region, and the probe comprising the complement to a corresponding sequence code.

61. The method of any one of the above examples, wherein the nucleic acid material is or comprises at least one of double-stranded DNA and double-stranded RNA.

62. The method of any one of the above examples, wherein at least some of the nucleic acid material is damaged.

63. The method of example 62, wherein the damage is or comprises at least one of oxidation, alkylation, deamination, methylation, hydrolysis, hydroxylation, nicking, intra-strand crosslinks, inter-strand cross links, blunt end strand breakage, staggered end double strand breakage, phosphorylation, dephosphorylation, sumoylation, glycosylation, deglycosylation, putrescinylation, carboxylation, halogenation, formylation, single-stranded gaps, damage from heat, damage from desiccation, damage from UV exposure, damage from gamma radiation damage from X-radiation, damage from ionizing radiation, damage from non-ionizing radiation, damage from heavy particle radiation, damage from nuclear decay, damage from beta-radiation, damage from alpha radiation, damage from neutron radiation, damage from proton radiation, damage from cosmic radiation, damage from high pH, damage from low pH, damage from reactive oxidative species, damage from free radicals, damage from peroxide, damage from hypochlorite, damage from tissue fixation such formalin or formaldehyde, damage from reactive iron, damage from low ionic conditions, damage from high ionic conditions, damage from unbuffered conditions, damage from nucleases, damage from environmental exposure, damage from fire, damage from mechanical stress, damage from enzymatic degradation, damage from microorganisms, damage from preparative mechanical shearing, damage from preparative enzymatic fragmentation, damage having naturally occurred in vivo, damage having occurred during nucleic acid extraction, damage having occurred during sequencing library preparation, damage having been introduced by a polymerase, damage having been introduced during nucleic acid repair, damage having occurred during nucleic acid end-tailing, damage having occurred during nucleic acid ligation, damage having occurred during sequencing, damage having occurred from mechanical handling of DNA, damage having occurred during passage through a nanopore, damage having occurred as part of aging in an organism, damage having occurred as a result if chemical exposure of an individual, damage having occurred by a mutagen, damage having occurred by a carcinogen, damage having occurred by a clastogen, damage having occurred from in vivo inflammation damage due to oxygen exposure, damage due to one or more strand breaks, and any combination thereof.

64. The method of any one of the above examples, wherein the nucleic acid material is provided from a sample comprising one or more double stranded nucleic acid molecules originating from a subject or an organism.

65. The method of example 64, wherein the sample is or comprises a body tissue, a biopsy, a skin sample, blood, serum, plasma, sweat, saliva, cerebrospinal fluid, mucus, uterine lavage fluid, a vaginal swab, a pap smear, a nasal swab, an oral swab, a tissue scraping, hair, a finger print, urine, stool, vitreous humor, peritoneal wash, sputum, bronchial lavage, oral lavage, pleural lavage, gastric lavage, gastric juice, bile, pancreatic duct lavage, bile duct lavage, common bile duct lavage, gall bladder fluid, synovial fluid, an infected wound, a non-infected wound, an archaeological sample, a forensic sample, a water sample, a tissue sample, a food sample, a bioreactor sample, a plant sample, a bacterial sample, a protozoan sample, a fungal sample, an animal sample, a viral sample, a multi-organism sample, a fingernail scraping, semen, prostatic fluid, vaginal fluid, a vaginal swab, a fallopian tube lavage, a cell free nucleic acid, a nucleic acid within a cell, a metagenomics sample, a lavage or a swab of an implanted foreign body, a nasal lavage, intestinal fluid, epithelial brushing, epithelial lavage, tissue biopsy, an autopsy sample, a necropsy sample, an organ sample, a human identification sample, a non-human identification sample, an artificially produced nucleic acid sample, a synthetic gene sample, a banked or stored sample, tumor tissue, a fetal sample, an organ transplant sample, a microbial culture sample, a nuclear DNA sample, a mitochondrial DNA sample, a chloroplast DNA sample, an apicoplast DNA sample, an organelle sample, and any combination thereof.

66. The method of any one of the above examples, wherein the nucleic acid material comprises nucleic acid molecules of a substantially or near uniform length.

67 The method of any one of any one of the above examples, wherein the target nucleic acid material originates from a subject or an organism.

68. The method of any one of any one of the above examples, wherein the target nucleic acid material has been at least partially artificially synthesized.

69. The method of any one of the above examples, wherein at most 1000 ng of nucleic acid material is initially provided.

70. The method of any one of the above examples, wherein at most 10 ng of nucleic acid material is initially provided.

71. The method of any one of the above examples, wherein the nucleic acid material comprises nucleic acid material derived from more than one source.

### Equivalents and Scope

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments. All references cited herein are incorporated by reference as if fully set forth herein.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

Those skilled in the art will recognize, or be able to ascertain using no more than routine experimentation, many equivalents to the specific embodiments of the disclosed technology described herein. The scope of the present technology is not intended to be limited to the above Description, but rather is as set forth in the following claims:

1. A method for enriching target nucleic acid material, comprising:
providing a nucleic acid material;
cutting the nucleic acid material with one or more targeted endonucleases so that a target region of predetermined length is separated from the rest of the nucleic acid material;
enzymatically destroying non-targeted nucleic acid material;
releasing the target region of predetermined length from the targeted endonuclease; and
analyzing the cut target region.

2. The method of claim 1, wherein enzymatically destroying non-targeted nucleic acid material comprises providing an exonuclease enzyme.

3. The method of claim 1, wherein enzymatically destroying non-targeted nucleic acid material comprises providing one or more of an exonuclease enzyme and an endonuclease enzyme.

4. The method of claim 1, wherein the destroying comprises at least one of enzymatic digestion and enzymatic cleavage.

5. The method of any one of claim 1-4, wherein the one or more targeted endonucleases remain bound to the target region during the enzymatically destroying step.

6. The method of any one of claims 1-5, wherein at least one targeted endonuclease is a ribonucleoprotein complex comprising a capture label, and wherein the target region of predetermined length is physically separated from the rest of the nucleic acid via the capture label while the at least one targeted endonuclease remains bound to the target region.

7. The method of claim 1-5, wherein at least one targeted endonuclease is a ribonucleoprotein complex comprising a capture label, and wherein the method further comprises capturing the target region with an extraction moiety configured to bind the capture label.

8. The method of claim 6 or claim 7, wherein a capture label is or comprises at least one of Acrydite, azide, azide (NHS ester), digoxigenin (NHS ester), Winker, Amino modifier C6, Amino modifier C12, Amino modifier C6 dT, Unilink amino modifier, hexynyl, 5-octadiynyl dU, biotin, biotin (azide), biotin dT, biotin TEG, dual biotin, PC biotin, desthiobiotin TEG, thiol modifier C3, dithiol, thiol modifier C6 S—S, succinyl groups.

9. The method of claim 7, wherein an extraction moiety is or comprises at least one of amino silane, epoxy silane, isothiocyanate, aminophenyl silane, aminpropyl silane, mercapto silane, aldehyde, epoxide, phosphonate, streptavidin, avidin, a hapten recognizing an antibody, a particular nucleic acid sequence, magnetically attractable particles (Dynabeads), photolabile resins.

10. The method of claim 7, wherein the extraction moiety is bound to a surface.

11. The method of claim 7, wherein the target region is physically separated after enzymatically destroying the non-targeted nucleic acid material.

12. The method of any one of claims 1-11, wherein the one or more targeted endonucleases is selected from the group consisting of a ribonucleoprotein, a Cas enzyme, a Cas9-like enzyme, a Cpf1 enzyme, a meganuclease, a transcription activator-like effector-based nuclease (TALEN), a zinc-finger nuclease, an argonaute nuclease or a combination thereof.

13. The method of any one of claims 1-12, wherein the one or more targeted endonucleases comprises Cas9 or CPF1 or a derivative thereof.

14. The method of any one of claims 1-13, wherein cutting the nucleic acid material includes cutting the nucleic acid material with one or more targeted endonucleases such that more than one target nucleic acid fragments of substantially known length are formed.

15. The method of claim 14, further comprising isolating the more than one target nucleic acid fragments based on the predetermined length.

16. The method of claim 15, wherein the target nucleic acid fragments are of different substantially known lengths.

17. The method of claim 15, wherein the target nucleic acid fragments each comprise a genomic sequence of interest from one or more different locations in a genome.

18. The method of claim 15, wherein the target nucleic acid fragments each comprise a targeted sequence from a substantially known region within the nucleic acid material.

19. The method of any one of claims 15-18, wherein isolating the target nucleic acid fragment based on the substantially known length includes enriching for the target nucleic acid fragment by gel electrophoresis, gel purification, liquid chromatography, size exclusion purification, filtration or SPRI bead purification.

20. The method of claim 1, further comprising ligating at least one SMI and/or adapter sequence to at least one of the 5′ or 3′ ends of the cut target region of predetermined length.

21. The method of claim 1, wherein analyzing comprises quantitation and/or sequencing of the target region.

22. The method of claim 21, wherein quantitation comprises at least one of spectrophotometric analysis, real-time PCR, and/or fluorescence-based quantitation.

23. The method of claim 21, wherein sequencing comprises duplex sequencing, SPLiT-duplex sequencing, Sanger sequencing, shotgun sequencing, bridge amplification/sequencing, nanopore sequencing, single molecule real-time sequencing, ion torrent sequencing, pyrosequencing, digital sequencing (e.g., digital barcode-based sequencing), direct digital sequencing, sequencing by ligation, polony-based sequencing, electrical current-based sequencing (e.g., tunneling currents), sequencing via mass spectroscopy, microfluidics-based sequencing, and any combination thereof.

24. The method of claim 21, wherein sequencing comprises:
sequencing a first strand of the target region to generate a first strand sequence read;
sequencing a second strand of the target region to generate a second strand sequence read; and
comparing the first strand sequence read to the second strand sequence read to generate an error-corrected sequence read.

25. The method of claim 24, wherein the error-corrected sequence read comprises nucleotide bases that agree between the first strand sequence read and the second strand sequence read.

26. The method of claim 24 or claim 25, wherein a variation occurring at a particular position in the error-corrected sequence read is identified as a true variant.

27. The method of any one of claims 24-26, wherein a variation that occurs at a particular position in only one of the first strand sequence read or the second strand sequence read is identified as a potential artifact.

28. The method of any one of claims 24-27, wherein the error-corrected sequence read is used to identify or characterize a cancer, a cancer risk, a cancer mutation, a cancer metabolic state, a mutator phenotype, a carcinogen exposure, a toxin exposure, a chronic inflammation exposure, an age, a neurodegenerative disease, a pathogen, a drug resistant variant, a fetal molecule, a forensically relevant molecule, an immunologically relevant molecule, a mutated T-cell receptor, a mutated B-cell receptor, a mutated immunoglobulin locus, a kategis site in a genome, a hypermutable site in a genome, a low frequency variant, a subclonal variant, a minority population of molecules, a source of contamination, a nucleic acid synthesis error, an enzymatic modification error, a chemical modification error, a gene editing error, a gene therapy error, a piece of nucleic acid information storage, a microbial quasispecies, a viral quasispecies, an organ transplant, an organ transplant rejection, a cancer relapse, residual cancer after treatment, a preneoplastic state, a dysplastic state, a microchimerism state, a stem cell transplant state, a cellular therapy state, a nucleic acid label affixed to another molecule, or a combination thereof in an organism or subject from which the double-stranded target nucleic acid molecule is derived.

29. The method of any one of claims 24-27, wherein the error-corrected sequence read is used to identify a mutagenic compound or exposure.

30. The method of any one of claims 24-27, wherein the error-corrected sequence read is used to identify a carcinogenic compound or exposure.

31. The method of any one of claim 24-27, wherein the nucleic acid material is derived from a forensics sample, and wherein the error-corrected sequence read is used in a forensic analysis.

32. The method of claim 1, wherein the targeted endonuclease comprises at least one of a CRISPR-associated (Cas) enzyme, a ribonucleoprotein complex, a homing endonuclease, a zinc-fingered nuclease, a transcription activator-like effector nuclease (TALEN), an argonaute nuclease, and/or a megaTAL nuclease.

33. The method of claim 32, wherein the CRISPR-associated (Cas) enzyme is Cas9 or Cpf1.

34. The method of claim 32, wherein the CRISPR-associated (Cas) enzyme is Cpf1, and wherein the target region comprises a 5′ overhang and a 3′ overhang of predetermined or known nucleotide sequence.

35. The method of claim 1, wherein cutting the nucleic acid material with a targeted endonuclease comprises cutting the nucleic acid material with more than one targeted endonuclease.

36. The method of claim 35, wherein the more than one targeted endonuclease comprises more than one Cas enzyme directed to more than one target region.

37. The method of claim 35, wherein cutting the nucleic acid material with a targeted endonuclease so that a target region of predetermined length is separated from the rest of the nucleic acid material comprises cutting the target region with a pair of targeted endonucleases directed to cut the nucleic acid material at a predetermined distance apart so as to generate the target region having the predetermined length.

38. The method of claim 37, wherein the pair of target endonucleases comprise a pair of Cas enzymes.

39. The method of claim 38, wherein the pair of Cas enzymes comprise the same type of Cas enzyme.

40. The method of claim 38, wherein the pair of Cas enzymes comprise two different types of Cas enzymes.

41. A method for enriching target nucleic acid material, comprising:
providing a nucleic acid material;
cutting the nucleic acid material with one or more targeted endonucleases so that a target region of predetermined length is separated from the rest of the nucleic acid material, wherein at least one targeted endonuclease comprises a capture label;
capturing the target region of predetermined length with an extraction moiety configured to bind the capture label;
releasing the target region of predetermined length from the targeted endonuclease; and
analyzing the cut target region.

42. A method for enriching target nucleic acid material, comprising:
providing a nucleic acid material;
binding a catalytically inactive CRISPR-associated (Cas) enzymes to a target region of the nucleic acid material;
enzymatically treating the nucleic acid material with one or more nucleic acid digesting enzymes such that non-targeted nucleic acid material is destroyed and the target region is protected from the digesting enzymes by the bound catalytically inactive Cas enzyme;
releasing the target region from the catalytically inactive Cas enzyme; and
analyzing the target region.

43. The method of claim 42, wherein the binding step comprises binding a pair of catalytically inactive Cas enzymes to the target region such that nucleic acid material between the bound Cas enzymes is enzymatically protected from the digesting enzymes, thereby enriching the target nucleic acid material for the target region.

44. The method of claim 42, wherein the catalytically inactive Cas enzyme comprises a capture label and wherein the method further comprises capturing the target region with an extraction moiety configured to bind the capture label.

45. The method of claim 42, further comprising enriching the target region by size selection.

46. A method for enriching target nucleic acid material, comprising:
providing a nucleic acid material;
providing a pair of catalytically active targeted endonucleases and at least one catalytically inactive targeted endonuclease comprising a capture label, wherein the catalytically inactive targeted endonuclease is directed to bind the target region of the nucleic acid material, and wherein the pair of catalytically active targeted endonucleases are directed to bind the target region on either side of the catalytically inactive targeted endonuclease;
cutting the nucleic acid material with the pair of catalytically active targeted endonucleases so that the target region is separated from the rest of the nucleic acid material;
capturing the target region with an extraction moiety configured to bind the capture label;
releasing the target region from the targeted endonucleases; and
analyzing the cut target region.

47. A method for enriching target nucleic acid material from a sample comprising a plurality of nucleic acid fragments, comprising:
providing one or more catalytically inactive CRISPR-associated (Cas) enzymes having a capture label to the sample comprising target nucleic acid fragments and non-target nucleic acid fragments, wherein the one or more catalytically inactive Cas enzymes are configured to bind the target nucleic acid fragments;
providing a surface comprising an extraction moiety configured to bind the capture label; and
separating the target nucleic acid fragments from the non-target nucleic acid fragments by capturing the target nucleic acid fragments via binding the capture label by the extraction moiety.

48. The method of claim 47, further comprising attaching adapter molecules to ends of the plurality of nucleic acid fragments prior to providing the one or more catalytically inactive CRISPR-associated (Cas) enzymes.

49. A method for enriching target double-stranded nucleic acid material, comprising:
providing a nucleic acid material;
cutting the nucleic acid material with one or more targeted endonucleases to generate a double-stranded target nucleic acid fragment comprising 5′ sticky end having a 5′ predetermined nucleotide sequence and/or a 3′ sticky end having a 3′ predetermined nucleotide sequence; and
separating the double-stranded target nucleic acid molecule from the rest of the nucleic acid material via at least one of the 5′ sticky end and the 3′ sticky end.

50. The method of claim 49, further comprising providing at least one sequencing adapter molecule comprising a ligatable end at least partially complementary to the 5′ predetermined nucleotide sequence or the 3′ predetermined nucleotide sequence;
ligating the at least one sequencing adapter molecule to the double-stranded target nucleic acid molecule; and
analyzing the double-stranded target nucleic acid fragment via sequencing.

51. The method of claim 50 wherein the at least one adapter molecule comprises a Y-shape or a U-shape.

52. The method of claim 50, wherein the at least one adapter molecule is a hairpin molecule.

53. The method of claim 50, wherein the at least one adapter molecule comprises a capture molecule configured to be bound by an extraction moiety.

54. The method of claim 50, wherein a sequencing adapter molecule is ligated to each of the 5′ sticky end and the 3′ sticky end of the double-stranded target nucleic acid fragment.

55. The method of claim 49, wherein separating the double-stranded target nucleic acid molecule from the rest of the nucleic acid material via at least one of the 5′ sticky end and the 3′ sticky end comprises providing an oligonucleotide having a sequence at least partially complementary to the 5′ predetermined nucleotide sequence or the 3′ predetermined nucleotide sequence.

56. The method of claim 55, wherein the oligonucleotide is bound to a surface.

57. The method of claim 55, wherein the oligonucleotide comprises a capture label configured to bind an extraction moiety.

58. The method of claim 49, wherein the one or more targeted endonucleases comprises Cpf1.

59. The method of claim 49, wherein the one or more targeted endonucleases comprises a Cas9 nickase.

60. A kit for enriching target nucleic acid material, comprising:
nucleic acid library, comprising
nucleic acid material; and
a plurality of catalytically inactive Cas enzymes, wherein the Cas enzymes comprise a tag having a sequence code,
wherein the plurality of Cas enzymes are bound to a plurality of site-specific target regions along the nucleic acid material;
a plurality of probes, wherein each probe comprises
an oligonucleotide sequence comprising a complement to a corresponding sequence code; and
a capture label; and
a look-up table cataloguing the relationship between the site-specific target regions, the sequence code associated with the site-specific target region, and the probe comprising the complement to a corresponding sequence code.
